(19) United States
(12) Patent Application Publication
(10) Pub. No.: US 2024/0158340 A1
(43) Pub. Date: May 16, 2024

(54) PHOTOCATALYTIC ARYLATION OF CARBONYL COMPOUNDS AND METHODS FOR USING THE SAME

(71) Applicant: Arizona Board of Regents on Behalf of the University of Arizon, Tucson, AZ (US)

(72) Inventors: Thomas L. Gianetti, Tucson, AZ (US); Md Mubarak Hossain, Tucson, AZ (US)

(21) Appl. No.: 18/289,543

(22) PCT Filed: May 5, 2022
(86) PCT No.: PCT/US2022/027786

§ 371 Date: Nov. 3, 2023

Related U.S. Application Data
(60) Provisional application No. 63/185,910, filed on May 7, 2021.
(60) Provisional application No. 63/281,334, filed on Nov. 19, 2021.

(51) Int. Cl. C07C 253/30; B01J 31/02; C07C 45/68

(52) U.S. Cl. CPC C07C 253/30; B01J 31/0244; C07C 45/68

(57) ABSTRACT

The present invention relates to a method for producing an α-aryl substituted carbonyl compound (e.g., an α-aryl substituted cyclic ketone) from a carbonyl compound (e.g., a cyclic ketone) using an aryl halide or a heteroaryl halide and a photocatalyst (e.g., acridinium, helicenium, angulenium, or a combination thereof) in the presence of an amine compound. The method of the present invention is particularly useful in producing an α-aryl substituted carbonyl compound (e.g., an α-aryl substituted cyclic ketone) from an unactivated carbonyl compound (e.g., an unactivated cyclic ketone).

This application claims the benefit of U.S. Provisional Application No. 63/185,910, filed on May 7, 2021, and U.S. Provisional Application No. 63/281,334, filed on Nov. 19, 2021, the entire contents of each of which are hereby incorporated by reference.

### FIELD OF THE INVENTION

The present invention relates to a method for producing an α-aryl substituted carbonyl compound (e.g., an α-aryl substituted cyclic ketone) from a carbonyl compound (e.g., a cyclic ketone) using an aryl halide or a heteroaryl halide and a photocatalyst (e.g., acridinium, helicenium, angulenium, or a combination thereof) in the presence of an amine compound. The method of the present invention is particularly useful in producing an α-aryl substituted carbonyl compound (e.g., an α-aryl substituted cyclic ketone) from an unactivated carbonyl compound (e.g., an unactivated cyclic ketone).

### BACKGROUND OF THE INVENTION

Functionalization of C—H bonds has evolved as a fundamental principle that underpins both academic and industrial importance in conceptualization and actualization of challenging organic transformations. This ubiquitous but relatively inert bond, if converted to a more valuable C—C bond, can greatly accelerate the preparation of a wide variety of compounds. This transformation is particularly sought after in fine chemicals and pharmaceutical applications. Incorporation of an aryl substituent at the α-position of carbonyl compounds is a powerful and attractive method to construct a C(sp²)-C(sp³) bond; and its coupling products, especially α-aryl cyclic ketones are significant structural motifs that are often encountered in a wide range of biologically interesting natural and pharmaceutical products. See below.

### Selected Bioactive Compounds with α-aryl Cyclic Ketone Scaffold

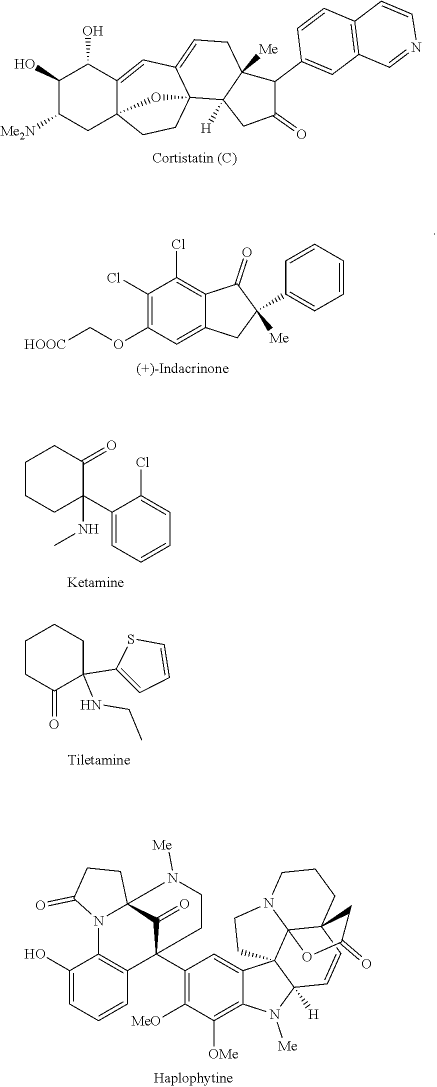

A number of classical stoichiometric transformations have been employed for such a key transformation, but suffered from complications in scale-up, either due to highly activated aryl halides or the use of stoichiometric amount of toxic reagents with harsh reaction conditions, e.g., a high reaction temperature (FIG. 1B). Significant efforts have been directed towards the development of alternative synthetic routes to this important scaffold. With advances in metal-catalyzed transformations, palladium-catalyzed α-arylations of the carbonyl compounds appeared to be the most promising route due to its versatility. However, the high cost of the catalyst and additives, as well as requiring a high reaction temperature, limits the utility of this approach in the synthesis of α-aryl carbonyls as building block, especially for pharmaceutical applications.

There is therefore a continuing need for synthetic methods for producing α-aryl carbonyl compounds that use inexpensive reagents, are industry viable, and do not require high reaction temperatures.

### SUMMARY OF THE INVENTION

The present inventors have surprisingly found that photocatalysts (e.g., non-metal based photocatalysts) can be used to produce α-arylated carbonyl compounds.

In one aspect, the present invention relates to a method for producing an α-aryl substituted carbonyl compound (e.g., an α-aryl substituted cyclic ketone). In one embodiment, the method comprises irradiating a solution mixture comprising:
an unactivated carbonyl compound (e.g., an unactivated cyclic ketone compound);
an optionally substituted aryl halide or an optionally substituted heteroaryl halide;
an amine compound (e.g., a secondary amine compound); and
a photocatalyst;
with visible light under conditions sufficient to produce the α-aryl substituted carbonyl compound.

Another aspect of the present invention provides a method for producing an α-aryl substituted cyclic ketone. In one embodiment, the method includes irradiating a solution mixture comprising:
a cyclic ketone compound;
an aryl halide;
a secondary amine compound; and
a photocatalyst comprising an acridinium, helicenium, angulenium, or a combination thereof;
with light under conditions sufficient to produce an α-aryl substituted cyclic ketone.

In some embodiments, the cyclic ketone compound is an unactivated cyclic ketone compound. Still in other embodiments, the cyclic ketone compound comprises a heterocycloalkyl ketone or a cycloalkyl ketone. Yet in other embodiments, the cyclic ketone is a substituted cyclic ketone. In one particular embodiment, the cyclic ketone is a 5-, 6-, or 7-membered cyclic ketone.

In further embodiments, the heterocycloalkyl ketone comprises a heterocyclic moiety selected from the group consisting of tetrahydro-2H-thiopyran, piperidine, tetrahydro-2H-pyran, tetrahydrothiophene, pyrrolidine, tetrahydrofuran, azepane, thiepane, and oxepane.

In some embodiments, the method according to any embodiment described herein is conducted at a temperature of about 50° C. or less, such as about 40° C.

In yet other embodiments of the methods described herein, the photocatalyst comprises acridinium, helicenium, angulenium, or a combination thereof. See, e.g., International Publication No. WO 21/155331, which is incorporated herein by reference in its entirety.

Other suitable photocatalysts include, but are not limited to, acridinium based photocatalysts such as hexamethoxy acridiniums, a 9-mesityl-acridinium compound; heliceniums; anguleniums; xanthene based photocatalysts such as rhodamine-6G, eosin Y; carbazolyl based photocatalysts such as 1,2,3,5-tetrakis(carbazol-9-yl)-4,6-dicyanobenzene, 2,4,5,6-tetrakis(9H-carbazol-9-yl) isophthalonitrile (4-CzIPN); quinoliniums; benzophenones; quinones; pyryliums; cyanoarenes; perylene diimides; a Ru based photocatalyst such as Ru(bpy)3 compound and known derivatives; an Ir based photocatalyst such as Ir(ppy)3, [Ir(dtbbpy)(ppy)2]PF6 and known derivatives; or any combination of any of the foregoing.

In some embodiments of any of the methods described herein, the optionally substituted aryl halide or optionally substituted heteroaryl halide is of the formula:

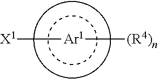

wherein
n is 0 to x, where x is the maximum number of substituents possible on Ar¹;
X¹ is Cl, Br or I;
Ar¹ is aryl or heteroaryl; and each R⁴ is, independently, alkyl, halide, haloalkyl, a carbonyl group, cyano, —CO₂R^{a}, —OR^{b}, —NR^{c}R^{d}, an amide, —SR^{a}, —BR^{e}R^{f}, or —PR^{e}R^{f}, wherein each R^{a} is independently, alkyl, R^{b} is alkyl or a hydroxy protecting group, R^{c} is H or alkyl, and R^{d} is H, alkyl or an amine protecting group, R^{e} and R^{f} are each independently H, alkyl, hydroxy or an alkoxy group, or R^{e} and R^{f} together with the atom to which they are attached form an optionally substituted cyclic moiety.

In some embodiments, n is 0. In some embodiments, n is 1. In some embodiments, n is 2. In some embodiments, n is 3. In some embodiments, n is 4. In some embodiments, n is 5. In some embodiments, n is 0, 1, 2, 3, 4 or 5.

Yet in other embodiments, Ar¹ is phenyl, naphthyl, anthracyl, pyridyl, furanyl, thiophenyl, thiazolyl, isothiazolyl, triazolyl, triazinyl, imidazolyl, oxazolyl, isoxazolyl, pyrrolyl, pyrazolyl, pyrazinyl, pyrimidinyl, benzofuranyl, isobenzofuranyl, benzothiazolyl, benzoisothiazolyl, benzotriazolyl, indolyl, isoindolyl, benzoxazolyl, thiazolyl, isothiazolyl, quinolyl, isoquinolyl, benzimidazolyl, benzisoxazolyl, benzothiophenyl, dibenzofuran, or benzodiazepin-2-one-5-yl, each of which may be optionally substituted.

Yet in other embodiments of any of the methods disclosed herein, the aryl halide has a reduction potential less than or equal to excited state oxidation potential of the photocatalyst. Still in other embodiments of any of the methods disclosed herein, said aryl halide comprise at least one substituent. In other embodiments, the aryl halide comprises an aryl ring moiety selected from the group consisting of phenyl, antracenyl, and naphthyl. Still yet in other embodiments of any of the methods disclosed herein, said heteroaryl halide comprises a heteroaryl ring moiety selected from the group consisting of pyridyl, furanyl, thiophenyl, thiazolyl, isothiazolyl, triazolyl, triazinyl, imidazolyl, oxazolyl, isoxazolyl, pyrrolyl, pyrazolyl, pyrazinyl, pyrimidinyl, benzofuranyl, isobenzofuranyl, benzothiazolyl, benzoisothiazolyl, benzotriazolyl, indolyl, isoindolyl, benzoxazolyl, thiazolyl, isothiazolyl, quinolyl, isoquinolyl, benzimidazolyl, benzisoxazolyl, benzothiophenyl, dibenzofuran, and benzodiazepin-2-one-5-yl.

In additional embodiments of any of the methods described herein, the amine compound (e.g., secondary amine compound) is a cyclic amine compound (e.g., cyclic secondary amine compound). In additional embodiments of any of the methods described herein, the amine compound (e.g., secondary amine compound) is a 5, 6 or 7-membered cyclic amine compound. In other embodiments of any of the methods described herein, the amine compound (e.g., secondary amine compound) comprises azepane, piperidine, pyrrolidine, or any combination thereof.

Another aspect of the present invention relates to a method for producing an α-aryl substituted compound of formula (I):

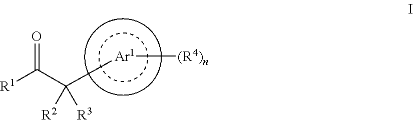

In one embodiment, the method comprises irradiating an electromagnetic radiation to a solution mixture comprising:
a carbonyl compound of the formula:

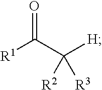

an aryl halide or heteroaryl halide compound of the formula:

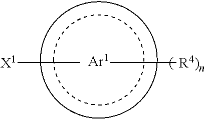

an amine compound;
and a photocatalyst,
under conditions sufficient to produce said α-aryl substituted compound of Formula I,
wherein R¹ is H, alkyl, alkoxyl or aryl;
each of R² and R³ is independently H, alkyl, or aryl; or R¹ and R² together with the atoms to which they are attached to form an optionally substituted cycloalkyl moiety;
Ar¹ is aryl or heteroaryl, each of which is optionally substituted;
n is an integer from 0 to x, where x is a maximum number of substituents possible on Ar¹;

X¹ is Cl, Br, or I; and R^{1a}each R⁴ is independently alkyl, halide, haloalkyl, carbonyl group, cyano, —CO₂R^{a}, —OR^{b}, —NR^{c}R^{d}, an amide, —SR^{a}, —BR^{e}R^{f}, —PR^{e}R^{f} where R^{a} is alkyl, R^{b} is alkyl or a hydroxy protecting group, R^{c} is H or alkyl, and R^{d} is H, alkyl, or an amine protecting group, R^{e} and R^{f} are each independently H, alkyl, hydroxy or an alkoxy group, or W and R f together with the atom to which they are attached to form an optionally substituted cyclic moiety.

In some embodiments, n is 0. In some embodiments, n is 1. In some embodiments, n is 2. In some embodiments, n is 3. In some embodiments, n is 4. In some embodiments, n is 5. In some embodiments, n is 0, 1, 2, 3, 4 or 5.

In some embodiments, the photocatalyst comprises acridinium based photocatalysts such as hexamethoxy acridiniums, a 9-mesityl-acridinium compound; heliceniums; anguleniums; xanthene based photocatalysts such as rhodamine-6G, eosin Y; carbazolyl based photocatalysts such as 4-CzIPN; quinoliniums; benzophenones; quinones; pyryliums; cyanoarenes; perylene diimides; a Ru based photocatalyst such as Ru(bpy)3 compound and known derivatives; an Ir based photocatalyst such as Ir(ppy)₃, [Ir(dtbbpy)(ppy)2]PF6 and known derivatives; or any combination of the foregoing.

In certain embodiments of any of the methods described herein, the electromagnetic radiation is a visible light. In one embodiment of any of the methods described herein, the wavelength of the electromagnetic radiation is from about 440 nm to about 650 nm.

In certain embodiments of any of the methods described herein, the aryl halide has a reduction potential less than or equal to excited state oxidation potential of the photocatalyst.

In certain embodiments of any of the methods described herein, the amount of the photocatalyst used is in the range of from about 0.001 equiv. to about 0.5 equiv., such as from about 0.001 equiv. to about 0.25 equiv., or from about 0.001 equiv. to about 0.1 equiv. relative to the amount of the aryl halide compound.

In certain embodiments of any of the methods described herein, the amount of the amine compound ranges from about 1 equiv. to about 10 equiv., such as from about 1 equiv. to about 5 equiv., or from about 1 equiv. to about 2.5 equiv. relative to the amount of the aryl halide compound.

While the yield of the α-aryl substituted carbonyl compound depends on a wide variety of factors, such as but not limited to, reaction temperature, reaction substrate, photocatalyst, amount of each reagents and/or photocatalyst, wavelength used, reaction solvent, etc., in general, the yield of the α-aryl substituted carbonyl compound produced according to the methods described herein is greater than about 20%, such as greater than about 30%, greater than about 40%, greater than about 50%, greater than about 60%, greater than about 70%, greater than about 75%, greater than about 80%, greater than about 85%., greater than about 90%. or greater than about 95%.

Yet another aspect of the invention relates to a method for producing an isocoumarin compound of the formula:

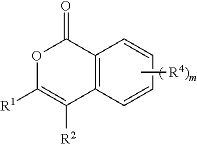

In one embodiment, the method comprises irradiating an electromagnetic radiation to a solution mixture comprising:
a carbonyl compound of the formula:

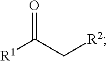

an aryl halide compound of the formula:
an amine compound; and
a photocatalyst,

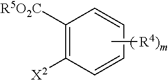

under conditions sufficient to produce said isocoumarin compound,
wherein R¹ is H, alkyl, alkoxyl or aryl;
R² is H, alkyl, or aryl; or R¹ and R² together with the atoms to which they are attached to form an optionally substituted cycloalkyl moiety;
m is an integer from 0 to 4;
X² is Cl, Br, or I;
each R⁴ is independently alkyl, halide, haloalkyl, a carbonyl group, cyano, —CO₂R^{a}, —OR^{e}, —NR^{c}R^{d}, amide, —SR^{a}, —BR^{e}R^{f}, —PR^{e}R^{f} where R^{a} is alkyl, R^{b} is alkyl or a hydroxy protecting group, R^{c} is H or alkyl, and R^{d} is H, alkyl, or an amine protecting group, R^{e} and R^{f} is independently H, alkyl, hydroxy or an alkoxy group or R^{e} and R^{f} together with the atoms to which they are attached to form an optionally substituted cyclic moiety; and
R⁵ is H, alkyl or aryl.

In some embodiments, R¹ and R² together with the atoms to which they are attached to

form an optionally substituted cycloalkyl moiety.

Still in other embodiments, the solution mixture is irradiated with the electromagnetic radiation at a temperature of about 50° C. or less, typically about 40° C. or less, and often about 30° C. or less.

Yet in other embodiments of any of the methods described herein, the electromagnetic radiation is a visible light.

In further embodiments of any of the methods described herein, the photocatalyst comprises acridinium based photocatalysts such as hexamethoxy acridiniums, a 9-mesityl-acridinium compound; heliceniums; anguleniums; xanthene based photocatalysts such as rhodamine-6G, eosin Y; carbazolyl based photocatalysts such as 4-CzIPN; quinoliniums; benzophenones; quinones; pyryliums; cyanoarenes; perylene diimides; a Ru based photocatalyst such as Ru(bpy)3 compound and known derivatives; an Ir based photocatalyst such as Ir(ppy)3, [Ir(dtbbpy)(ppy)2]PF6 and known derivatives; or a combination thereof.

In further embodiments of any of the methods described herein, the photocatalyst comprises Acr6, Acr 7, Acr8, or any combination thereof.

### BRIEF DESCRIPTION OF THE DRAWINGS

FIG. 1 shows photoredox activation for the α-arylation of unactivated cyclic ketones. FIG. 1A shows important bioactive natural products and drug molecules including near-IR dye filter containing α-aryl cyclic ketone core. FIG. 1B shows existing synthetic approaches for the α-arylation of cyclic ketones. FIG. 1C shows one embodiment of the present invention: direct photoredox α-arylation of unactivated cyclic ketones using novel green light acridinium as the photocatalyst. FIG. 1D shows generation of non-trivial compounds from abundant feedstock highlighting the opportunity for cost-effective mild reaction approach for industrial applications.

FIG. 2 shows exemplary development of the photoreducing acridines. FIG. 2A shows synthetic route for the acridiniums. FIG. 2B shows the crystal structure of Acr 7. FIG. 2C shows photophysical properties of the Acr 7. FIG. 2D shows a cyclic voltammogram of Acr 7. FIG. 2E shows a lifetime spectrum of Acr 7.

FIG. 3 shows realization of a photocatalytic α-arylation of unactivated ketones. FIG. 3A shows an exemplary optimization reaction with different light sources, amines and photocatalysts and the crystal structure of the α-arylated ketone product 11. FIG. 3B shows a valuation of different light sources and amines. FIG. 3C shows a valuation and optimization of the photocatalysts or the photoredox α-arylation of cyclic ketones. Yields are calculated from ¹H NMR spectra using 1,3,5-trimethoxy benzene as internal standard. Reactions were run with 5 equiv pyrrolidine under 518 nm green light. LED, light emitting diode.

FIG. 4 shows the reaction scope of photocatalytic α-arylation of unactivated ketones. FIG. 4A shows substrate scope of aryl halides, and isolated yield. FIG. 4B shows substrate scope of ketones, isolated yield. 2 mol % Acr 7 was used. Reaction was run in 50° C.

FIG. 5 shows exemplary synthetic applications. FIG. 5A shows an exemplary large-scale reaction. FIG. 5B shows application to the synthesis of critical pharmaceutical intermediates for bioactive and drug molecules. FIG. 5C shows application to the synthesis of expensive feedstock for clinical candidates from inexpensive starting materials.

FIG. 6A shows a control experiment with commonly used organic photocatalysts and electron-rich acridinium Acr 7. FIG. 6B shows Photo-Arbuzov reaction to probe the SET mechanistic pathway. Equimolar amounts of Acr 7 and aryl halide with excess triethyl phosphite were irradiated in the absence of amine. FIG. 6C shows the spectroelectrochemical spectra of Acr 7 in MeCN. FIG. 6D shows the transient absorption spectra of Acr 7 (2.0 mol %, MeCN, 1 mm path length) in the presence of 4-iodobenzonitrile (1.0 equiv.), cyclohexanone (5.0 equiv.) and pyrrolidine (5.0 equiv.).

FIG. 7 shows a proposed mechanism of the photoredox α-arylation of ketone.

FIG. 8 shows UV-Vis absorption and fluorescence spectra of acridiniums Acr 6, Acr 7 and Acr 8.

FIG. 9 shows cyclic voltammograms of acridiniums Acr 6, Acr 7 and Acr8 .

FIG. 10 shows lifetime spectra of acridiniums Acr 6, Acr 7 and Acr 8.

FIG. 11 shows a reaction trend of the photoredox α-arylation of an α-ketone in the presence and absence of green light.

FIG. 12 shows spectrochemical data collected for Acr 7 in acetonitrile.

FIG. 13 shows transient absorption spectra following the reaction of 4-iodobenznonitrile (9) with cyclohexanone (10) in the presence of Acr 7 in acetonitrile.

FIG. 14 shows a proposed oxidative quenching mechanism cycle, SET (single electron transfer) pathway.

FIG. 15 shows a proposed oxidative quenching mechanism cycle, XAT (halogen atom transfer) pathway.

FIG. 16 shows the X-ray crystallographic molecular structure of Acr 7.

FIG. 17 shows the X-ray crystallographic molecular structure of Acr 8.

FIG. 18 shows the X-ray crystallographic molecular structure of (4-(2-oxocyclohexyl)benzonitrile).

FIG. 19 shows the X-ray crystallographic molecular structure of (4-(8-oxo-1,4-dioxaspiro[4.5]decan-7-yl)benzonitrile).

### DETAILED DESCRIPTION OF THE INVENTION

The present invention provides a method for synthesizing α-arylated carbonyl compounds using a photocatalyst (e.g., a metal free carbocation photocatalyst). The methods of the present invention allow synthesis of a wide range of α-arylated carbonyl compounds at a significantly reduced temperature compared to conventional methods, such as transition metal-based α-arylation (e.g., using a palladium catalyst), thereby avoiding high temperature and the use of toxic and expensive additives. The methods of the present invention can be used to produce a wide variety of fine chemicals as well as biologically active compounds.

In one aspect of the present invention, a method is provided for a green-light-mediated α-arylation of unactivated carbonyl (e.g., unactivated ketone) from readily-available aryl halides via activation of C(sp²)—X bond (X is, for example, I, Br, or Cl) and α-carbonyl C(sp³)—H bond in a single photocatalytic cycle.

The direct α-arylation of carbonyl compounds, in particular unactivated carbonyl compounds, from aryl halides provided in the present disclosure represents a powerful method for the synthesis of a wide variety of building blocks for diverse useful compounds. While numerous conventional synthetic methods exist to forge a C(sp²)—C(sp³) bond, high temperature requirements and/or expensive starting materials limit their use. Some of the advantages of the methods described herein include, but are not limited to, mild reaction conditions (e.g., a reaction temperature of about 50° C. or less, typically about 40° C. or less, or about 30° C. or less), operational simplicity, and wide functional group tolerance. The photocatalytic reaction methods described herein allow for the production of desired compounds in multi-gram scales at a substantially lower reaction time compared to conventional transition metal catalyzed reactions. The methods described herein can be used, e.g., to produce a wide variety of feedstock chemicals or starting materials or intermediates that are commercially expensive but critical for synthesizing numerous pharmaceutical agents.

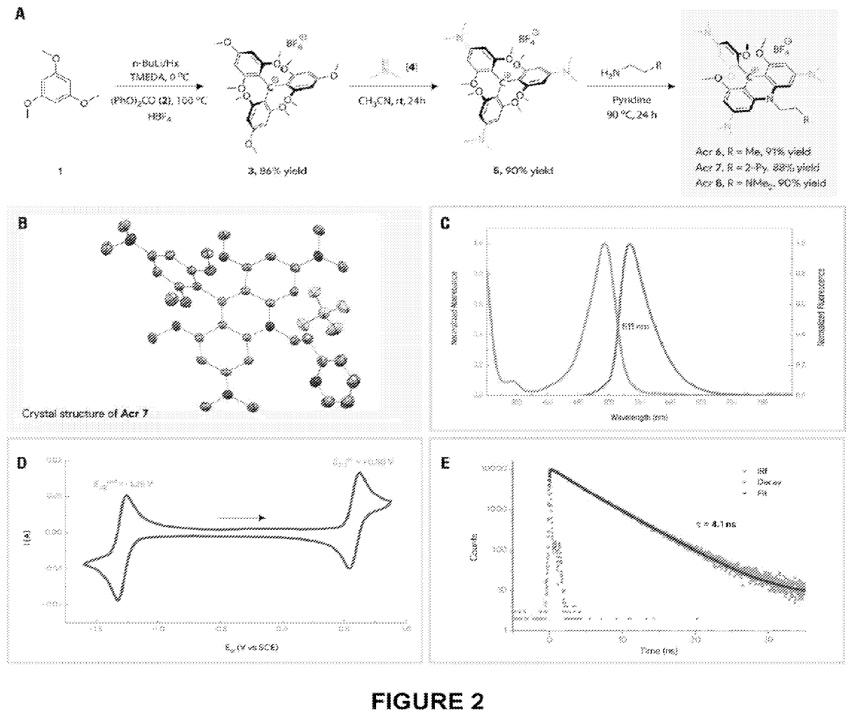

Over the past several decades, photoredox catalysis promises to be a powerful, clean and sustainable synthetic method in organic synthesis. Accordingly, photoinduced electron transfer (PET) has emerged as an alternative to heavy-metal based catalytic reactions. The photoredox reaction provides access to previously inaccessible substrates, thereby fostering the use of abundant and inexpensive starting materials. Of particular interest is producing α-arylated carbonyl compounds via a visible-light-mediated process using benchtop carbonyls and aryl halides. Conventional photoredox reactions have demonstrated impressive progress in variety of α-alkylation to the carbonyl compounds. However, one of the key limitations in conventional photoredox α-arylation of carbonyl compounds is the requirement of activated ketones such as α-chlorocarbonyls and α-phenylselanylketones (FIG. 2B). Despite the noteworthy advancement in photocatalysis to activate C(sp²)—X bond and α-carbonyl C—H bond, to date photoredox α-arylation to prepare carbonyls from aryl halides and unactivated carbonyl compound have not been achieved.

The present inventors have surprisingly found that visible light can be used with a variety of photoredox catalysts in the presence of an amine compound to readily provide α-arylation of carbonyl compounds, including unactivated carbonyl compounds.

As used herein, the term “unactivated carbonyl compound” refers to a carbonyl compound in which the desired reaction site, i.e., the α-position of the carbonyl carbon, does not contain an electron withdrawing group. In one embodiment, the α-position of the unactivated carbonyl compound contains hydrogen atoms.

As used herein, the term “unactivated ketone” refers to a ketone compound in which the desired reaction site, i.e., the α-position of the carbonyl carbon, does not contain an electron withdrawing group. In one embodiment, the α-position of the unactivated ketone contains hydrogen atoms.

As used herein, the term “carbonyl compound” refers to ketones and aldehydes. In one embodiment, the carbonyl compound is a ketone. The term “unactivated carbonyl compound” generally refers to a carbonyl compound in which α-carbon to the carbonyl carbon are substituted with hydrogen or unsubstituted alkyl group. Typically, “unactivated carbonyl compound” does not have any atom on the α-carbon of the carbonyl carbon that has electronegativity that is about 0.5 or higher, such as 0.3 or higher, or 0.1 or higher than hydrogen. Exemplary atoms having an electronegativity higher than carbon include, but are not limited to, halide, heteroatoms such as O, N, and S, functional groups such as —CN, as well as others atoms or functional groups that are known to one skilled in the art as being a carbonyl “activating group”, aryl and alkyl selanyl (SeR), and an additional carbonyl group (e.g., diketones such as acetyl acetonate).

As used herein, the term “secondary amine compound” refers to an amine compound in which the nitrogen atom of the amine functional group is attached to two carbon atoms (i.e., two carbon containing compounds) and one hydrogen atom. The carbon containing compounds can independently be, for example, any C₁-C₁₂ alkyl group, or can form a 3-, 4-, 5-, 6-, 7-, or more membered mono- or bi-cyclic structure with the nitrogen atom to which they are attached.

The term “cyclic carbonyl compound” refers to a non-aromatic, typically saturated, monovalent mono- or bicyclic hydrocarbon carbonyl compound of three to twelve ring carbons optionally having one or two heteroatom, such as O, N, or S, in the ring structure. When the heteroatom is present in the ring structure, such a heteroatom is not attached to the carbonyl carbon. The cyclic carbonyl compound can be optionally substituted with one or more, e.g., one, two, or three, substituents within the ring structure. When two or more substituents are present in a cyclic carbonyl compound, each substituent is independently selected. It should be appreciated that in “unactivated cyclic carbonyl compound” the substituent, if present, is at a position other than the α-position of the carbonyl carbon. Exemplary substituents that may be present in cyclic carbonyl compound include, but are not limited to, alkyl, esters, ethers, ketal, protected hydroxyl group, CN, fluorinated alkyl, aryl, cyclic alkyl or cyclic aryl. It should also be noted that the cyclic carbonyl compound can include fused ring systems, where the fused ring can be another cyclic alkyl or aryl.

The term “alkyl” refers to a saturated linear monovalent hydrocarbon moiety of one to twelve, typically one to six, carbon atoms or a saturated branched monovalent hydrocarbon moiety of three to twelve, typically three to six, carbon atoms. Exemplary alkyl group include, but are not limited to, methyl, ethyl, n-propyl, 2-propyl, tent-butyl, pentyl, and the like.

The term “ester” refers to a moiety of the formula: —CO₂R, where R is alkyl as defined herein.

The term “ether” refers to a moiety of the formula: —OR, where R is alkyl as defined herein.

The term “ketal” refers to a cyclic moiety of the formula —O—R—O—, where R is alkylene.

The term “alkylene” refers to a linear saturated divalent hydrocarbon moiety of two to six carbon atoms or a branched saturated divalent hydrocarbon moiety of three to six carbon atoms, e.g., ethylene, propylene, 2-methylpropylene, pentylene, and the like.

The term “protected hydroxy group” refers to a moiety of the formula —OR^{a}, wherein W is a hydroxy protecting group. Examples of hydroxy protecting groups can be found in, for example, T. W. Greene and P. G. M. Wuts, Protective Groups in Organic Synthesis, 3^{rd} edition, John Wiley & Sons, New York, 1999, and Harrison and Harrison et al., Compendium of Synthetic Organic Methods, Vols. 1-8 (John Wiley and Sons, 1971-1996), which are incorporated herein by reference in their entirety. Representative hydroxy protecting groups include acyl groups, benzyl and trityl ethers, tetrahydropyranyl ethers, trialkylsilyl ethers and allyl ethers, and the like.

The term “haloalkyl” refers to an alkyl group as defined herein in which one or more hydrogen atom is replaced by the same or different halogen atom(s). The term “haloalkyl” also includes perhalogenated alkyl groups in which all alkyl hydrogen atoms are replaced by halogen atoms. Exemplary haloalkyl groups include, but are not limited to, —CH₂Cl, —CF₃, —CH₂CF₃, —CH₂CCl₃, and the like.

The terms “halo,” “halogen” and “halide” are used interchangeably herein and refer to fluoro, chloro, bromo, or iodo, typically chloro, bromo, or iodo.

The term “alkoxy” refers to a moiety of the formula —OR, where R is alkyl or haloalkyl as defined herein. Typically, R is alkyl.

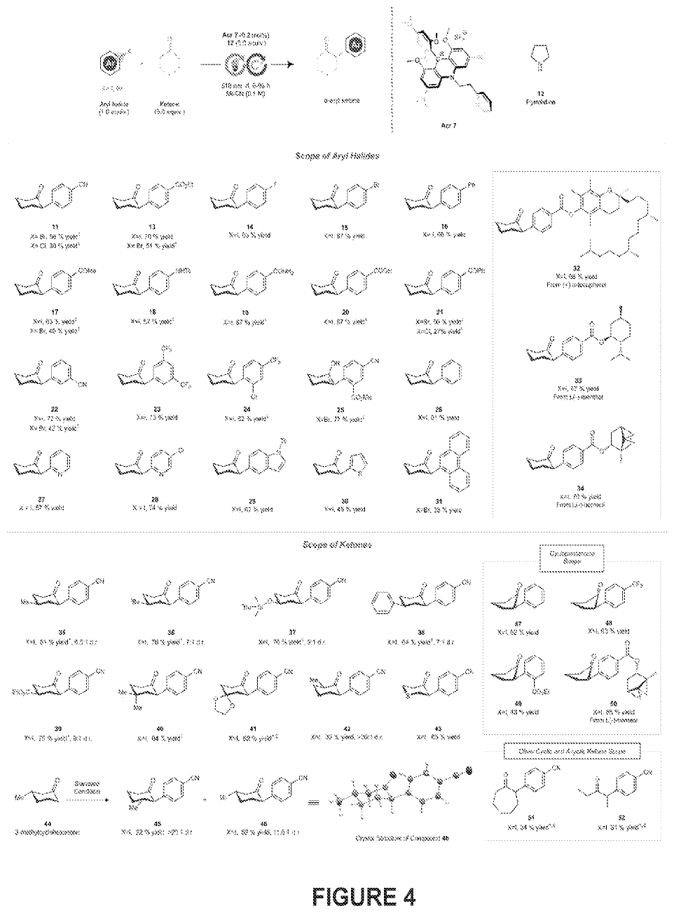

The term “aryl” refers to a monovalent mono-, bi- or tricyclic aromatic compound of 6 to 15 ring atoms which is optionally substituted with one or more, typically one, two, or three substituents within the ring structure. It should be noted the aryl moiety can also be connected to other organic moieties such as those shown in compounds 36-38 and 54 in FIG. 4. When two or more substituents are present in an aryl group, each substituent is independently selected. Exemplary substituents for the aryl group include, but are not limited to, complex organic moieties as shown in compounds 36-38 and 54 in FIG. 4, alkyl, haloalkyl, thioalkyl, heteroalkyl, halo, nitro, cyano, cycloalkyl, optionally substituted phenyl, heteroaryl, heterocyclyl, haloalkoxy, optionally substituted phenyloxy, heteroaryloxy, —COR (where R is alkyl or optionally substituted phenyl), -(alkylene)ₙ—COOR (where n is 0 or 1 and R is hydrogen, alkyl, optionally substituted phenylalkyl, or heteroaralkyl), or —(alkylene)n—CONR^{a}R^{b} (where n is 0 or 1, and R^{a} and R^{b} are, independently of each other, hydrogen, alkyl, cycloalkyl, cycloalkylalkyl, hydroxyalkyl, aryl, or R and R′ together with the nitrogen atom to which they are attached form a heterocyclyl ring).

The term “heteroaryl ring” refers to a group in which one or more of the aromatic ring carbon atoms is a replaced with a heteroatom, each of which may be independently selected. Exemplary heteroatoms in heteroaryl ring include, but are not limited to, O, N, and S. Exemplary heteroaryl rings include, but are not limited to, pyridyl, furanyl, thiophenyl, thiazolyl, isothiazolyl, triazolyl, triazinyl, imidazolyl, oxazolyl, isoxazolyl, pyrrolyl, pyrazolyl, pyrazinyl, pyrimidinyl, benzofuranyl, isobenzofuranyl, benzothiazolyl, benzoisothiazolyl, benzotriazolyl, indolyl, isoindolyl, benzoxazolyl, thiazolyl, isothiazolyl, quinolyl, isoquinolyl, benzimidazolyl, benzisoxazolyl, benzothiophenyl, dibenzofuran, and benzodiazepin-2-one-5-yl, and the like.

Some aspects of the present invention are based on the discovery that an organic photocatalyst can activate both the α-carbonyl C—H bond and C(sp²)—X bond in a single photocatalytic cycle which leads to a simple, mild and clean organic transformation. In one particular embodiment, an acridine compound (sometime referred herein as “acridinium” to indicate its positive ionic nature with a corresponding anionic pair) with a photoredox window wide enough to access both the C(sp²)—X bond of aryl halides and the α-carbonyl C—H bond in unactivated cyclic ketones can be used. Notably, this acridinium is an organic photocatalyst acting as a photo-reductant involving oxidative quenching process.

In one embodiment, the present invention generally relates to methods and compositions for producing α-aryl carbonyl compounds from a carbonyl compound, aryl halide, and a photocatalyst in the presence of an amine compound. Methods and compositions of the present invention are particularly useful in producing α-aryl substituted cyclic ketones. More particularly, methods and compositions of the present invention are useful in producing α-aryl substituted cyclic ketones from unactivated cyclic ketones. For the sake of clarity and brevity, the present invention will now be described in detail with reference to producing α-aryl substituted cyclic ketones from unactivated cyclic ketone compound. However, it should be appreciated that the scope of the invention is not limited to using an unactivated cyclic ketone. In fact, as stated above, methods and compositions of the present invention can be used to produce α-aryl substituted carbonyl compounds from any carbonyl compound, including acyclic carbonyl compounds. A detailed discussion on producing α-aryl cyclic ketone from unactivated ketone is provided solely for the purpose of illustrating the practice of the invention and do not constitute limitations on the scope thereof

Therefore, in one embodiment, disclosed herein is a light-mediated (e.g., green-light-mediated) acridinium-catalyzed direct α-arylation of unactivated ketones with aryl halides for the synthesis of α-aryl carbonyl compounds under mild reaction conditions (Scheme 1).

It should be appreciated that any photocatalyst can be used in the methods of the present invention. Exemplary photocatalysts that are suitable in any of the methods disclosed herein include, but are not limited to, acridinium based photocatalysts such as hexamethoxy acridiniums, a 9-mesityl-acridinium compound; heliceniums; anguleniums; xanthene based photocatalysts such as rhodamine-6G, eosin Y; carbazolyl based photocatalysts such as 4-CzIPN; quinoliniums; benzophenones; quinones; pyryliums; cyanoarenes; perylene diimides; a Ru based photocatalyst such as Ru(bpy)3 compound and known derivatives; an Ir based photocatalyst such as Ir(ppy)3, [Ir(dtbbpy)(ppy)2]PF6 and known derivatives; or any combination of any of the foregoing.

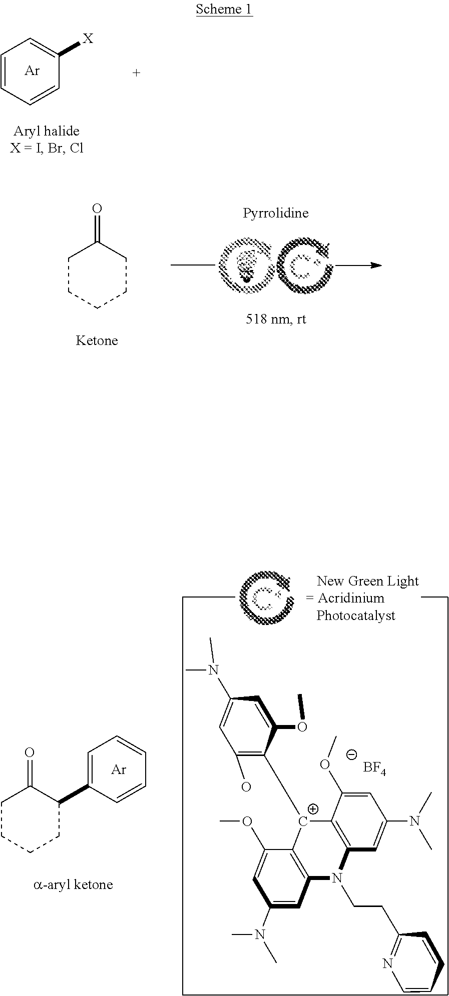

In some embodiments of any of the methods described herein, the photocatalyst comprises an acridinium of formula (I):

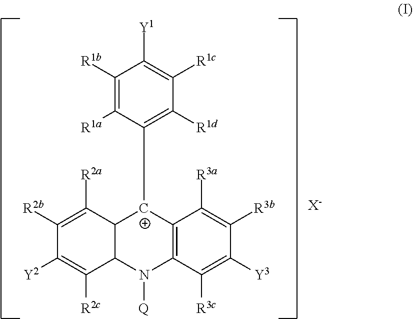

wherein
each of R^{1a}, R^{1ab}, R^{1c}, R^{1d}, R^{2a}, R^{2b}, R^{2c}, R^{3a}, R^{3b} and R^{3c} is independently H, halide, haloalkyl (e.g., CF3), —NR^{a}R^{b}, C₁-C₁₂ alkyl, C₁-C₄ alkoxy, —NO₂, —CN, —CO₂R (wherein R is H or C₁-C₄ alkyl), or Ar¹, wherein each of R^{a} and R b is independently H or ₁-C₄ alkyl;
each of Y¹, Y² and Y³ is independently —NR^{4a}R^{4b}, OR^{4a}, or R^{4a}, wherein each of R^{4 a} and R^{4b} is independently C1-C12 alkyl, haloalkyl (e.g., CF3), or aryl;
Q is C1-C12 alkyl, a moiety of the formula —L—Z, wherein L is C2-C12 alkylene, and Z is amino, monoalkylamino, dialkylamino, or pyridyl; and
X⁻ is a counter anion.

The acridinium cations described herein themselves do not necessarily comprise chemical compounds. Indeed, in an isolable compound, cations must be paired with a corresponding anion to maintain electroneutrality. Thus, while the present disclosure may sometimes refer to acridinium of

Formula I without indicating the X⁻ anion, it should be appreciated that such a notation is a short-handed notation of Formula I that includes the counter anion. In general, the nature of X⁻ is not important as long as electroneutrality is maintained. Exemplary X⁻ of an acridinum of Formula I may include, but is not limited to, halide, BF4⁻, sulfate, sulfite, phosphate, phosphite, carboxylate (e.g., acetate), alkoxide, hydroxide, triflate, PF6⁻, borate, fluorinated borate, and other suitable anions known to one skilled in the art. It should be appreciated that the scope of the invention is not limited to having X⁻ be those disclosed above. In general, any X⁻ known to one skilled in the art can be used. It will be appreciated that a molar ratio of an anion to a acridinium cation of the present invention depends on the valence of the cation. Thus, if both the cation and the anion are monovalent, then the ratio will be a 1:1 molar ratio between the cation and the anion. Whereas if the anion is divalent, then there will be a 1:2 molar ratio between the anion and the acridinium cation.

In other embodiments of any of the methods disclosed herein, the photocatalyst can include acridinium, helicenium, angulenium, or any combination thereof. See, e.g., International Publication No. WO 21/155331, which is incorporated herein by reference in its entirety.

The amount of photoredox catalyst (photocatalyst) used in any of the methods of the present invention can vary depending on a variety of factors, such as reaction solvent, reaction temperature, the concentration of each materials used, etc. Typically, the amount of photoredox catalyst used ranges from about 0.001 equiv. to about 0.5 equiv., such as from about 0.001 equiv. to about 0.1 equiv., or from about 0.001 equiv. to about 0.05 equiv. relative to the amount of said aryl halide. However, it should be appreciated that the scope of the invention is not limited to these particular ranges. Throughout this disclosure, when referring to a numerical value, the terms “about” and “approximately” are used interchangeably herein and refer to being within an acceptable error range for the particular value as determined by one of ordinary skill in the art. Such a value determination will depend at least in part on how the value is measured or determined, e.g., the limitations of the measurement system, i.e., the degree of precision required for a particular purpose. For example, the term “about” can mean within 1 or more than 1 standard deviation, per the practice in the art. Alternatively, the term “about” when referring to a numerical value can mean±20%, typically ±10%, often ±5% and more often ±1% of the numerical value. In general, however, where particular values are described in the application and claims, unless otherwise stated, the term “about” means within an acceptable error range for the particular value, typically within one standard deviation.

The reaction temperature in any of the methods of the present invention can also vary depending on a variety of factors such as reaction solvent, the concentration of each materials used, etc. However, in certain embodiments, the reaction temperature ranges from about 0° C. to about 100° C. (or to the boiling point of the solvent used), such as about 10° C. to about 80° C., or about 20° C. to about 50° CIN one embodiment, the reaction temperature is at or near ambient temperature (e.g., 20° C-25° C.). However, it should be appreciated that the scope of the invention is not limited to these particular temperature ranges and can include higher and/or lower temperatures.

Generally, the reaction is conducted in an organic solvent. The organic solvent can comprises a single organic solvent, or it can be a mixture of two or more organic solvents. Exemplary organic solvents that can be used in any of the methods of the present invention include, but are not limited to, ethers such as diethyl ether and tetrahydrofuran, acetonitrile, benzene, toluene, xylene, acetone, DMF, NMP, DMSO, and alcohols, diglyme, as well as other non-halogenated organic solvents known to one skilled in the art.

The methods of the present invention typically use an excess of the cyclic carbonyl compound relative to the aryl halide or heteroaryl halide compound. Typically, the molar equivalent ratio between the amount of cyclic carbonyl compound relative to the aryl halide or heteroaryl halide compound. ranges from about 20:1, such as from about 10:1, or from about 5:1.

The wavelength that can use used to effect the photoredox catalytic reaction in any of the methods described herein can be tuned by varying one of more of the substituents (e.g., R^{1a}—R^{1d}, R^{2a}—R^{2c}, Y², R^{3a}—R^{3c} or Y³) attached to acridine compound of Formula I. In some embodiments, the wavelength used for photocatalytic reaction ranges in all infrared to UV light, typically, in all visible wavelengths, such as from about 440 nm to about 650 nm, from about 440 nm to about 600 nm, from about 440 nm to about 550 nm, or from about 440 nm to about 500 nm. In one embodiment, the wavelength is about 450 nm.

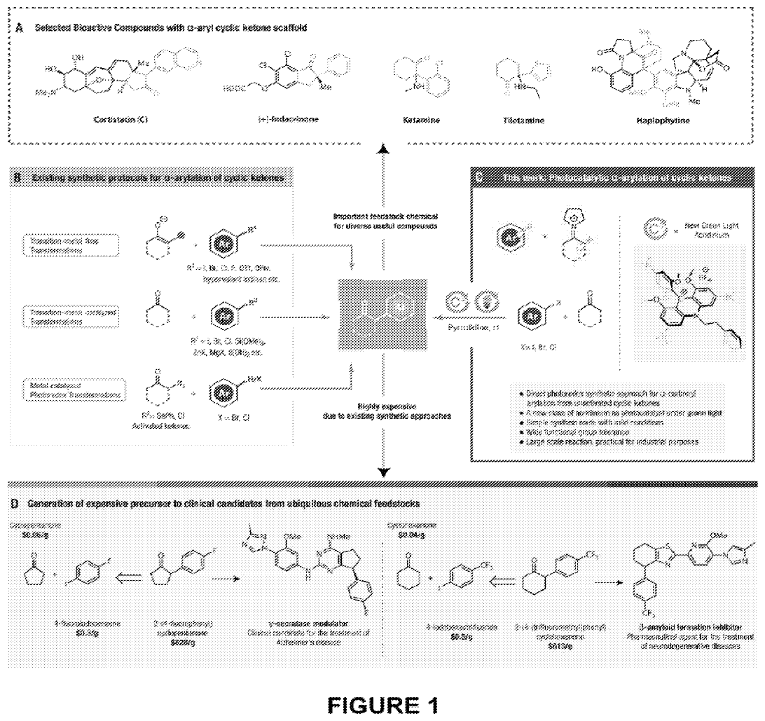

It was discovered by the present inventors that green light in the visible light region contains less energy but have better penetrability to the reaction media, thereby allowing for a smoother transition to the scaled-up reactions viable for industrial applications. Given the ubiquity of α-aryl cyclocarbonyls in bioactive and pharmaceutical molecules, use of the claimed methods for the facile generation of critical but expensive pharmaceutical intermediates from abundantly available and inexpensive starting materials (e.g., FIG. 1D) was envisaged

Recently, acridinium ions (e.g., 9-mesityl-10-methylacridinium) have been proven to be one of the most powerful organic photocatalysts and have been applied in catalyzing a wide range of exquisite transformations under visible light. However, most ofthe cases, they serve as photooxidants through reductive quenching process, and very limited examples in photoreduction catalysis have been reported.

The present inventors have surprisingly discovered that, for example, introducing electron-donating groups to the para position of the aryl group(s) of e.g., tetramethoxy acridinium ions promotes their photoreductive ability. Using methods discovered by the present inventors, a series of electron-rich acridiniums (Acr 6, Acr 7 and Acr 8) was synthesized from 1,3,5-trimethoxybenzene in three steps in good yield, via the intermediate of the two carbocations 3 and 5 (see FIG. 2A).

The structure of Acr 7 has been confirmed by its X-ray crystallographic analysis. Additionally, the UV-Vis absorption spectra of Acr 6-8 showed a red shifted maximum absorption to the green light region (λmax=494 nm) compared to reported acridinium ions. Furthermore, electrochemical studies revealed a reversible reduction event at −1.28 V versus the saturated calomel electrode (SCE) on their cyclic voltammetric (CV) curves, with an additional reversible oxidation event at +0.58 V, which confirms the assumption of applying them in photoreduction catalysis involving oxidative quenching process feasible in theory. The estimated excited-state oxidation (E_{1/2}(C^{108 ++}/C⁺*)) and reduction potential (E½(C⁺*/C^{⋅})) were calculated for this acridinium series, which are at −1.85 V and +1.15 V vs. SCE, respectively. Surprisingly, the different pendant arms within this system ⁿPr for Acr 6, (CH₂)₂Py for Acr 7, and (CH₂)₂ NMe₂ for Acr 8 have very little influence on their electrochemical properties. Moreover, the excited-state lifetime (τ=3.85, 4.10, 2.65 ns for Acr 6, 7 and 8, respectively) is also comparable to the values of other common organic photocatalysts (2-20 ns in general). Thus, it was predicted that Acr 6-8 should be able to reduce the aryl halides with the reduction potential <−1.85 V vs SCE to generate the corresponding aryl radicals through oxidative quenching under visible light. Given that the oxidation potential of enamines is lower than +0.5 V vs. SCE and can be generated in situ by reacting a cyclic carbonyl compound with a secondary amine, it was believed that the oxidative quenching process of Acr 6-8 (E_{1/2}(C^{⋅++}/C⁺)=+0.58 V vs SCE) is compatible with enamines. Therefore, the acridinium family Acr 6-8 should be competent to act as an efficient organic photocatalyst for catalyzing the direct α-arylation of cyclic ketones with aryl halides in the presence of a secondary amine under green light, which allows for a general and practical access to α-aryl carbonyl compounds under mild conditions.

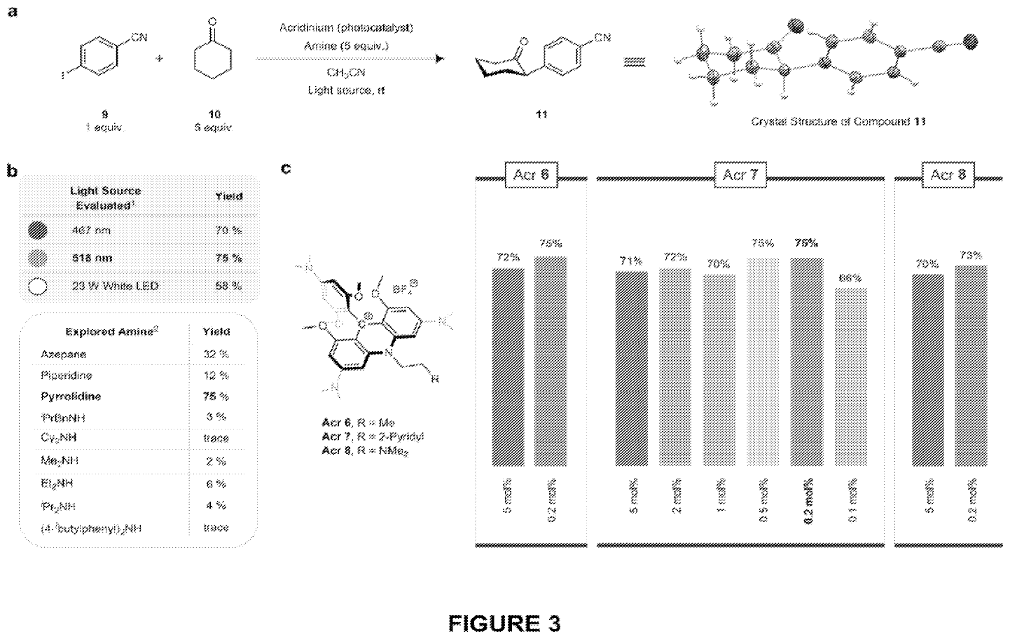

To develop a photocatalytic reaction protocol, a reaction between 4-iodobenzonitrile 9 and cyclohexanone 10 was initially examined. See FIG. 3A.

Surprisingly and unexpectedly, the initial reaction proceeded smoothly in acetonitrile (MeCN) under green light (λmax=518 nm) in the presence of Acr 7 (5 mol %) by using pyrrolidine (14) to generate the enamine in situ, which afforded the desired product 11 in 70% NMR yield. Running the reaction under blue (λmax=467 nm) or white light (23 W) produced 11 in 70% and 60% NMR yield, respectively (FIG. 3B). Thus, follow-up optimizations were conducted under green light (λmax=518 nm), which also has a relatively low energy and higher penetration depth. The investigation of various amines showed that the reaction with pyrrolidine 12 resulted in the highest NMR yield (75%) among the different cyclic and acyclic secondary amines evaluated (FIG. 3B). Thus, pyrrolidine 12 was chosen as the amine for further study. Testing of catalyst loading of Acr 7 suggested that both 0.2 mol % and 0.5 mol % gave the best performance, furnishing (11) in 75% NMR yield (FIG. 3C). Reactions of 9 with 10 in the presence of pyrrolidine 12 by employing Acr 6 or 8 as the photocatalyst yielded similar outcomes compared to Acr 7, which is not surprising since all the three acridiniums share almost indistinguishable photophysical and electrochemical properties. Some dehalogenated product was observed as a byproduct in the crude ¹H NMR spectra. The amount decreased with lower catalyst loading improving the overall yield for the desired α-arylated product. Thus, the use of MeCN as the solvent, pyrrolidine 12 as the amine, Acr 7 as the photocatalyst, 0.2 mol % as the catalyst loading, and green light (λmax=518 nm) as the light source were used in further studies.

With the conditions identified above, the scope of green-light-mediated direct α-arylation protocol was explored. Using cyclohexanone as the model ketone substrate, different aryl halides were tested. As shown in FIG. 4, a range of different para, meta and ortho substituted aryl halides were well-tolerated (13 to 25, 40 to 77% yield), as was unsubstituted iodobenzene (26, 61% yield). Heteroaryl iodides also performed well, smoothly furnishing the desired α-arylated products with different heteroaromatic rings such as pyridine (27 and 28, 57 and 74% yield), indole (29, 62% yield) and thiophene (30, 45% yield). Additionally, aryl bromides bearing a variety of functional groups such as cyano (11 and 22, X=Br, 56% and 42% yield), ester (13, X═Br, 51% yield), ketone (17 and 29, X=Br, 40% and 50% yield), and 9-phenanthrene (31, 33% yield) were well-tolerated, giving rise to the expected α-aryl ketones, albeit in slightly lower yields compared to aryl iodides. Moreover, we also evaluated the competence of aryl chlorides. The corresponding α-arylcyclohexanone was indeed formed, although in low yield (11 and 21, X=Cl, 30% and 27% yield), presumably due to the stronger bond dissociation energy of the C(sp²)—Cl bond (C(sp²)—Cl>C(sp²)—Br>C(sp²)—I)⁴⁰. Nevertheless, the utility of this method was further strengthened by three complex chiral aryl iodide substrates that have been synthesized from (+)-α-tocopherol, (−)-menthol, and (−)-borneol, accordingly. These aryl iodides from natural products were demonstrated to be excellent substrates for this protocol (32 to 34, 66 to 70% yield). It is noteworthy to mention that, a wide range of useful functional groups including nitrile (11, 22), ester (13), halides (14, 15, 24), ketone (17, 21), tosylamine (18), amide (19), carboxylic acid (20) and trifluoromethyl (23) proved to be compatible with this reaction manifesting the versatility and practicability of the present invention.

Cyclohexanones with different electron-donating or electron-withdrawing groups at 4-position performed well, smoothly furnishing the corresponding α-aryl cyclic ketone with useful cis-diastereocontrol (35 to 39; 64% to 81% yield with 6.5:1 to 9:1 d.r.). Additionally, disubstitutions at the 4-position such as gem-dimethyl (40; 64% yield) and ketal (41; 62% yield) were also well-tolerated. However, elevated temperature (50° C.) was required to synthesize 41, presumably due to the slow formation of intermediate enamine at room temperature. The reactions for 4-substituted cyclohexanones were performed with 2 mol % catalyst loading as it demonstrated better yield over 0.2 mol % catalyst loading. On the other hand, under optimal reaction condition, 2-methyl cyclohexanone formed the desired α-arylated product with an excellent cis-diastereoselectivity, albeit in a compromised yield possibly due to steric effect (42; 32% yield, >20:1 d.r.). Moreover, when 3-methylcyclohexanone 44 was evaluated under optimal condition, two regio-isomeric products were isolated with good diastereoselectivity (45 and 46; 22% yield, >20:1 d.r. and 52% yield, 11.5:1 d.r.). Heterocyclic ketones such as tetrahydro-4H-thiopyran-4-one was also demonstrated to be compatible, affording the corresponding product in good yield (43; 65% yield). As a different ring sized cyclic ketone scope, cyclopentanone also engaged productively with iodobenzene, 4-iodobenzotrifluoride and differentially substituted aryl esters (47 to 50; 43% to 65% yield). However, the in situ enamine formation of cycloheptanone was found to be not as facile as cyclohexanones and cyclopentanone⁴². Therefore, we attempted the reaction at 50° C. and observed 34% product (51) formation after 96 h. Similar behavior was also observed in case of acyclic ketone like pentanone (52, 31% yield).

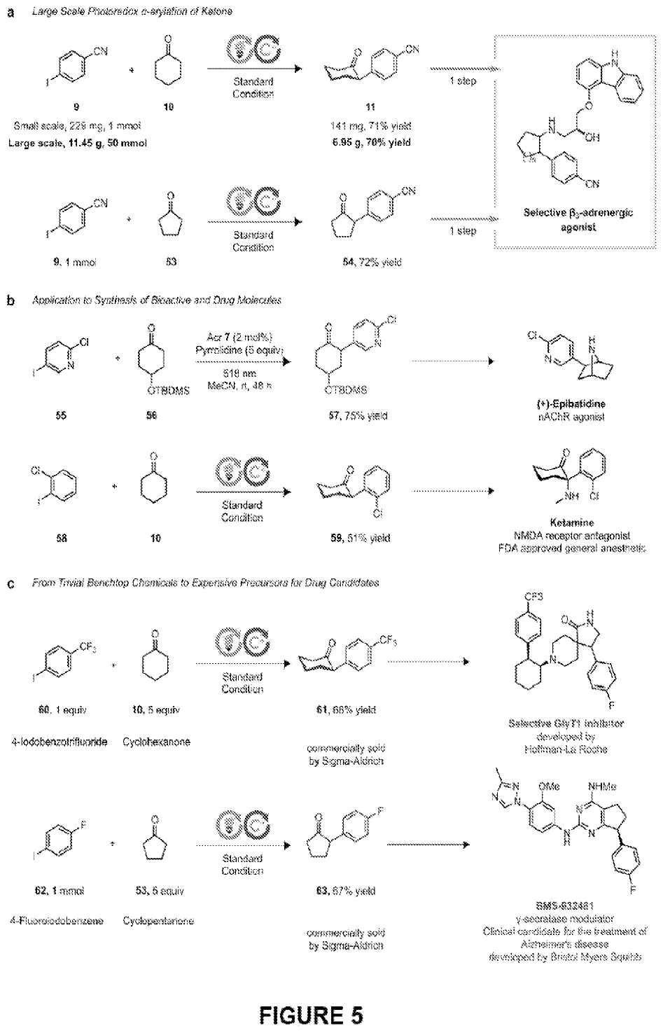

Since green light has better penetrability into the reaction media compared to blue light, green light was used to produce multigram scale synthesis to demonstrate industrial applicability of methods of the invention. Accordingly, a large-scale reaction was performed to synthesize 11, a precursor for a selective β3-adrenergic agonist (FIG. 5A). As can be seen, the synthesis of 11 from 4-iodobenzonitrile and cyclohexanone, on a 50 mmol scale under standard conditions resulted in a similar outcome as the 1 mmol scale reaction (11, 70% and 71% yield in 50 mmol and 1 mmol, respectively). This demonstrates the overall strength and practicality of methods of the present invention to form α-arylated cyclocarbonyl compounds in a single step from inexpensive, readily-accessible starting materials. Additionally, the precursor for the cyclopentane variant of the selective β3-adrenergic agonist was synthesized in good yield (54, 72% yield, FIG. 5A).

Inspired by the potential usefulness of this protocol, the utility of this photocatalytic method was evaluated in the formal syntheses of bioactive and drug molecules. In one embodiment, a synthesis of the naturally occurring alkaloid (+)-epibatidine, an nAChR agonist, was performed (FIG. 5B). Using the methods of the present invention, the desired scaffold or intermediate was prepared with a milder reaction condition relative to conventional methods in similar yield (57, 75% yield). Another target compound ketamine, an FDA approved general anesthetic. There are variety of synthetic routes to prepare ketamine in the literature, but the α-arylated cyclohexanone precursor 59 requires multiple steps to synthesize in most cases.

The methods of the present invention were used in a gram scale reaction starting from readily available 2-iodochlorobenzene 58 and cyclohexanone 10 which led to the isolation of the ketamine precursor in moderate yield (59, 51% yield, FIG. 5B). The simplistic nature of this reaction, efficiency and ease of operation were further confirmed through its application in the syntheses of two critical feedstock chemicals for clinical candidates (FIG. 5C). Readily-available 4-iodobenzotrifluoride 60 and cyclohexanone 10 were used in methods of the present invention, resulting in the corresponding α-aryl ketone scaffold in good yield (61, 68% yield). Significantly, 61 is central to the synthesis of a family of CNS active molecules that are under study by Hoffmann La Roche (e.g., 4-substituted-8-(2-phenyl-cyclohexyl)-2,8-diazaspiro[4.5]decan-1-ones) and have been reported to possess antidepressant, anesthetic and analgesic properties. Finally, 2-phenylcyclopentanone 63 was synthesized from regular benchtop reagents such as, 4-fluoroiodobenzene 62 and cyclopentanone 54 following the same general method (63, 67% yield). This compound is an important intermediate or starting material in synthesis of BMS-932481 a γ-secretase modulator that is also a clinical candidate for the treatment of Alzheimer's disease. While both of these scaffolds 61 and 63 are commercially available, but are prohibitively expensive (61, $613 per gram; 63, $628 per gram). The methods of the present invention offer a new, inexpensive but powerful method to develop critical, value-added chemical scaffolds from abundant trivial starting material.

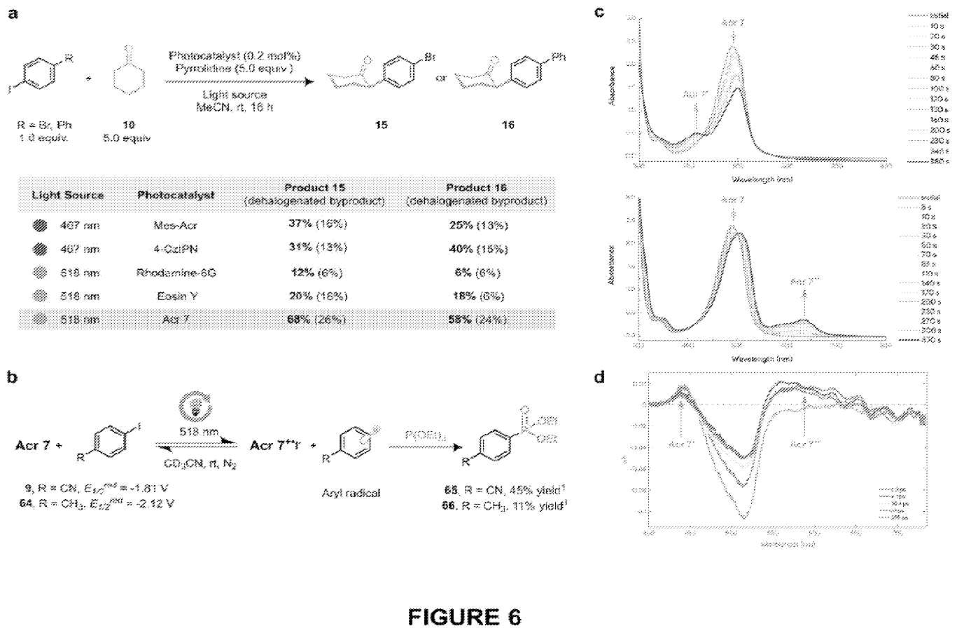

Subsequent efforts were directed towards understanding the photoredox activated mechanism (FIG. 6). Initial control experiments showed that in absence of a light source, or the photocatalyst Acr 7 or the additive pyrrolidine 12, no reaction was observed. Furthermore, during an alternating light/dark experiment reaction progress was observed to be stalled in absence of green-light irradiation. That summarized that light source, photocatalyst Acr 7 and pyrrolidine are integral parts of this method. We then evaluated the performance of some commonly used organic photocatalysts such as, Mes-Acr, 4-CzIPN etc. in this reaction methodology (FIG. 6a). In spite of the fact that these photocatalysts have unmatched photoexcited potentials to reduce aryl halides, formation of α-aryl ketone was observed in low yields. This indicates the existence of XAT as viable mechanistic pathway for the traditional photocatalysts. However, in context of C(sp²)—X bond activation, these catalysts were all outperformed by our acridinium photocatalyst Acr 7 (FIG. 6a). That substantiates the evidence of SET pathway contributing to the XAT mechanism as both are accessible and have matched photoexcited potentials in case of Acr 7. These results benchmarked the photocatalytic efficiency of this acridinium family Acr 6-8. Next, we attempted the photo-Arbuzov reaction in absence of amine to exclusively observe the existence of SET mechanism (FIG. 6b). Konig and colleagues reported triethyl phosphite as a fast-coupling partner for in situ formed aryl radical in photo-Arbuzov reaction⁶¹. Therefore, absence of amine during this reaction makes it a suitable transformation to probe the SET pathway. Thus, we performed the photo-Arbuzov reaction using triethyl phosphite, Acr 7 under 518 nm green LED, and two aryl halides with different redox potential (FIG. 6b, 4-iodobenzonitrile 9: E_{1/2}^{red}=−1.81 V vs SCE and 4-iodotoluene 64: E_{1/2}^{red}=−2.12 V vs SCE). In both cases the aryl-phosphite adduct was observed, despite the absence of pyrrolidine or enamine (65 and 66, 45% and 11% NMR yield). Hence, it strongly supports the hypothesis of aryl radical generation via SET between the excited state Acr 7 and the aryl halide. In order to gain further insights, transient absorption spectra of the model reaction were recorded (FIG. 6d) and compared to the spectroscopic signature of Acr 7^{⋅} and Acr 7^{+⋅} obtained from the spectroelectrochemical study of the photocatalyst Acr 7 in MeCN (FIG. 6c). Prominent excited state absorption signals were apparent for both of the Acr 7¹⁰⁸ and Acr 7^{30 ⋅} confirming our initial assumption of concurrent SET and XAT contribution to this reaction method. Nevertheless, further study to unfold detailed kinetic insights of each mechanistic paths are ongoing.

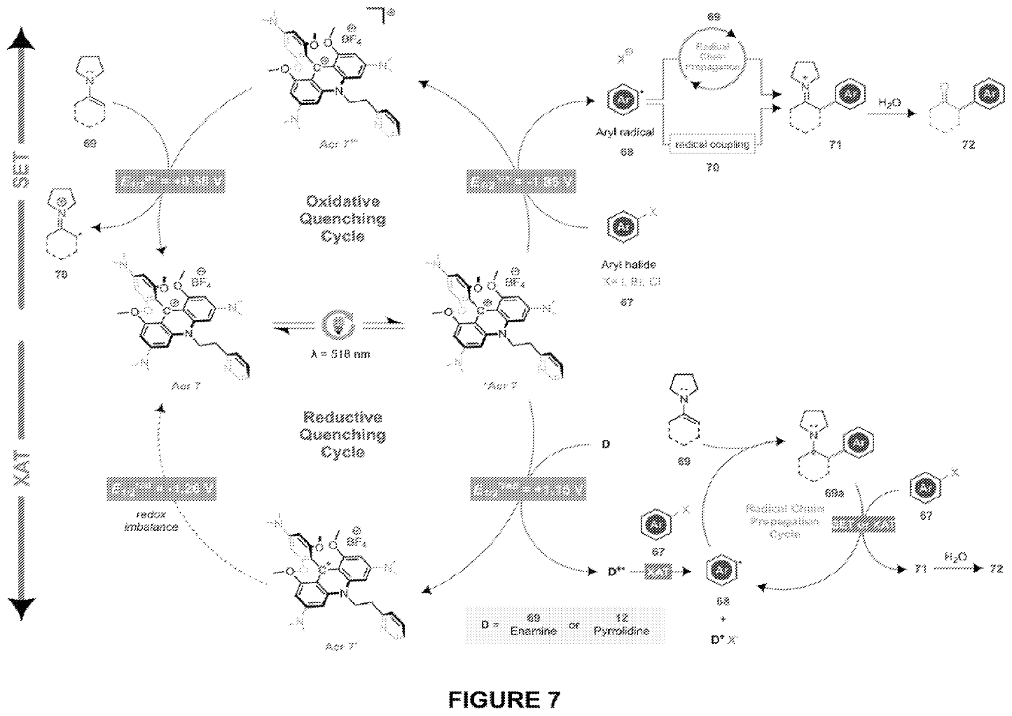

Without being bound by any theory, a prospective mechanism involving both SET and XAT pathway is presented here (FIG. 7). We propose that in presence of a green light source (λ=518 nm) Acr 7 accepts a photon to populate the excited state (Acr 7*) as it has a strong absorption cross section in the green light region of the visible light spectrum. The high energy state intermediate Acr 7* being both a strong reductant [E_{1/2}(C^{⋅++}/C⁺*)=-1.85 V vs SCE in MeCN] and a strong oxidant [E_{1/2}(C⁺*/C^{⋅})=+1.15 V vs SCE in MeCN], can undergo oxidative and reductive quenching cycles. During oxidative quenching cycle (FIG. 7 top), single-electron transfer with the aryl halide 67 [(E_{1/2}^{red})≈−1.85 V vs SCE in MeCN] will result in aryl radical 68 and the oxidized acridinium photocatalyst Acr 7^{+⋅}. Then, electron deficient Acr 7^{+⋅} radical dication [(E_{1/2}^{red})=+0.58 V vs SCE in MeCN] can readily accept an electron from the electron-rich enamine 69 [(E_{1/2}^{ox})≤+0.58 V vs SCE in MeCN] to generate the enaminyl/iminium radical cation 70 and thereby completing the photoredox cycle. We then hypothesized that the aryl radical species 68 can forge the new C(sp³)—C(sp²) bond by radical-radical coupling with iminium radical cation 70 or initiate radical chain propagation with the enamine 69. In case of reductive quenching cycle, the excited state photocatalyst Acr 7* accepts an electron from a donor moiety to form Acr 7^{⋅} radical (FIG. 7, bottom). We assume that the donor molecule can be pyrrolidine 12 or the in situ formed enamine 69. According to the previous reports^{21,59}, the alkylamine radical cation (D^{+⋅}) can generate an aryl radical 68 from the corresponding aryl halide 67 via halogen atom transfer (XAT), which can initiate radical chain propagation in presence of an enamine 69. In both cases, upon hydrolysis of the iminium intermediate 71, we can expect the regeneration of the ketone functional group via the removal of the amine to furnish the final desired product 72.

Conventional methods to synthesize isocoumarin molecules include using a transition metal catalyst in the presence of carbon monoxide as follows:

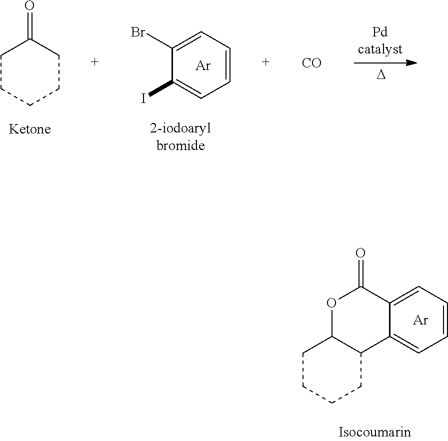

In contrast, the methods of the present invention do not require carbon monoxide and an elevated temperature higher than 50° C. The preparation of isocoumarin by a method of the present invention is schematically illustrated below.

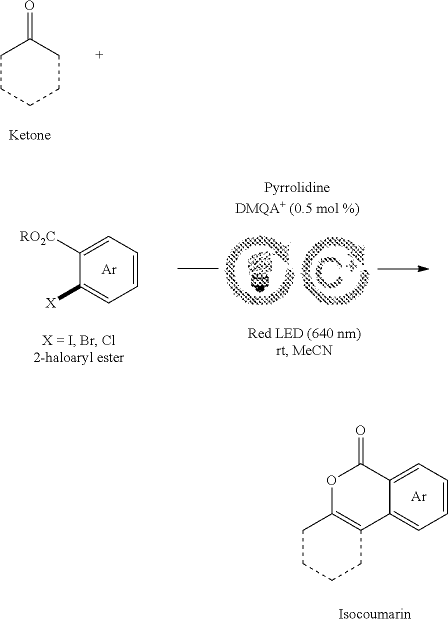

The methods of the present invention are broad in scope with unactivated ketones and tolerant of variety of functional groups. The methods are proven to be significantly powerful and clean on a multi-gram scale reaction. Importantly, its utility was exemplified in the synthesis of several economically important building blocks for diverse array of bioactive and pharmaceutical agents. Given the versatility and operational simplicity of this approach, it is expected that methods of the invention will provide a new but clean reaction platform to attain a sustainable and practical synthetic field.

### EXAMPLES

### Synthesis of the New Acridinium Series

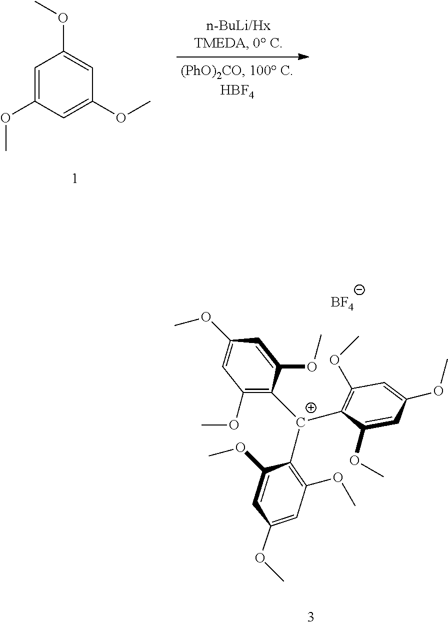

Compound 3 was synthesized according to a procedure modified from that reported by Laursen et al., J. Am. Chem. Soc., 120, 12255-12263, 1998.

An oven dried (180° C.) 500 mL three-necked round bottom flask equipped with two oven dried addition funnels and a reflux condenser was charged with a magnetic stir bar and 1,3,5-trimethoxy benzene (13.45 g, 80 mmol, 1.0 equiv). The complete setup was vacuumed for 5 mins, then purged with nitrogen and the process was repeated three times to ensure inert condition. Under nitrogen atmosphere, freshly distilled N,N,N′,N′-tetramethylethylenediamine (TMEDA) (3.6 mL, 24 mmol, 0.3 equiv) and dry toluene (50 mL) were introduced using an addition funnel. The reaction mixture was cooled to 0° C. and “BuLi (50 mL, 1.6 M in hexanes, 80 mmol, 1.0 equiv) was introduced in the other addition funnel via a cannula.” BuLi solution was added dropwise to the reaction mixture from the addition funnel at 0° C. After the completion of the addition, the reaction mixture was allowed to reach room temperature and stirred for 1 h.

In an oven dried round bottom flask, under nitrogen atmosphere, diphenyl carbonate (5.14 g, 24 mmol, 0.3 equiv) was dissolved in 40 mL toluene (sonication was required). Then this solution was quickly added to the reaction mixture and the reaction mixture was stirred at 100° C. overnight. After 18 h, the reaction mixture was allowed to cool to room temperature and the solvent was removed using a rotovap. The solid residue was dissolved in 250 mL DCM and washed with three portions of 100 ml water. After washing with 100 mL saturated aq NaHCO₃ solution, the organic layer was dried over Na₂SO₄ and concentrated to dryness under vacuum. The residue was then dissolved in 25 mL MeOH and 5 mL HBF₄ solution (47% w/w in water) was added while stirring. Then this solution was added dropwise to 500 mL Et₂O and stirred for 30 mins. A green precipitate was filtered out and washed several times with excess Et₂O. After overnight drying under vacuum, 12.36 g green solid was collected as desired product (20.6 mmol, 86% yield).

¹H NMR (500 MHz, CDCl₃) δ 6.05 (s, 6H), 3.98 (s, 9H), 3.58 (s, 18H). ¹³C NMR (126 MHz, CDCl₃) δ 170.00, 164.21, 118.86, 91.77, 56.78, 56.64. ¹⁹F NMR (470 MHz, CDCl₃) δ-153.63, -153.68.

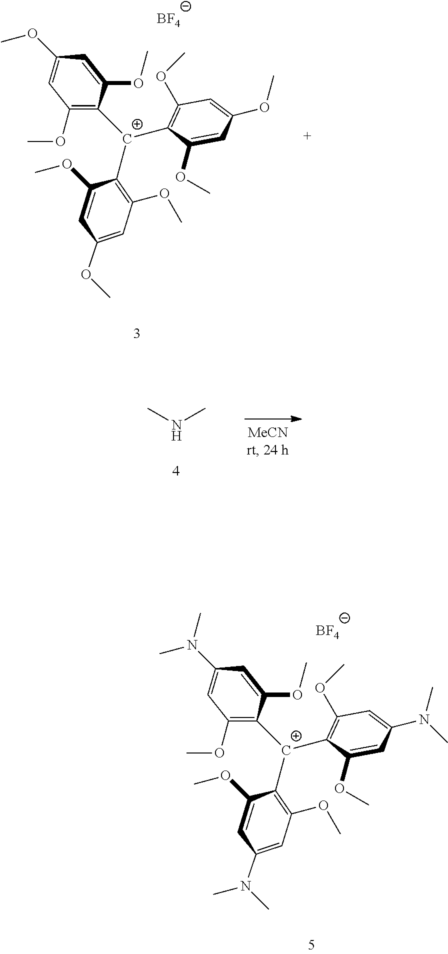

Tris(4-(dimethylamino)-2,6-dimethoxyphenyl)methylium tetrafluoroborate (5): To a stirred solution of compound 3 (3.0 g, 5.0 mmol, 1.0 equiv) in 50 mL MeCN was added dimethylamine (4) (4.51 g, 0.1 mol, 20.0 eqiv.) dropwise. Immediately it formed a red solution. After being stirred for 24 hours at room temperature, the solution's red color changed to dark blue. Then the reaction mixture was added dropwise to 500 mL Et₂O to precipitate the crude product 5 as a dark blue-green solid. Then the solid product was separated through filtration and washed 3 times with Et₂O. After layering in DCM/Hexanes, dark blue crystals were formed which were collected as product 5 (2.88 g, 90% overall yield).

¹H NMR (500 MHz, CDCl₃) δ 5.81 (s, 6H), 3.48 (s, 18H), 3.12 (s, 18H). ¹³C NMR (126 MHz, CD 3 CN) δ 164.01, 157.20, 116.49, 90.06, 56.62, 40.75. ¹⁹F NMR (470 MHz, CD 3 CN) δ-151.79, -151.84. HRMS (ESI) exact mass calculated for (C₃₁H₄₂N₃O₆⁺) requires m/z 552.3068, found m/z 552.3071.

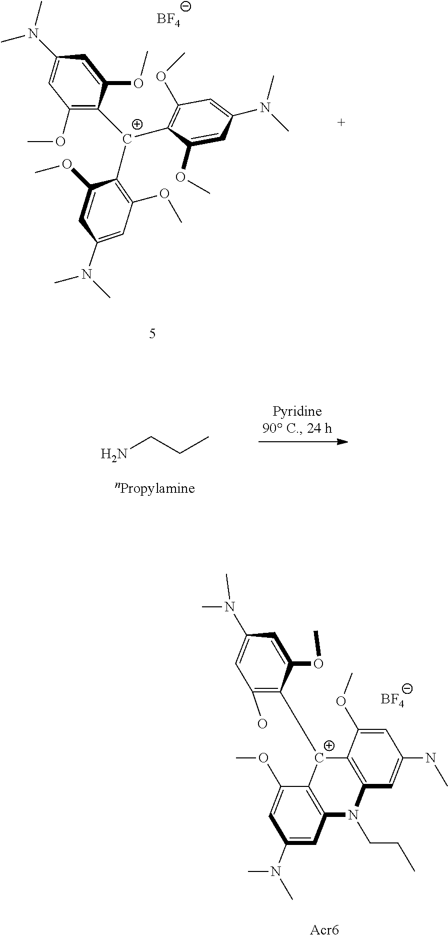

3,6-bis(dimethylamino)-9-(4-(dimethylamino)-2,6-dimethoxyphenyl)-1,8-dimethoxy-10-propyl-9,10-dihydroacridin-9-ylium tetrafluoroborate (Acr 6): A 100 ml pressure vessel equipped with a magnetic stir bar was charged with 0.64 g compound 5 (1.0 mmol, 1.0 equiv) and 0.82 mL ⁿpropylamine (10.0 mmol, 10.0 equiv) in 10 mL pyridine. Then the vessel was tightly closed with a stopper and the solution was stirred for 24 h at 90° C. The color of the reaction mixture changed from dark blue to orange-brown indicating the full consumption of compound 5. Then the reaction mixture was allowed to cool to room temperature and added dropwise to excess Et₂O (500 ml) while stirring. Immediately, an orange precipitate was observed and filtered out using gravity filtration. The orange solid was then recrystallized using slow evaporation in CH₂Cl₂/hexanes or MeCN/Et₂O. The title compound was obtained as fine crystals of green-orange color (577 mg, 91% overall yield).

¹H NMR (400 MHz, CD 3 CN) δ 6.24 (d, J=2.2 Hz, 2H), 6.22 (d, J=2.3 Hz, 2H), 6.06 (s, 2H), 4.43 (m, 2H), 3.55 (s, 6H), 3.47 (s, 6H), 3.22 (s, 12H), 3.00 (s, 6H), 2.09 (m, 2H), 1.21 (t, J=7.4 Hz, 3H). ¹³C NMR (101 MHz, CD₃CN) δ 162.85, 157.48, 156.59, 153.10, 150.33, 144.46, 111.82, 110.45, 94.47, 90.24, 88.02, 57.30, 56.20, 52.36, 41.18, 40.69, 19.65, 11.51. ¹⁹F NMR (376 MHz, CD3CN) δ-151.82, -151.88.

HRMS (ESI) exact mass calculated for (C₃₂H₄₄N₄O₄⁺) requires m/z 547.3279, found m/z 547.3281.

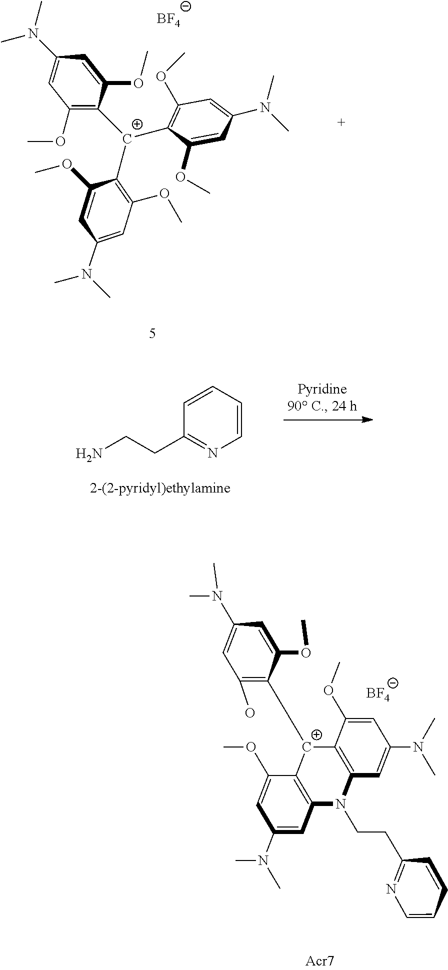

3,6-bis(dimethylamino)-9-(4-(dimethylamino)-2,6-dimethoxyphenyl)-1,8-dimethoxy-10-(2-(pyridine-2-yl)ethyl)-9,10-dihydroacridin-9-ylium tetrafluoroborate (Acr 7): A 100 ml pressure vessel equipped with a magnetic stir bar was charged with 1.28 g compound 5 (2.0 mmol, 1.0 equiv) and 2.4 mL 2-(2-pyridyl)ethylamine (20.0 mmol, 10.0 equiv) in 20 mL pyridine. Then the vessel was tightly closed with a stopper and the solution was stirred for 24 h at 90° C. The color of the reaction mixture changed from dark blue to orange-brown indicating the full consumption of compound 5. Then the reaction mixture was allowed to cool to room temperature and added dropwise to excess Et₂O (500 ml) while stirring. Immediately, an orange precipitate was observed and filtered out using gravity filtration. The orange solid was then recrystallized using slow evaporation in CH₂Cl₂/hexanes or MeCN/Et₂O. The title compound was obtained as fine crystals of green-orange color (1.22 g, 88% overall yield).

1H NMR (400 MHz, CD 3 CN) δ 8.61 (d, J=3.5 Hz, 1H), 7.74 (tt, J=7.6, 1.6 Hz, 1H), 7.38 (d, J=7.7 Hz, 1H), 7.28 (dd, J=7.5, 4.9 Hz, 1H), 6.61 (d, J=2.2 Hz, 2H), 6.23 (d, J=2.1 Hz, 2H), 6.07 (s, 2H), 4.90 (t, J=8.0 Hz, 2H), 3.56 (s, 6H), 3.48 (s, 6H), 3.44 (p, J=7.9 Hz, 2H), 3.24 (s, 12H), 3.01 (s, 6H). ¹³C NMR (126 MHz, CD 3 CN) δ 162.91, 158.80, 156.76, 153.09, 150.68, 150.40, 144.52, 137.93, 124.74, 123.17, 111.79, 110.53, 94.45, 90.29, 88.39, 57.29, 56.23, 50.56, 41.21, 34.44. ¹⁹F NMR (376 MHz, CD 3 CN) δ-151.77, -151.82.

HRMS (ESI) exact mass calculated for (C₃₆H₄₅N₅O₄⁺) requires m/z 610.3388, found m/z 610.3386.

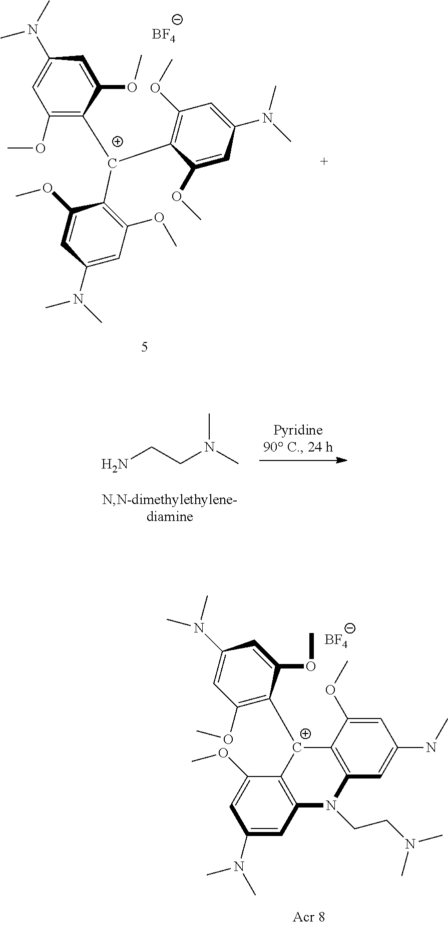

3,6-bis(dimethylamino)-9-(4-(dimethylamino)-2,6-dimethoxyphenyl)-10-(2-(dimethylamino) ethyl)-1,8-dimethoxy-9,10-dihydroacridin-9-ylium tetrafluoroborate (Acr 8) : A 100 ml pressure vessel equipped with a magnetic stir bar was charged with 0.64 g compound 5 (1.0 mmol, 1.0 equiv) and 1.1 mL N,N-dimethylethylenediamine (10.0 mmol, 10.0 equiv) in 10 mL pyridine. Then the vessel was tightly closed with a stopper and the solution was stirred for 24 h at 90° C. The color of the reaction mixture changed from dark blue to orange-brown indicating the full consumption of compound 5. Then the reaction mixture was allowed to cool to room temperature and added dropwise to excess Et₂O (500 ml) while stirring. Immediately, an orange precipitate was observed and filtered out using gravity filtration. The orange solid was then recrystallized using slow evaporation in CH₂Cl₂/hexanes or MeCN/Et₂O. The title compound was obtained as fine crystals of green-orange color (597 mg, 90% overall yield).

¹H NMR (500 MHz, CD₃CN) δ 6.43 (d, J=2.2 Hz, 2H), 6.23 (d, J=2.1 Hz, 2H), 6.07 (s, 2H), 4.57 (t, J=6.8 Hz, 2H), 3.55 (s, 6H), 3.48 (s, 6H), 3.22 (s, 12H), 3.01 (s, 6H), 2.93 (t, J=6.8 Hz, 2H), 2.40 (s, 6H). ¹³C NMR (126 MHz, CD₃CN) δ 162.86, 157.50, 156.62, 153.13, 150.51, 144.85, 111.85, 110.40, 94.43, 90.26, 88.69, 57.31, 56.21, 50.97, 46.08, 41.19, 40.70. ¹⁹F NMR (376 MHz, CD₃CN) δ-151.77, -151.82.

HRMS (ESI) exact mass calculated for (C₃₃H₄₇N₅O₄⁺) requires m/z 576.3544, found m/z 576.3547.

### Photophysical and Electrochemical Properties of Acridiniums

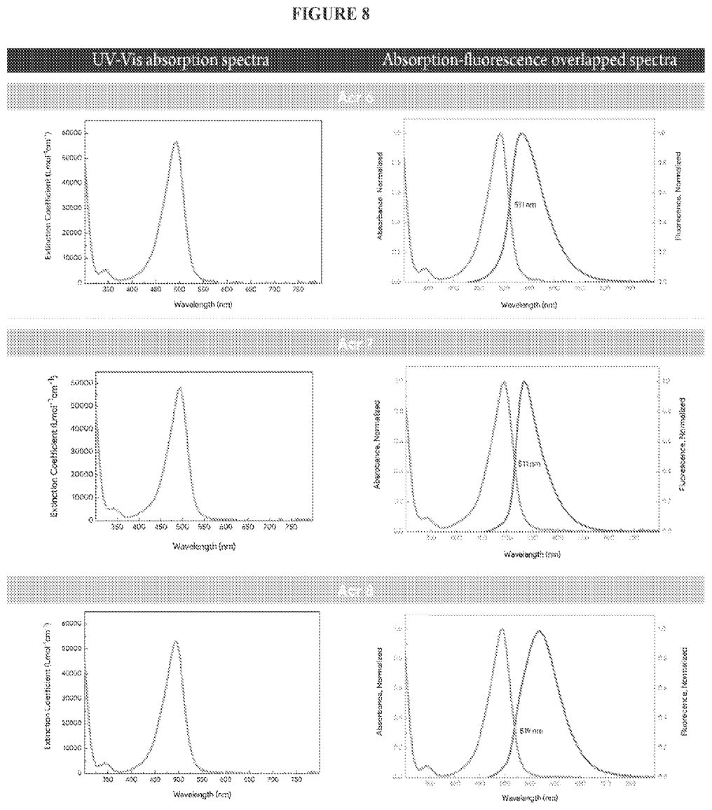

The photophysical properties of all the synthesized acridines were recorded in MeCN. The UV-Vis absorption spectra for all these acridines feature same peak absorption at 494 nm, albeit the molar extinction coefficients were different. Acridine 7 demonstrated the highest molar extinction coefficient of 58066 Lmol⁻¹ cm−1 where the lowest molar extinction coefficient was observed in Acridine 8. However, the peak emissions were observed at 534 nm, 538 nm and 568 nm for Acridine 6, 7 and 8 respectively. In addition, the normalized absorption and emission spectra are overlapped so as to find the intersection wavelengths (λ_{intersection}). See FIG. 8.

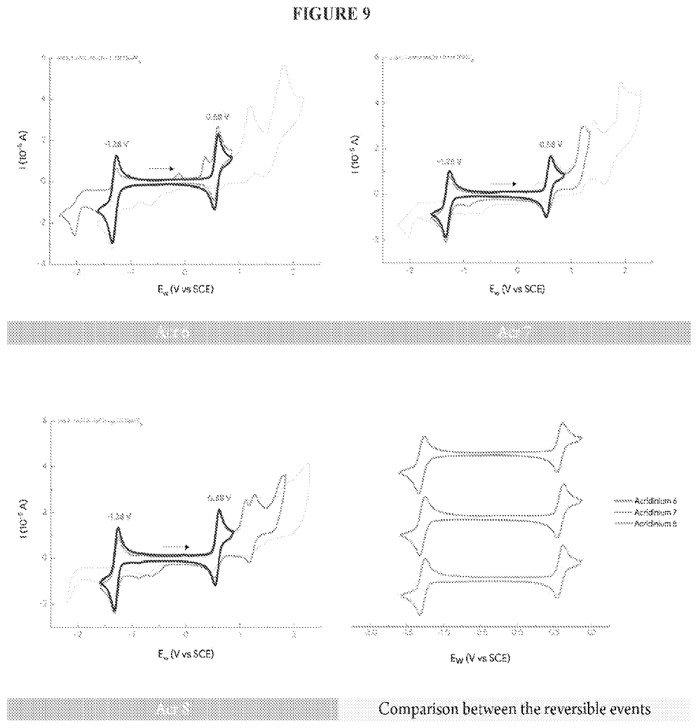

The electrochemical behavior of 6, 7 and 8 were recorded in MeCN by cyclic voltammetry (CV). All of these compounds demonstrated identical oxidation and reduction events in the cyclic voltammogram. All the cyclic voltammogram featured a fully reversible oxidation of C⁺ to C^{⋅++} (E_{1/2}(C^{⋅++}/C⁺)) at +0.58 V versus the saturated calomel electrode (SCE) and a fully reversible reduction of C⁺ to C^{⋅} (E_{1/2}(C⁺/C^{⋅})) at −1.28 V versus SCE. See FIG. 9.

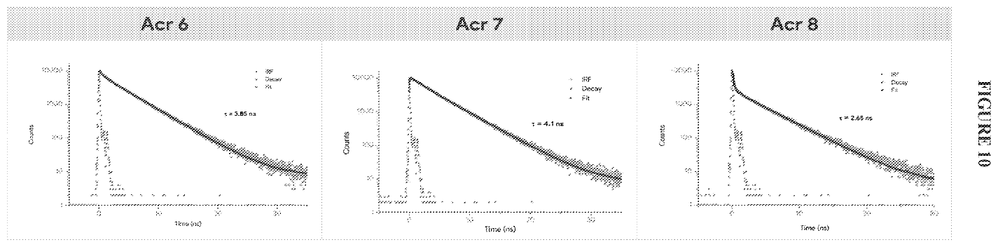

FIG. 10 shows the lifetime spectra for acridiniums 6, 7 and 8.

Based on the reported literature (Romero et al., Chem. Rev., 116, 10075-10166, 2016) the excitation energy (E_{0,0}) is estimated by calculating the energy of the wavelength at which the compound's normalized UV-Vis absorption and emission spectra overlap. The excited state reduction potential E_{1/2}(C^{+⋅}/C^{⋅}) is calculated by E_{1/2}(C⁺⋅/C⋅)=E_{1/2}(C^{+⋅}/C^{⋅})+E_{0,0}; while the excited state oxidation potential E_{1/2} (C⁺⁺/C⁺*) is calculated by E_{1/2}(C^{⋅++}/C^{+⋅})=E_{1/2}(C^{⋅++}/C^{+⋅})−E_{0,0} . Thus, we can obtain the corresponding photophysical and electrochemical properties of acridine 6, 7 and 8. As an example, the detailed calculation by using the values of acridine 7 in MeCN is given below.
Absorption λₘₐₓ=494 nm Emission λₘₐₓ=538 nm
Intersection wavelength λ_{intersection}=511 nm
E_{0/0}=hc/λ_{intersection}=1240.78476/511 eV=2.43 eV
E_{1/2}(C^{⋅++}/C^{+⋅})=0.58 V−2.43 V=−1.85 V
E_{1/2}(C^{+⋅}/C^{⋅})=−1.28 V+2.43 V=+1.15 V.

The table below provides a summary of the photophysical and electrochemical properties of acridiniums 6, 7 and 8.

| | | | Intersection | | | | | | Excited |
|---|---|---|---|---|---|---|---|---|---|
| | Absorption | Emmission | wavelength | | | | | | state |
| | [symbol] | [symbol] | [symbol] | [symbol] | [symbol] (C [symbol] /C [symbol] ) | [symbol] (C [symbol] /C [symbol] ) | [symbol] (C [symbol] /C [symbol] ) | [symbol] (C [symbol] /C [symbol] ) | lifetime [symbol] |
| Acridine | (nm) | (nm) | (nm) | (eV) | (V) | (V) | (V) | (V) | (ns) |
| 6 | 494 | 534 | 511 | 2.43 | +0.58 | −1.28 | −1.85 | +1.15 | 2.85 |
| 7 | 494 | 538 | 511 | 2.43 | +0.58 | −1.28 | −1.85 | +1.15 | 4.10 |
| 8 | 494 | 568 | 519 | 2.39 | +0.58 | −1.28 | −1.81 | +1.11 | 2.65 |

[symbol]

indicates data missing or illegible when filed

### Reaction Optimization of Photoredox α-Arylation of Ketones

### Screening of New Acridiniums

Inside a N₂ glovebox, an oven-dried 10 mL schlenk flask equipped with a magnetic stir bar was charged with 4-iodobenzonitrile (22.9 mg, 0.1 mmol, 1.0 equiv), cyclohexanone (52 μL, 0.5 mmol, 5.0 equiv), pyrrolidine (42 μL, 0.5 mmol, 5.0 equiv), corresponding photocatalyst (Acr 6-8, x mol %), and dry MeCN (1 mL, 0.1 M). Then the Schlenk flask was placed in a water bath approximately 5 cm away from a Kessil 518 nm Green LED lamp. After 24 h, the reaction mixture was evaporated under vacuum. Then 16.8 mg of 1,3,5-trimethoxybenzene (0.1 mmol, 1 equiv.) was added as internal standard and dissolved in 0.7 mL CDQ₃. All the yields are calculated from ¹H NMR spectra using 1,3,5-trimethoxy benzene as internal standard.

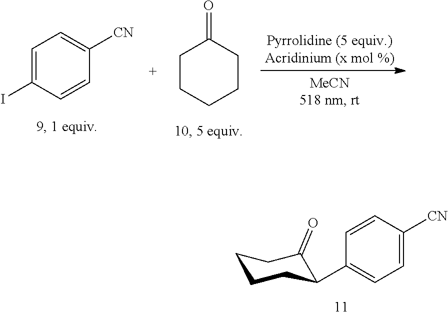

| | | Equivalent | | Dehalogenated | α-CO arylated |
|---|---|---|---|---|---|
| Entry | Photocatalyst | amount | SM | product | Product 11 |
| 2a | Acr 6 | 5.0 mol % | trace | 17% | 72% |
| 2b | | 0.2 mol % | trace | 15% | 75% |
| 2c | Acr 7 | 5.0 mol % | trace | 16% | 71% |
| 2d | | 2.0 mol % | trace | 15% | 72% |
| 2e | | 1.0 mol % | trace | 15% | 70% |
| 2f | | 0.5 mol % | trace | 13% | 75% |
| 2g | | 0.2 mol % | trace | 14% | 75% |
| 2h | | 0.1 mol % | 5% | 13% | 66% |
| 2i | Acr 8 | 5.0 mol % | trace | 16% | 70% |
| 2j | | 0.2 mol % | trace | 16% | 73% |

### Screening of Amines

Inside a N₂ glovebox, an oven-dried 10 mL schlenk flask equipped with a magnetic stir bar was charged with 4-iodobenzonitrile (22.9 mg, 0.1 mmol, 1.0 equiv), cyclohexanone (52 μL, 0.5 mmol, 5.0 equiv), amine (5.0 equiv), Acr 7 (0.14 mg, 0.0002 mmol, 0.002 equiv., 0.2 mol %), and dry MeCN (1 mL, 0.1 M). Then the Schlenk flask was placed in a water bath approximately 5 cm away from a Kessil 518 nm Green LED lamp. After 24 h, the reaction mixture was evaporated under vacuum. Then 16.8 mg of 1,3,5-trimethoxybenzene (0.1 mmol, 1 equiv.) was added as internal standard and dissolved in 0.7 mL CDCl₃. All the yields are calculated from ¹H NMR spectra using 1,3,5-trimethoxy benzene as internal standard.

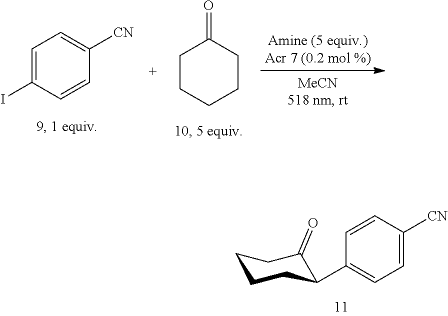

| | | | | Dehalogenated | α-CO arylated |
|---|---|---|---|---|---|
| Entry | Acr 7 | Amine | SM | product | Product 11 |
| 3a | 0.2 mol % | Azepane | 8% | 25% | 32% |
| 3b | | Piperidine | 6% | 60% | 12% |
| 3c | | Pyrrolidine | trace | 14% | 75% |
| 3d | | iPrBnNH | 90% | trace | 3% |
| 3e | | Cy₂NH | 88% | 6% | trace |
| 3f | | Me₂NH | 75% | 15% | 2% |
| 3g | | Et₂NH | 36% | 44% | 6% |
| 3h | | iPr₂NH | 22% | 67% | 4% |
| 3i | | (4-t-butylphtnyl)₂NH | 82% | trace | trace |

### Screening of Ketone and Amine Equivalence

Inside a N₂ glovebox, an oven-dried 10 mL schlenk flask equipped with a magnetic stir bar was charged with 4-iodobenzonitrile (22.9 mg, 0.1 mmol, 1.0 equiv), cyclohexanone (x equiv), amine (y equiv), Acr 7 (0.14 mg, 0.0002 mmol, 0.002 equiv., 0.2 mol %), and dry MeCN (1 mL, 0.1 M). Then the Schlenk flask was placed in a water bath approximately 5 cm away from a Kessil 518 nm Green LED lamp. After 24 h, the reaction mixture was evaporated under vacuum. Then 16.8 mg of 1,3,5-trimethoxybenzene (0.1 mmol, 1 equiv.) was added as internal standard and dissolved in 0.7 mL CDCl₃. All the yields are calculated from ¹H NMR spectra using 1,3,5-trimethoxy benzene as internal standard.

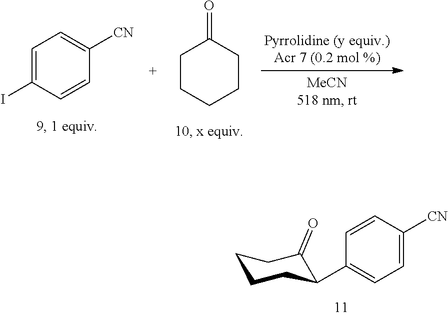

| | | | | | Dehalogenated | α-CO arylated |
|---|---|---|---|---|---|---|
| Entry | Acr 7 | Cyclohexanone | Pyrrolidine | SM | product | Product 11 |
| 4a | 1.0 mol % | 5 equiv | 5 equiv | trace | 15% | 70% |
| 4b | | 1 equiv | 5 equiv | 7% | 15% | 52% |
| 4c | | 1 equiv | 1 equiv | 7% | 7% | 38% |
| 4d | | 5 equiv | 1 equiv | 5% | 20% | 60% |
| 4e | | 3 equiv | 3 equiv | trace | 18% | 74% |
| 4f | 0.5 mol % | 5 equiv | 5 equiv | trace | 13% | 75% |
| 4g | 0.2 mol % | 5 equiv | 5 equiv | trace | 14% | 75% |
| 4h | | 3 equiv | 3 equiv | trace | 16% | 72% |
| 4i | | 2 equiv | 2 equiv | trace | 13% | 65% |
| 4j | | 1.5 equiv | 1.5 equiv | 15% | 12% | 51% |
| 4k | 0.1 mol % | 5 equiv | 5 equiv | 5% | 13% | 66% |

### Screening of Solvents

Inside a N₂ glovebox, an oven-dried 10 mL schlenk flask equipped with a magnetic stir bar was charged with 4-iodobenzonitrile (22.9 mg, 0.1 mmol, 1.0 equiv), cyclohexanone (52 μL, 0.5 mmol, 5.0 equiv), pyrrolidine (42 μL, 0.5 mmol, 5.0 equiv), Acr 7 (0.14 mg, 0.0002 mmol, 0.002 equiv., 0.2 mol %), and dry solvent (1 mL, 0.1 M). Then the Schlenk flask was placed in a water bath approximately 5 cm away from a Kessil 518 nm Green LED lamp. After 24 h, the reaction mixture was evaporated under vacuum. In case of high boiling solvents (DMSO, DMF, DMPU), 5 mL 1.0 N HCl was added to the reaction mixture and was extracted with three portions of 5 mL Et₂O. Combined organic layer was washed with brine, dried using Na₂SO₄, concentrated under vacuum. Then 16.8 mg of 1,3,5-trimethoxybenzene (0.1 mmol, 1 equiv.) was added as internal standard and dissolved in 0.7 mL CDCl₃. All the yields are calculated from ¹H NMR spectra using 1,3,5-trimethoxy benzene as internal standard.

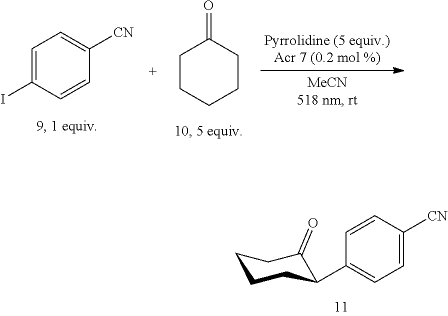

| | | | Dehalogenated | α-CO arylated |
|---|---|---|---|---|
| Entry | Amine | SM | product | Product 11 |
| 5a | DCM | 37% | 12% | 45% |
| 5b | PhCF₂ | 16% | 25% | 55% |
| 5c | MeCN | trace | 14% | 75% |
| 5d | DMSO | 15% | 18% | 58% |
| 5e | DMF | 7% | 22% | 64% |
| 5f | DMPU | 46% | 30% | 23% |

### Screening of Light Sources

Inside a N₂ glovebox, an oven-dried 10 mL schlenk flask equipped with a magnetic stir bar was charged with 4-iodobenzonitrile (22.9 mg, 0.1 mmol, 1.0 equiv), cyclohexanone (52 μL, 0.5 mmol, 5.0 equiv), pyrrolidine (42 μL, 0.5 mmol, 5.0 equiv), Acr 7 (0.14 mg, 0.0002 mmol, 0.002 equiv., 0.2 mol %), and dry MeCN (1 mL, 0.1 M). Then the Schlenk flask was placed in a water bath approximately 5 cm away from a light source. After 24 h, the reaction mixture was evaporated under vacuum. Then 16.8 mg of 1,3,5-trimethoxybenzene (0.1 mmol, 1 equiv.) was added as internal standard and dissolved in 0.7 mL CDCl₃. All the yields are calculated from ¹H NMR spectra using 1,3,5-trimethoxy benzene as internal standard.

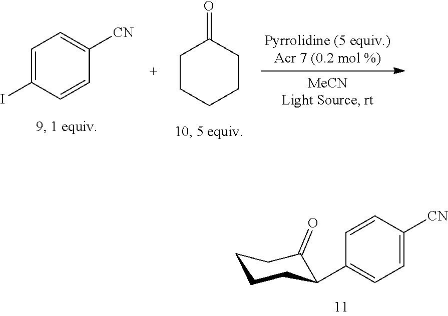

| | | | Dehalogenated | α-CO arylated |
|---|---|---|---|---|
| Entry | Light Source | SM | product | Product 11 |
| 6a | 467 nm | trace | 18% | 70% |
| 6b | 518 nm | trace | 14% | 75% |
| 6c | 23 W White LED | trace | 35% | 58% |
| 6d | No light | 98% | trace | trace |

### Mechanistic Investigations

### Control Experiments

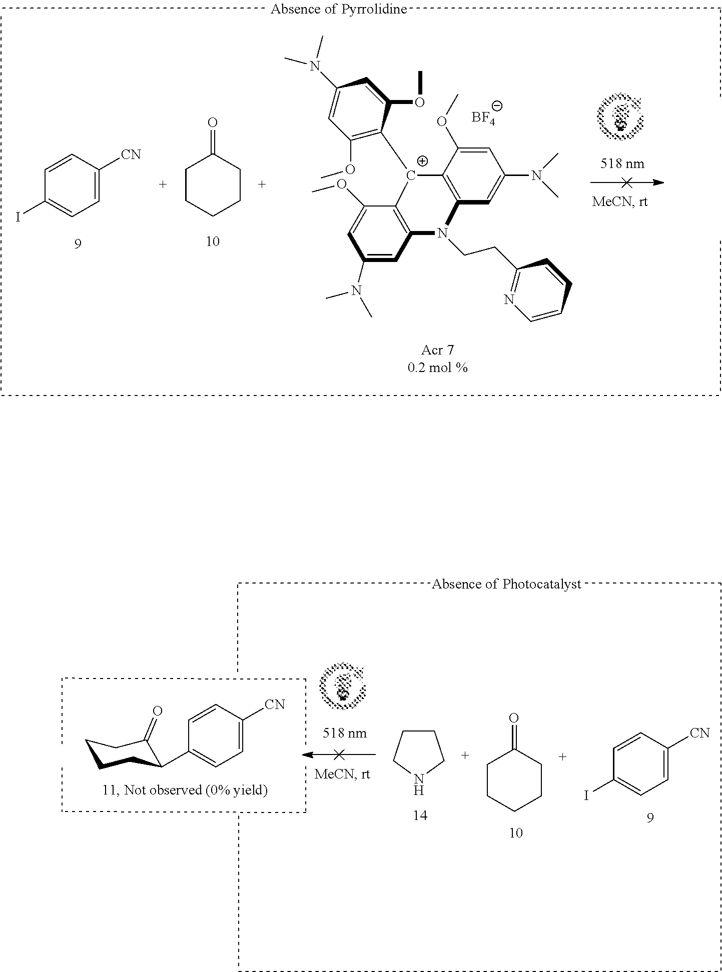

### Control Reactions in the Absence of Amine and Photocatalyst

Without Pyrrolidine: Inside a N₂ glovebox, an oven-dried 10 mL schlenk flask equipped with a magnetic stir bar was charged with 4-iodobenzonitrile (22.9 mg, 0.1 mmol, 1.0 equiv), cyclohexanone (52 μL, 0.5 mmol, 5.0 equiv), Acr 7 (0.14 mg, 0.0002 mmol, 0.002 equiv., 0.2 mol %) and dry MeCN (1 mL, 0.1 M). Then the Schlenk flask was placed in a water bath approximately 5 cm away from a Kessil 518 nm Green LED lamp. After 24 h, the reaction mixture was evaporated under vacuum. Then 16.8 mg of 1,3,5-trimethoxybenzene (0.1 mmol, 1 equiv.) was added as internal standard and dissolved in 0.7 mL CDCl₃. No desired product 11 was observed in the ¹H NMR while the starting material remained unconsumed.

Without Photocatalyst: Inside a N₂ glovebox, an oven-dried 10 mL schlenk flask equipped with a magnetic stir bar was charged with 4-iodobenzonitrile (22.9 mg, 0.1 mmol, 1.0 equiv), cyclohexanone (52 μL, 0.5 mmol, 5.0 equiv), pyrrolidine (42 μL, 0.5 mmol, 5.0 equiv) and dry MeCN (1 mL, 0.1 M). Then the Schlenk flask was placed in a water bath approximately 5 cm away from a Kessil 518 nm Green LED lamp. After 24 h, the reaction mixture was evaporated under vacuum. Then 16.8 mg of 1,3,5-trimethoxybenzene (0.1 mmol, 1 equiv.) was added as internal standard and dissolved in 0.7 mL CDCl₃. No desired product 11 was observed in the ¹H NMR while the starting material remained unconsumed.

### Alternating Light/Dark Experiment

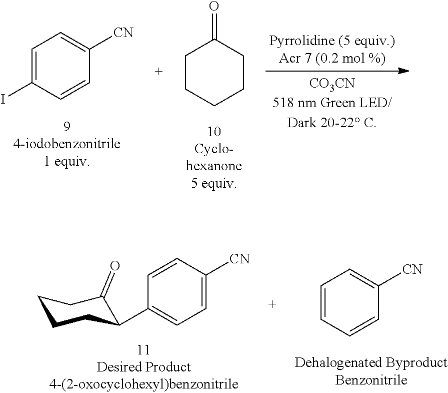

According to the literature, an amine can be involved in the halogen atom transfer from aryl halide. This can then initiate a chain propagation pathway as an alternative to the photocatalytic cycle. To investigate the possibility of such light independent mechanistic pathway, we set out to evaluate our reaction in a consecutive illuminated-dark condition in some fixed time intervals.

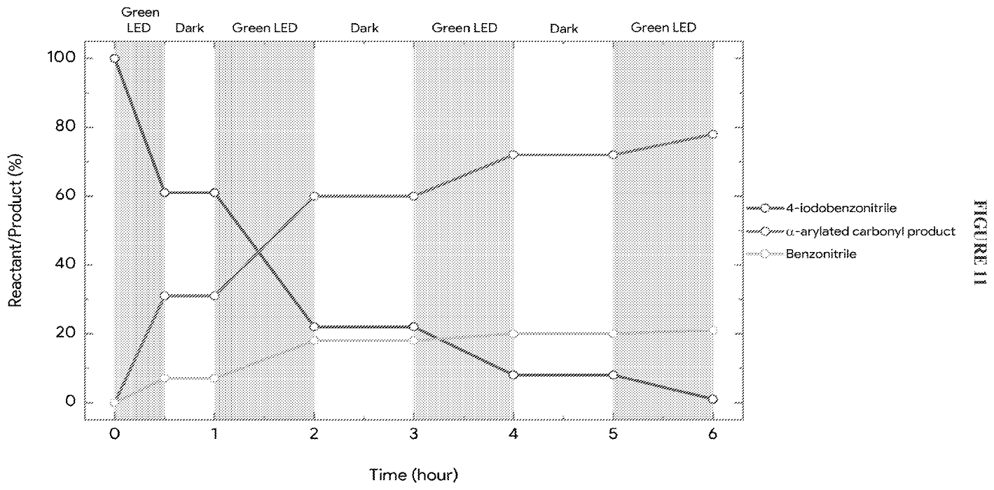

Inside a N₂ glovebox, an oven-dried an oven-dried 1.0 dram glass vial equipped with a magnetic stir bar was charged with 4-iodobenzonitrile (22.9 mg, 0.1 mmol, 1.0 equiv), cyclohexanone (52 μL, 0.5 mmol, 5. equiv), Acr 7 (0.14 mg, 0.0002 mmol, 0.002 equiv., 0.2 mol %), pyrrolidine (42 μL, 0.5 mmol, 5.0 equiv), 1,3,5-trimethoxybenzene (16.8 mg, 0.1 mmol, 1 equiv., internal standard) and dry CD 3 CN (1 mL, 0.1 M). Then the reaction mixture was stirred for a minute and then transferred to a valved NMR tube. Tightly sealed NMR tube was then placed in a water bath approximately 5 cm away from a Kessil 518 nm Green LED lamp. After 30 min, a ¹H NMR spectrum was recorded, and 31% desired α-arylated ketone product was detected along with 7% dehalogenated product and 61% unreacted 4-iodobenzonitrile. Then the NMR tube was kept in the dark for 30 min and no change in amount for any of the product and starting material was observed from the corresponding ¹H NMR spectrum. Afterwards, the reaction mixture was illuminated for 1 h resulting increased α-arylated ketone product (61% NMR yield) and decreased starting material (22%). In accordance with the previous dark experiment observation no change was detected after 1 h absence of light this time also. This 1 h green light illumination and 1 h dark events were carried out 3 more times until full conversion of the starting material was observed. In each illuminated cases the increment of the desired product was observed while the starting material decreased. However, the reaction did not proceed in any case of the 3 dark events where light was absent. The overall trend of the reaction in presence and absence of light is shown in FIG. 11.

### Evaluation of Commonly Used Organic Photocatalysts

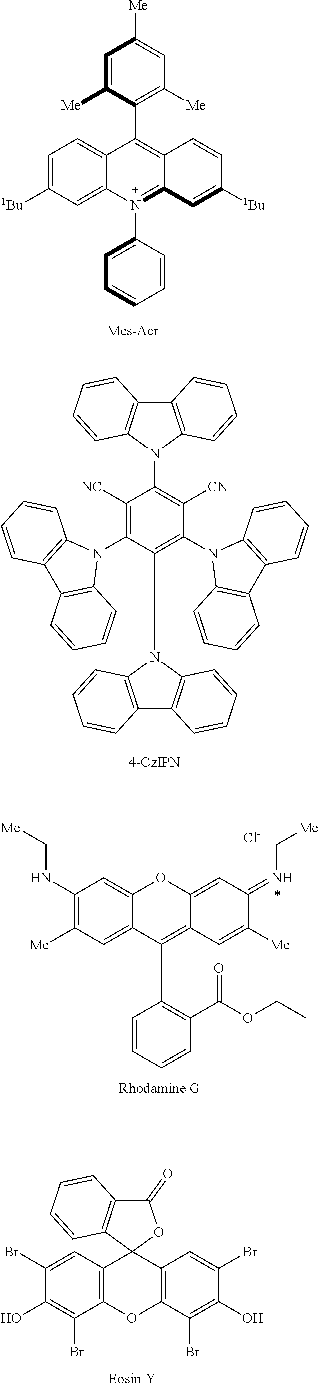

To evaluate the commonly used organic photocatalysts in this reaction methodology four widely used photocatalysts were selected for control experiments. All four catalysts were employed in the standard reaction conditions under appropriate wavelength of light sources. We have evaluated 4 different aryl iodide substrates with different reduction potential in varying reaction times. In case of electron deficient aryl iodide, we have observed similar reaction outcomes from widely used photocatalysts Mes-Acr, 4-CzIPN and Eosin Y compared to this newly introduced Acr 7. These results benchmarked the photocatalytic efficiency of this new acridinium family. In addition to that, substantial evidence for the XAT mechanistic pathway was also understood as none of these traditional photocatalysts can reach that low reduction potential (4-iodobenzonitrile, E_{1/2}^{red}=−1.81 V vs SCE)⁴, even in their photoexcited states.

Most intriguing outcomes have been observed with mild electron deficient and electron rich aryl iodides such as, 4-bromo iodobenzene, 4-iodobiphenyl and 4-iodotoluene. In case of first two substrates, reaction conversion was almost two times with the new Acr 7 than the best performing Mes-Acr among the other photocatalysts. This difference in efficiency in C(sp²)—I bond activation was as high as three times with electron rich 4-iodotoluene substrate. This is clearly evident that Acr 7 outperformed the existing photocatalysts in context of C(sp²)—I bond activation as it can initiate single electron transfer (SET) pathway along with XAT in its photoexcited state.

Inside a N₂ glovebox, an oven-dried 10 mL schlenk flask equipped with a magnetic stir bar was charged with corresponding aryl halide (0.1 mmol, 1.0 equiv), cyclohexanone (52 μL, 0.5 mmol, 5.0 equiv), pyrrolidine (42 μL, 0.5 mmol, 5.0 equiv), corresponding photocatalyst (0.0002 mmol, 0.2 mol %), and dry MeCN (1 mL, 0.1 M). Then the Schlenk flask was placed in a water bath approximately 5 cm away from a Kessil LED lamp. After mentioned time period, the reaction mixture was evaporated under vacuum. Then 16.8 mg of 1,3,5-trimethoxybenzene (0.1 mmol, 1 equiv.) was added as internal standard and dissolved in 0.7 mL CDCl₃. All the yields are calculated from ¹H NMR spectra using 1,3,5-trimethoxy benzene as internal standard.

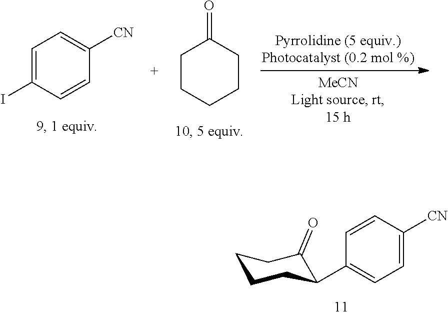

The table below shows screening of different photocatalysts with the substrate 4-iodobenzonitrile.

| | | | | Dehalogenated | α-CO arylated |
|---|---|---|---|---|---|
| Entry | Photocatalyst | Conversion | SM | product | Product 11 |
| 7a | Mes-Acr (467 nm) | 100% | — | 25% | 73% |
| 7b | Rhodamine-6G (518 nm) | 52% | 48% | 9% | 42% |
| 7c | Eosin Y (518 nm) | 100% | 2% | 24% | 72% |
| 7d | Acr 7 (518 nm) | 100% | — | 25% | 75% |

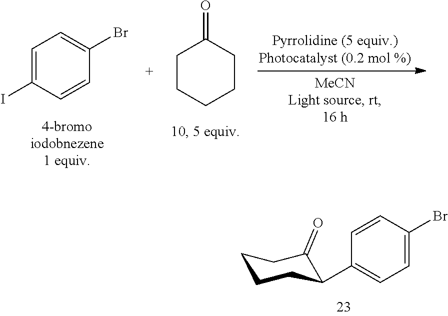

The table below shows screening of different photocatalysts with the substrate 4-bromo iodobenzene.

| | | | | Dehalogenated | α-CO arylated |
|---|---|---|---|---|---|
| Entry | Photocatalyst | Conversion | SM | product | Product 23 |
| 8a | Mes-Acr (467 nm) | 53% | 47% | 16% | 37% |
| 8b | 4-CzIPN(467 mm) | 44% | 56% | 13% | 31% |
| 8c | Rhodamine-6G (518 nm) | 18% | 82% | 6% | 12% |
| 8d | Eosin Y (518 nm) | 38% | 62% | 18% | 20% |
| 8e | Acr 7 (518 nm) | 94% | 6% | 26% | 68% |

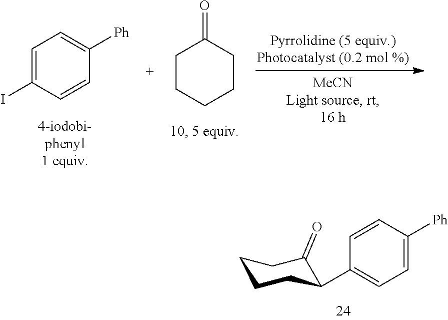

The table below shows screening of different photocatalysts with the substrate 4-iodobiphenyl.

| | | | | Dehalogenated | α-CO arylated |
|---|---|---|---|---|---|
| Entry | Photocatalyst | Conversion | SM | product | Product 24 |
| 9a | Mes-Acr (467 nm) | 38% | 62% | 13% | 25% |
| 9b | 4-CzIPN(467 mm) | 55% | 45% | 15% | 40% |
| 9c | Rhodamine-6G (518 nm) | 12% | 88% | 6% | 6% |
| 9d | Eosin Y (518 nm) | 24% | 74% | 6% | 18% |
| 9e | Acr 7 (518 nm) | 82% | 18% | 24% | 58% |

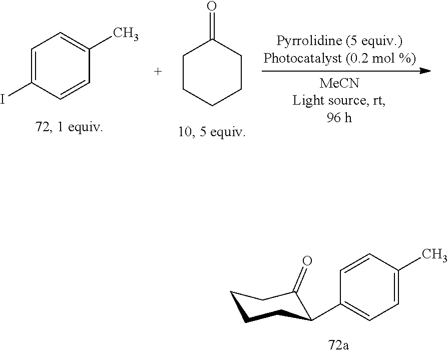

The table below shows screening of different photocatalysts with the substrate 4-iodotoluene.

| | | | | Dehalogenated | α-CO arylated |
|---|---|---|---|---|---|
| Entry | Photocatalyst | Conversion | SM | product | Product 11 |
| 10a | Mes-Acr (467 nm) | 19% | 81% | 9% | 10% |
| 10b | Acr 7 (518 nm) | 60% | 40% | 28% | 32% |

### Investigation for Aryl Radical Generation (Photo-Arbuzov Reaction)

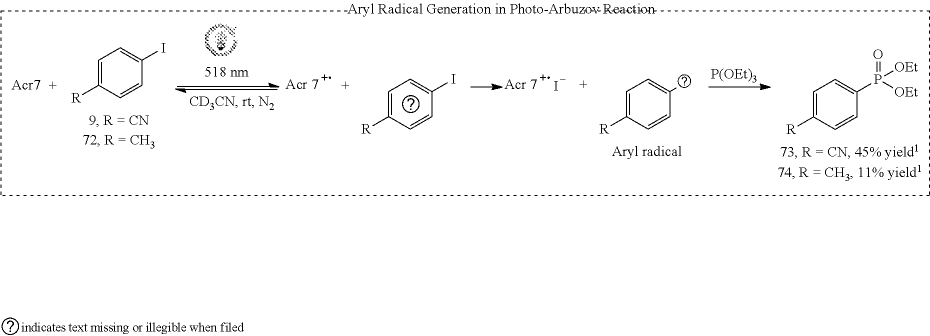

Inside a N₂ glovebox, an oven-dried 1.0 dram glass vial equipped with a magnetic stir bar was charged with Acr 7 (17.5 mg, 0.025 mmol, 1.0 equiv.), 4-iodobenzonitrile (115 mg, 0.5 mmol, 20.0 equiv), triethylphosphite (430 μL, 2.5 mmol, 100.0 equiv), and dry CD 3 CN (1 mL). Then the reaction mixture was stirred for a minute and then transferred to a valved NMR tube. Tightly sealed NMR tube was then placed in a water bath approximately 5 cm away from a Kessil 518 nm Green LED lamp. After 24 h, the formation of the aryl-phosphite adduct (73, 28% yield) was detected in 31 P NMR and ¹H NMR spectroscopy. Furthermore, 45% aryl-phosphite adduct was detected after 48h.

A similar experiment was performed with 4-iodotoluene 72 instead of 4-iodobenzonitrile. After 24 h irradiation in green light 5% of the tolyl-phosphite adduct 74 was detected in 31 P NMR while 11% adduct was observed after 48 h.

### Spectroelectrochemical Analysis

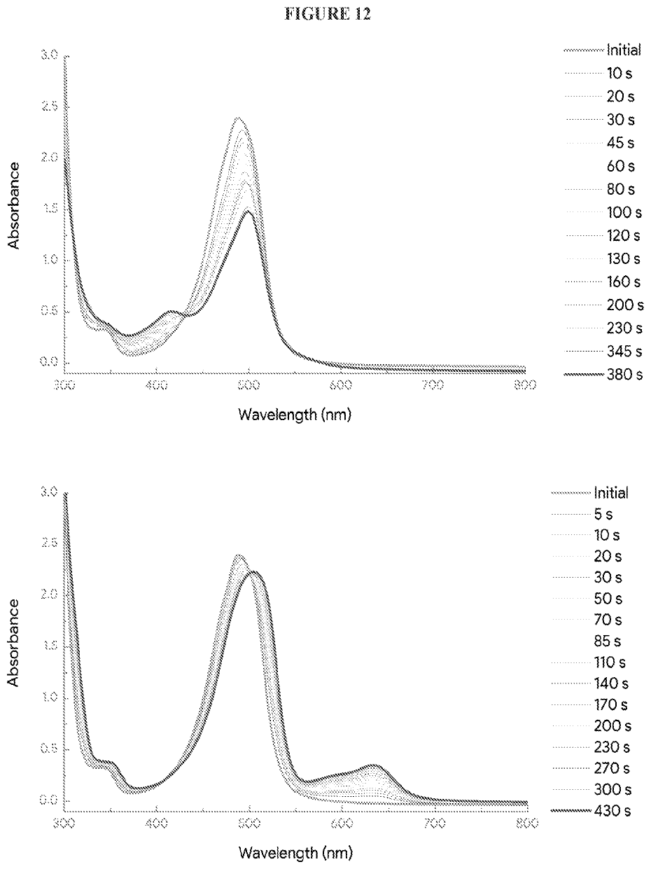

Spectroelectrochemical data for the photocatalyst Acr 7 were collected in MeCN. Appearance of neutral Acr 7 radical absorption was observed between 400 and 440 nm while the dicationic Acr 7 radical absorption appeared between 560 and 650 nm. With this information in hand, then we recorded the transient absorption spectra of Acr 7 in our standard reaction condition where 4-iodobenzonitrile and cyclohexanone were used as substrate and pyrrolidine was the amine additive. See FIG. 12.

### Transient Absorption Spectroscopy

A Ti-Sapphire laser, Libra from Coherent (800 nm source) was used for the transient absorption experiment as the source for all light generation. The pump pulse of 515 nm was generated using a commercial optical parametric amplifier (TOPAS) while the broadband white light probe pulse was generated using an Argon tube. After pump interaction, the probe pulse was routed through a delay stage to impart the required time delay before it interacted with the sample. The probe, along with signal, is then collected on a CCD to get the frequency resolution.

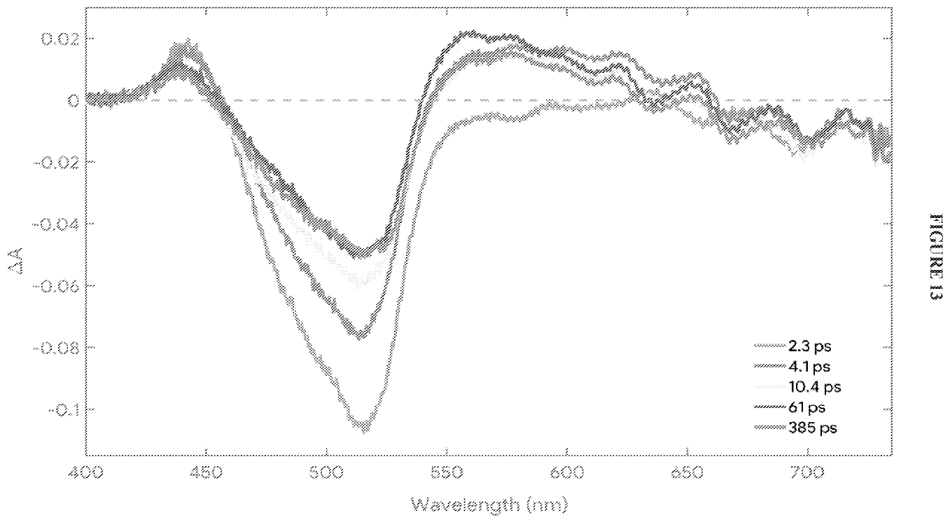

Transient absorption spectra were collected for the following reaction mixture in presence of Acr 7 in dry MeCN. See FIG. 13.

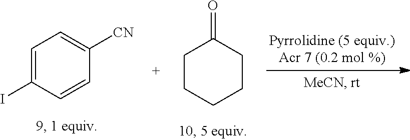 [SMILES: N#Cc1ccc(I)cc1 ; O=C1CCCCC1]

Prominent excited state absorption (ESA; ΔA>0) is apparent in the red region (dicationic acridine radical). Some ESA features for neutral acridine radical were also observed in <450 nm wavelength range. A pump pulse of 515 nm was used for this experiment which corresponds to the negative feature (ground state bleach at —515 nm) of the spectra.

### Proposed Mechanism

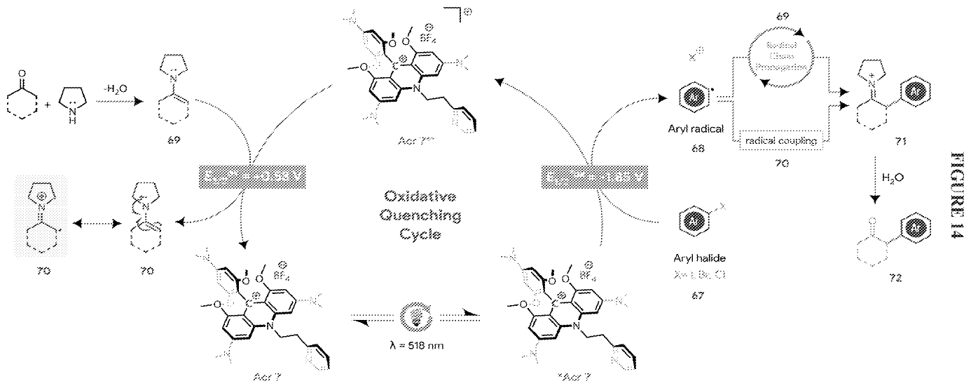

A proposed mechanism is shown in FIG. 14. First, photoexcitation of Acr 7 generates Acr 7* under green light, which reduces the aryl halide 67 to afford the aryl radical species 68 and the radical dication Acr 7^{⋅+} through a single electron transfer process (SET). At the same time, the electron-rich enamine 69 is formed in situ from corresponding ketone and pyrrolidine, which undergoes a single electron oxidation with the radical dication Acr 7^{⋅+} to form the crucial enaminyl radical cation intermediate 70 and the ground-state Acr 7, thus completing the catalytic cycle. Then, electronic rearrangement of intermediate 70 furnishes the new iminium radical intermediate 70, followed by radical-radical coupling with the aryl radical species 68 or radical chain propagation with enamine 69 to generate the iminium intermediate 71. Finally, in presence of water, hydrolysis of iminium 71 gives rise to the desired α-arylated ketone 72.

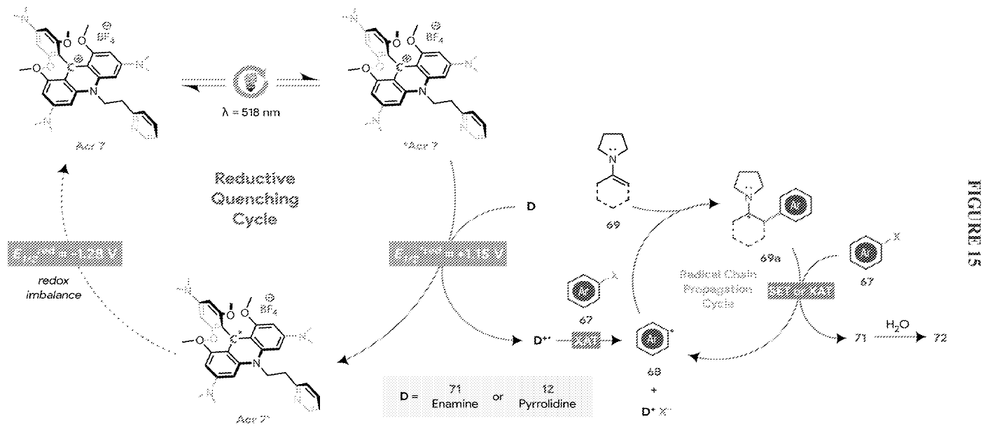

The other mechanistic pathway proposed involving reductive quenching cycle of the photocatalyst Acr 7 (FIG. 15). In this pathway, photoexcitation of Acr 7 generates Acr 7* under green light which accepts an electron from a donor moiety to form Acr 7′ radical. We assume that the donor molecule can be pyrrolidine 12, or the in situ formed enamine 69. According to the previous reports the alkylamine radical cation (D^{+⋅}) can generate an aryl radical 68 from corresponding aryl halide 67 via halogen atom transfer (XAT). This aryl radical can initiate the radical chain propagation in presence of an enamine 69 by forming an arylated amino alkyl radical 69a. This radical 69a can undergo XAT or SET with another aryl halide molecule to regenerate the aryl radical 68 continuing the radical chain propagation cycle. Meanwhile, the arylated iminium intermediate 71 is generated from 69a. Finally, in presence of water, hydrolysis of iminium 71 gives rise to the desired α-arylated ketone 72.

### Experimental Procedures

General Procedure A (GP-A): Inside a N₂ glovebox, an oven-dried 10 mL schlenk flask equipped with a magnetic stir bar was charged with Acr 7 (0.002 equiv, 0.2 mol %), corresponding aryl halide (1.0 equiv), corresponding ketone (5.0 equiv), pyrrolidine (5.0 equiv) and dry MeCN (0.1 M). Then the Schlenk flask was placed in a water bath approximately 5 cm away from a Kessil 518 nm Green LED lamp. After completion, the reaction mixture was evaporated under vacuum. To neutralize the crude reaction mixture 1.0 N HCl was added and then the aqueous layer was extracted by 3 portions of Et₂O. Combined organic layer was washed with brine, dried using Na 2 SO 4 , concentrated under vacuum and purified by flash chromatography to afford the desired product.

General Procedure B (GP-B): Inside a N₂ glovebox, an oven-dried 10 mL schlenk flask equipped with a magnetic stir bar was charged with Acr 7 (0.02 equiv, 2.0 mol %), corresponding aryl halide (1.0 equiv), corresponding ketone (5.0 equiv), pyrrolidine (5.0 equiv) and dry MeCN (0.1 M). Then the Schlenk flask was placed in a water bath approximately 5 cm away from a Kessil 518 nm Green LED lamp. After completion, the reaction mixture was evaporated under vacuum. To neutralize the crude reaction mixture 1.0 N HCl was added and then the aqueous layer was extracted by 3 portions of Et₂O. Combined organic layer was washed with brine, dried using Na₂SO₄, concentrated under vacuum and purified by flash chromatography to afford the desired product.

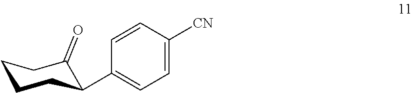

4-(2-oxocyclohexyl)benzonitrile (11): Prepared according to GP-B using Acr 7 (2.8 mg, 0.004 mmol, 0.02 equiv, 2.0 mol %), 4-bromobenzonitrile (36.4 mg, 0.2 mmol, 1.0 equiv), cyclohexanone (104 μL, 1 mmol, 5.0 equiv), pyrrolidine (84 μL, 1 mmol, 5.0 equiv) and dry MeCN (2 ml, 0.1 M). Workup was performed after 24 h and purification by flash column chromatography (30% Et₂O/Hexanes) afforded the title compound as a white solid (23 mg, 56%); mp 82.5-84.5° C.
From 4-chlorobenzonitrile (27.5 mg, 0.2 mmol, 1.0 equiv) the title compound was prepared following GP-B and the isolated yield was 12 mg (30%). ¹H NMR (500 MHz, CDCl₃) δ 7.61 (d, J=8.6 Hz, 2H), 7.24 (d, J=8.1 Hz, 2H), 3.67 (dd, J=12.6, 5.4 Hz, 1H), 2.54 (m, 1H), 2.48 (m, 1H), 2.28 (m, 1H), 2.20 (m, 1H), 2.00 (m, 2H), 1.84 (m, 2H). ¹³C NMR (126 MHz, CDCl₃) δ 208.89, 144.24, 132.09, 129.55, 118.93, 110.81, 57.46, 42.23, 35.12, 27.71, 25.33. HRMS exact mass calculated for (C₁₃H₁₄NO⁺) requires m/z 200.1070, found m/z 200.1069

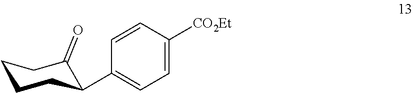

Ethyl-4-(2-oxocyclohexyl)benzoate (13): Prepared according to GP-A using Acr 7 (0.28 mg, 0.0004 mmol, 0.002 equiv, 0.2 mol %), ethyl-4-iodobenzoate (55.2 mg, 0.2 mmol, 1.0 equiv), cyclohexanone (104 μL, 1 mmol, 5.0 equiv), pyrrolidine (84 μL, 1 mmol, 5.0 equiv) and dry MeCN (2 ml, 0.1 M). Workup was performed after 24 h and purification by flash column chromatography (20-25% Et₂O/Hexanes) afforded the title compound as a white solid (35 mg, 70%); mp 85.0-87.0° C.
From ethyl-4-bromobenzoate (45.8 mg, 0.2 mmol, 1.0 equiv) the title compound was prepared following GP-B and the isolated yield was 25 mg (51%).
¹H NMR (500 MHz, CDCl₃) δ 8.01 (d, J=8.5 Hz, 2H), 7.21 (d, J=8.1 Hz, 2H), 4.37 (q, J=7.1 Hz, 2H), 3.67 (dd, J=12.4, 5.2 Hz, 1H), 2.54 (m, 1H), 2.47 (m, 1H), 2.28 (m, 1H), 2.18 (m, 1H), 2.03 (m, 2H), 1.84 (m, 2H), 1.38 (t, J=7.1 Hz, 3H). ¹³C{¹H } NMR (126 MHz, CDCl₃) δ 209.48, 166.51, 143.92, 129.61, 129.16, 128.63, 60.83, 57.43, 42.23, 35.05, 27.75, 25.32, 14.36. HRMS (ESI) exact mass calculated for (C₁₅H₁₉O₃⁺) requires m/z 247.1328, found m/z 247.1327.

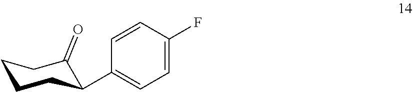

2-(4-fluorophenyl)cyclohexan-1-one (14): Prepared according to GP-A using Acr 7 (0.28 mg, 0.0004 mmol, 0.002 equiv, 0.2 mol %), 4-fluoroiodobenzene (44.4 mg, 0.2 mmol, 1.0 equiv), cyclohexanone (104 μL, 1 mmol, 5.0 equiv), pyrrolidine (84 μL, 1 mmol, 5.0 equiv) and dry MeCN (2 ml, 0.1 M). Workup was performed after 24 h and purification by flash column chromatography (10-15% Et₂O/Hexanes) afforded the title compound as a white solid (24 mg, 63%); mp 56.0-58.0° C. ¹H NMR (500 MHz, CDCl₃) δ 7.10 (m, 2H), 7.02 (m, 2H), 3.60 (dd, J=12.3, 5.4 Hz, 1H), 2.53 (m, 1H), 2.46 (m, 1H), 2.26 (m, 1H), 2.17 (m, 1H), 1.99 (m, 2H), 1.82 (m, 2H). ¹³C{¹H} NMR (126 MHz, CDCl₃) 6 210.08, 162.77, 160.83, 134.47, 134.44, 130.05, 129.99, 115.27, 115.10, 56.68, 42.22, 35.46, 27.83, 25.43. ¹⁹F{¹H} NMR (470 MHz, CDCl₃) δ-116.20, -116.20, -116.21, -116.21, -116.22, -116.23, -116.24. HRMS (ESI) exact mass calculated for (C₁₂K₄FO⁺¹) requires m/z 193.1023, found m/z 193.1024.

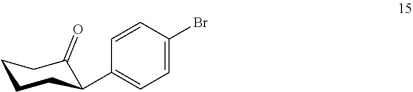

2-(4-bromophenyl)cyclohexan-1-one (15): Prepared according to GP-A using Acr 7 (0.28 mg, 0.0004 mmol, 0.002 equiv, 0.2 mol %), 4-iodobromobenzene (56.6 mg, 0.2 mmol, 1.0 equiv), cyclohexanone (104 μL, 1 mmol, 5.0 equiv), pyrrolidine (84 μL, 1 mmol, 5.0 equiv) and dry MeCN (2 ml, 0.1 M). Workup was performed after 24 h and purification by flash column chromatography (10-15% Et₂O/Hexanes) afforded the title compound as a white solid (34 mg, 67%).
¹H NMR (500 MHz, CDCl₃) δ 7.45 (d, J=8.5 Hz, 2H), 7.01 (d, J=8.4 Hz, 2H), 3.57 (dd, J=12.5, 5.5 Hz, 1H), 2.53 (dddd, J=13.8, 4.6, 3.4, 1.5 Hz, 1H), 2.45 (dddd, J=15.0, 12.7, 5.9, 1.2 Hz, 1H), 2.26 (m, 1H), 2.17 (m, 1H), 1.98 (m, 2H), 1.82 (m, 2H). ¹³C{¹H} NMR (126 MHz, CDCl₃) δ 209.68, 137.74, 131.45, 130.32, 120.86, 56.89, 42.20, 35.22, 27.79, 25.37.

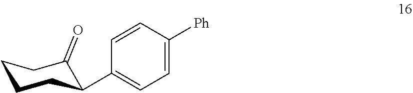

2-([1,1′-biphenyl]-4-yl)cyclohexan-1-one (16): Prepared according to GP-A using Acr 7 (0.28 mg, 0.0004 mmol, 0.002 equiv, 0.2 mol %), 4-iodobiphenyl (56 mg, 0.2 mmol, 1.0 equiv), cyclohexanone (104 μL, 1 mmol, 5.0 equiv), pyrrolidine (84 μL, 1 mmol, 5.0 equiv) and dry MeCN (2 ml, 0.1 M). Workup was performed after 24 h and purification by flash column chromatography (10-15% Et₂O/Hexanes) afforded the title compound as a white solid (33 mg, 66%); mp 105.0-107.0° C.
¹H NMR (500 MHz, CDCl₃) δ 7.59 (m, 4H), 7.43 (t, J=7.6 Hz, 2H), 7.33 (t, J=7.4 Hz, 1H), 7.22 (d, J=8.2 Hz, 2H), 3.67 (dd, J=12.3, 5.4 Hz, 1H), 2.57 (m, 1H), 2.49 (m, 1H), 2.32 (m, 1H), 2.18 (m, 1H), 2.06 (m, 2H), 1.86 (m, 2H). ¹³C{¹H} NMR (126 MHz, CDCl₃) δ 210.33, 141.03, 139.85, 137.85, 128.96, 128.71, 127.17, 127.14, 57.13, 42.26, 35.20, 27.86, 25.40.

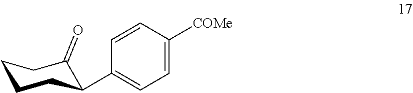

2-(4-acetylphenyl)cyclohexan-1-one (17): Prepared according to GP-A using Acr 7 (0.28 mg, 0.0004 mmol, 0.002 equiv, 0.2 mol %), 4-iodoacetophenone (49.2 mg, 0.2 mmol, 1.0 equiv), cyclohexanone (104 μL, 1 mmol, 5.0 equiv), pyrrolidine (84 μL, 1 mmol, 5.0 equiv) and dry MeCN (2 ml, 0.1 M). Workup was performed after 24 h and purification by flash column chromatography (30% Et₂O/Hexanes) afforded the title compound as a white solid (27 mg, 63%); mp 77.5-79.5° C.
From 4-bromoacetophenone (39.8 mg, 0.2 mmol, 1.0 equiv) the title compound was prepared following GP-B and the isolated yield was 17 mg (40%). ¹H NMR (500 MHz, CDCl₃) δ 7.93 (d, J=8.6 Hz, 2H), 7.24 (d, J=8.1 Hz, 2H), 3.68 (dd, J=12.4, 5.5 Hz, 1H), 2.59 (s, 3H), 2.54 (m, 1H), 2.48 (m, 1H), 2.28 (m, 1H), 2.19 (m, 1H), 2.03 (m, 2H), 1.84 (m, 2H).
¹³C{¹H} NMR (126 MHz, CDCl₃) δ 209.44, 197.82, 144.30, 135.88, 128.90, 128.44, 57.45, 42.35, 42.25, 35.05, 27.76, 26.62, 25.34. HRMS (ESI) exact mass calculated for (C₁₄H₁₆O₂⁺) requires m/z 217.1223, found m/z 217.1224.

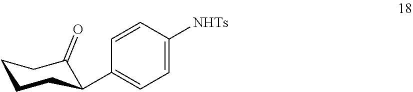

4-methyl-N-(4-(2-oxocyclohexyl)phenyl)benzenesulfonamide (18): Prepared according to GP-B using Acr 7 (2.8 mg, 0.004 mmol, 0.02 equiv, 2.0 mol %), N-(4-iodophenyl)-4-methylbenzenesulfonamide (74.6 mg, 0.2 mmol, 1.0 equiv), cyclohexanone (104 μL, 1 mmol, 5.0 equiv), pyrrolidine (84 μL, 1 mmol, 5.0 equiv) and dry MeCN (2 ml, 0.1 M). Workup was performed after 24 h and purification by flash column chromatography (60% Et₂O/Hexanes) afforded the title compound as a white solid (39 mg, 57%); mp 128.0-130.0° C.
¹H NMR (500 MHz, CDCl₃) δ 7.66 (d, J=8.3 Hz, 2H), 7.22 (m, 2H), 7.00 (m, 4H), 6.80 (s, 1H), 3.54 (ddd, J=12.3, 5.4, 1.1 Hz, 1H), 2.50 (dddd, J=13.8, 4.9, 3.6, 1.4 Hz, 1H), 2.43 (m, 1H), 2.37 (s, 3H), 2.17 (m, 2H), 1.95 (m, 2H), 1.79 (m, 2H). ¹³C{¹H}NMR (126 MHz, CDCl₃) δ 210.35, 143.80, 136.34, 135.79, 135.19, 129.65, 129.43, 127.26, 121.46, 56.74, 42.21, 35.16, 27.79, 25.33, 21.54. HRMS (ESI) exact mass calculated for (C₁₉H₂₁NO₃S⁺) requires m/z 366.1134, found m/z 366.1134.

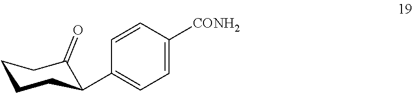

4-(2-oxocyclohexyl)benzamide (19): Prepared according to GP-B using Acr 7 (2.8 mg, 0.004 mmol, 0.02 equiv, 2.0 mol %), 4-iodobenzamide (49.4 mg, 0.2 mmol, 1.0 equiv), cyclohexanone (104 μL, 1 mmol, 5.0 equiv), pyrrolidine (84 μL, 1 mmol, 5.0 equiv) and dry MeCN (2 ml, 0.1 M). Workup was performed after 24 h and purification by flash column chromatography (60% EtOAc/Hexanes) afforded the title compound as a white solid (29 mg, 67%).
¹H NMR (500 MHz, CD₂Cl₂/DMSO-d₆) δ 7.76 (d, J=8.3 Hz, 2H), 7.27 (s, 1H), 7.12 (d, J=8.1 Hz, 2H), 6.31 (s, 1H), 3.62 (dd, J=12.5, 5.3 Hz, 1H), 2.41 (m, 2H), 2.19 (m, 1H), 2.10 (m, 1H), 1.93 (m, 2H), 1.76 (m, 2H). ¹³C{¹H} NMR (126 MHz, CD₂Cl₂/DMSO-d₆) δ 209.53, 168.91, 143.07, 132.85, 128.80, 127.65, 57.31, 42.37,35.21, 27.90, 25.50. HRMS (ESI) exact mass calculated for (C₁₃H₁₆NO₂⁺) requires m/z 218.1176, found m/z 218.1174.

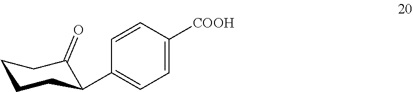

4-(2-oxocyclohexyl)benzoic acid (20): Prepared according to GP-B using Acr 7 (2.8 mg, 0.004 mmol, 0.02 equiv, 2.0 mol %), 4-iodobenzoic acid (49.6 mg, 0.2 mmol, 1.0 equiv), cyclohexanone (104 μL, 1 mmol, 5.0 equiv), pyrrolidine (84 μL, 1 mmol, 5.0 equiv) and dry MeCN (2 ml, 0.1 M). Workup was performed after 24 h and purification by flash column chromatography (10:2:0.2 Hexanes:EtOAc:AcOH) afforded the title compound as a white solid (25 mg, 57%).
¹H NMR (500 MHz, CDCl₃) δ 12.18 (brs, 1H), 8.08 (d, J=8.3 Hz, 2H), 7.25 (d, J=8.6 Hz, 2H), 3.69 (dd, J=12.3, 5.4 Hz, 1H), 2.52 (m, 2H), 2.29 (m, 2H), 2.19 (m, 1H), 2.04 (m, 2H), 1.85 (m, 1H). ¹³C{¹H} NMR (126 MHz, CDCl₃) δ 209.50, 171.92, 145.03, 130.29, 128.84, 127.97, 57.51, 42.23, 35.07, 27.75, 25.31. HRMS (ESI) exact mass calculated for (C₁₃H₁₅O₃⁺) requires m/z 219.1016, found m/z 219.1014.

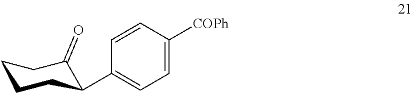

2-(4-benzoylphenyl)cyclohexan-1-one (21): Prepared according to GP-B using Acr 7 (2.8 mg, 0.004 mmol, 0.02 equiv, 2.0 mol %), 4-bromobenzophenone (52.2 mg, 0.2 mmol, 1.0 equiv), cyclohexanone (104 μL, 1 mmol, 5.0 equiv), pyrrolidine (84 μL, 1 mmol, 5.0 equiv) and dry MeCN (2 ml, 0.1 M). Workup was performed after 24 h and purification by flash column chromatography (30-35% Et₂O/Hexanes) afforded the title compound as a white solid (28 mg, 50%).
From 4-chlorobenzophenone (43.3 mg, 0.2 mmol, 1.0 equiv) the title compound was prepared following GP-B and the isolated yield was 15 mg (27%). 1H NMR (500 MHz, CDCl₃) δ 7.80 (m, 4H), 7.58 (m, 1H), 7.48 (m, 2H), 7.26 (d, J=8.0 Hz, 2H), 3.70 (dd, J=12.3, 5.0 Hz, 1H), 2.56 (m, 1H), 2.49 (m, 1H), 2.31 (m, 1H), 2.19 (m, 1H), 2.04 (m, 2H), 1.85 (m, 2H).
¹³C{¹H} NMR (126 MHz, CDCl₃) δ 209.53, 196.39, 143.70, 137.77, 136.15, 132.27, 130.24, 130.01, 128.63, 128.25, 57.47, 42.27, 35.21, 27.77, 25.35.

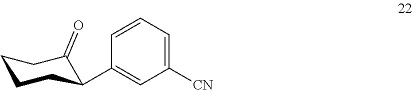

3-(2-oxocyclohexyl)benzonitrile (22): Prepared according to GP-A using Acr 7 (0.7 mg, 0.001 mmol, 0.002 equiv, 0.2 mol %), 3-iodobenzonitrile (114.5 mg, 0.5 mmol, 1.0 equiv), cyclohexanone (260 μL, 2.5 mmol, 5.0 equiv), pyrrolidine (210 μL, 2.5 mmol, 5.0 equiv) and dry MeCN (5 ml, 0.1 M). Workup was performed after 24 h and purification by flash column chromatography (10-20% Et₂O/Hexanes) afforded the title compound as a white solid (72 mg, 72%); mp 93.0-95.0° C.
From 3-iodobenzonitrile (36.4 mg, 0.2 mmol, 1.0 equiv) the title compound was prepared following GP-B and the isolated yield was 19 mg (48%).
¹H NMR (500 MHz, CDCl₃) δ 7.54 (m, 1H), 7.42 (m, 2H), 7.37 (ddd, J=7.9, 2.1, 1.3 Hz, 1H), 3.64 (dd, J=12.6, 5.4 Hz, 1H), 2.51 (m, 2H), 2.28 (m, 1H), 2.20 (m, 1H), 2.00 (m, 2H), 1.83 (m, 2H). ¹³C{¹H} NMR (126 MHz, CDCl₃) δ 208.93, 140.17, 133.35, 132.26, 130.59, 129.01, 118.88, 112.36, 56.89, 42.16, 35.18, 27.68, 25.32. HRMS exact mass calculated for (C₁₃H₁₄NO⁺) requires m/z 200.1070, found m/z 200.1070.

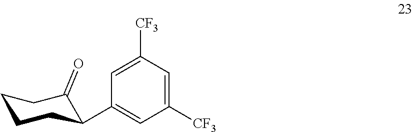

2-(3,5-bis(trifluoromethyl)phenyl)cyclohexan-1-one (23): Prepared according to GP-A using Acr 7 (0.7 mg, 0.001 mmol, 0.002 equiv, 0.2 mol %), 3,5-bistrifluoromethyl iodobenzene (170 mg, 0.5 mmol, 1.0 equiv), cyclohexanone (260 μL, 2.5 mmol, 5.0 equiv), pyrrolidine (210 μL, 2.5 mmol, 5.0 equiv) and dry MeCN (5 ml, 0.1 M). Workup was performed after 12 h and purification by flash column chromatography (10-20% Et₂O/Hexanes) afforded the title compound as a white solid (113 mg, 73%).
¹H NMR (500 MHz, CDCl₃) δ 7.78 (s, 1H), 7.59 (s, 2H), 3.76 (dd, J=12.8, 5.4 Hz, 1H), 2.57 (m, 1H), 2.50 (m, 1H), 2.33 (ddt, J=14.2, 5.9, 3.1 Hz, 1H), 2.22 (dddd, J=11.9, 9.0, 5.1, 2.3 Hz, 1H), 2.03 (m, 2H), 1.85 (m, 2H). ¹³C{¹H} NMR (126 MHz, CDCl₃) δ 208.38, 141.19, 131.41 (q, J=33.14 Hz), 129.06 (q, J=2.51 Hz), 123.41 (q, J=272.66 Hz), 120.98 (q, J=3.85 Hz), 57.04, 42.18, 35.35, 27.67, 25.36. ¹⁹F{¹H} NMR (470 MHz, CDCl₃) δ-62.83.

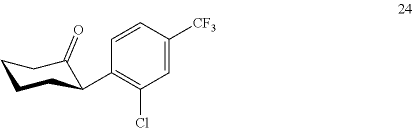

2-(2-chloro-4-(trifluoromethyl)phenyl)cyclohexan-1-one (24): Prepared according to GP-B using Acr 7 (7.0 mg, 0.01 mmol, 0.02 equiv, 2.0 mol %), 2-chloro-1-iodo-4-(trifluoromethyl)benzene (153 mg, 0.5 mmol, 1.0 equiv), cyclohexanone (260 μL, 2.5 mmol, 5.0 equiv), pyrrolidine (210 μL, 2.5 mmol, 5.0 equiv) and dry MeCN (5 ml, 0.1 M). Workup was performed after 48 h and purification by flash column chromatography (10-20% Et₂O/Hexanes) afforded the title compound as a white solid (86 mg, 62%); mp 78.0-80.0° C.
¹H NMR (500 MHz, CDCl₃) δ 7.65 (m, 1H), 7.52 (m, 1H), 7.35 (d, J=8.2 Hz, 1H), 4.15 (dd,J=12.8, 5.2 Hz, 1H), 2.57 (m, 2H), 2.26 (m, 2H), 2.07 (m, 1H), 2.00 (td, J=12.6, 3.2 Hz, 1H), 1.86 (m, 2H). ¹³C{¹H} NMR (126 MHz, CDCl₃) δ 207.95, 140.80, 134.73, 130.46 (q, J=33.16 Hz), 129.97, 126.39 (q, J=3.90 Hz), 123.59 (q, J=3.70 Hz), 123.34 (q, J=272.30 Hz), 53.95, 42.30, 33.87, 27.60, 25.53. ¹⁹F{¹H} NMR (470 MHz, CDCl₃) δ-62.77. HRMS exact mass calculated for (C₁₃H₁₃ClF₃O⁺) requires m/z 277.0602, found m/z 277.0602.

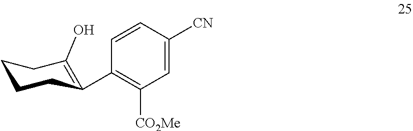

Methyl 4-cyano-6′-hydroxy-2′,3′,4′,5′-tetrahydro-[1,1′-biphenyl]-2-carboxylate (25): Prepared according to GP-B using Acr 7 (7.0 mg, 0.01 mmol, 0.02 equiv, 2.0 mol %), methyl 2-bromo-5-cyanobenzoate (120 mg, 0.5 mmol, 1.0 equiv), cyclohexanone (260 μL, 2.5 mmol, 5.0 equiv), pyrrolidine (210 μL, 2.5 mmol, 5.0 equiv) and dry MeCN (5 ml, 0.1 M). Workup was performed after 6 h and purification by flash column chromatography (10-20% Et₂O/Hexanes) afforded the title compound as a white solid (99 mg, 77%); mp 136.0-138.0° C. Only enol from of this compound was observed and isolated. No ketone form was detected in crude reaction mixture also (by ¹H NMR spectroscopy).
¹H NMR (500 MHz, CDCl₃) δ 7.84 (d, J=2.1 Hz, 1H), 7.47 (dd, J=8.9, 2.1 Hz, 1H), 6.73 (d, J=8.9 Hz, 1H), 3.89 (s, 3H), 3.27 (m, 4H), 1.98 (m, 4H). ¹³C{¹H} NMR (126 MHz, CDCl₃) δ 167.58, 149.51, 135.97, 134.49, 119.69, 116.52, 114.24, 97.08, 52.43, 50.92, 25.76.

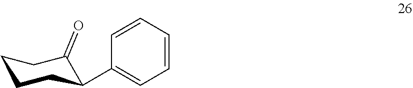

2-phenylcyclohexan-1-one (26): Prepared according to GP-A using Acr 7 (0.28 mg, 0.0004 mmol, 0.002 equiv, 0.2 mol %), 4-iodobenzene (40.8 mg, 0.2 mmol, 1.0 equiv), cyclohexanone (104 μL, 1 mmol, 5.0 equiv), pyrrolidine (84 μL, 1 mmol, 5.0 equiv) and dry MeCN (2 ml, 0.1 M). Workup was performed after 24 h and purification by flash column chromatography (10-15% Et₂O/Hexanes) afforded the title compound as a white solid (21 mg, 61%).
¹H NMR (500 MHz, CDCl₃) δ 7.34 (t, J=7.4 Hz, 2H), 7.26 (t, J=7.4 Hz, 1H), 7.15 (d, J=7.3 Hz, 2H), 3.62 (dd, J=12.4, 5.5 Hz, 1H), 2.54 (dtd, J=13.7, 4.1, 1.4 Hz, 1H), 2.46 (m, 1H), 2.28 (m, 1H), 2.16 (m, 1H), 2.03 (m, 1H), 1.84 (m, 1H). ¹³C{¹H} NMR (126 MHz, CDCl₃) δ 210.28, 138.77, 128.53, 128.36, 126.90, 57.40, 42.20, 35.10, 27.83, 25.34.

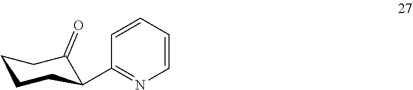

2-(pyridin-2-yl)cyclohexan-1-one (27): Prepared according to GP-A using Acr 7 (0.28 mg, 0.0004 mmol, 0.002 equiv, 0.2 mol %), 2-iodopyridine (41 mg, 0.2 mmol, 1.0 equiv), cyclohexanone (104 μL, 1 mmol, 5.0 equiv), pyrrolidine (84 μL, 1 mmol, 5.0 equiv) and dry MeCN (2 ml, 0.1 M). Workup was performed after 24 h and purification by flash column chromatography (15-20% Et₂O/Hexanes) afforded the title compound as a keto-enol mixture (light yellow solid; 20 mg, 57%).
Enol characterization: ¹H NMR (500 MHz, CDCl₃) δ 15.53 (s, 1H), 8.32 (ddd, J=5.2, 1.9, 1.0 Hz, 1H), 7.67 (ddd, J=8.3, 7.4, 1.9 Hz, 1H), 7.11 (d, J=8.4 Hz, 1H), 7.00 (ddd, J=7.4, 5.1, 1.1 Hz, 1H), 2.36 (dddd, J=12.9, 6.6, 5.0, 1.8 Hz, 4H), 1.76 (m, 4H). ¹³C NMR (126 MHz, CDCl₃) 8163.18, 160.06, 144.57, 137.17, 118.43, 116.86, 100.53, 30.18, 24.58, 23.13, 22.47.
Ketone characterization: ¹H NMR (500 MHz, CDCl₃) δ 8.56 (dd, J=5.6, 1.9 Hz, 1H), 7.66 (ddd, J=8.4, 7.6, 1.9 Hz, 1H), 7.17 (ddd, J=7.7, 3.6, 1.5 Hz, 2H), 3.84 (ddd, J=11.7, 5.6, 1.1 Hz, 1H), 2.56 (dtd, J=14.0, 4.4, 1.3 Hz, 1H), 2.48 (dddd, J=13.9, 11.9, 5.9, 1.2 Hz, 1H), 2.31 (m, 1H), 2.16 (m, 2H), 2.01 (m, 1H), 1.84 (m, 2H). ¹³C{¹H} NMR (126 MHz, CDCl₃) δ 210.02, 158.69, 149.23, 136.24, 123.38, 121.92, 59.37, 42.16, 33.55, 27.58, 24.76.

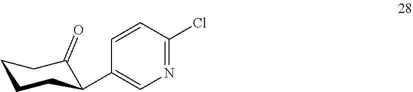

2-(6-chloropyridin-3-yl)cyclohexan-1-one (28): Prepared according to GP-A using Acr 7 (0.28 mg, 0.0004 mmol, 0.002 equiv, 0.2 mol %), 2-chloro-5-iodopyridine (47.9 mg, 0.2 mmol, 1.0 equiv), cyclohexanone (104 μL, 1 mmol, 5.0 equiv), pyrrolidine (84 μL, 1 mmol, 5.0 equiv) and dry MeCN (2 ml, 0.1 M). Workup was performed after 24 h and purification by flash column chromatography (30% Et₂O/Hexanes) afforded the title compound as a waxy white solid (31 mg, 74%). ¹H NMR (500 MHz, CDCl₃) δ 8.14 (d, J=2.5 Hz, 1H), 7.46 (dd, J=8.2, 2.5 Hz, 1H), 7.30 (d, J=8.2 Hz, 1H), 3.63 (dd, J=12.5, 5.4 Hz, 1H), 2.56 (m, 1H), 2.49 (m, 1H), 2.28 (ddt,J=13.8, 5.4, 2.9 Hz, 1H), 2.22 (dtd, J=11.5, 5.9, 3.1 Hz, 1H), 2.03 (m, 1H), 1.87 (m, 3H). ¹³C{¹H} NMR (126 MHz, CDCl₃) δ 208.68, 150.06, 149.64, 139.17, 133.20, 123.90, 54.22, 42.19, 35.53, 27.77, 25.44.

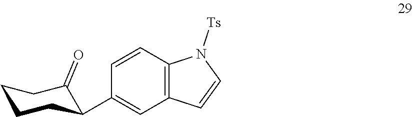

2-(1-tosyl-1H-indol-5-yl)cyclohexan-1-one (29): Prepared according to GP-A using Acr 7 (0.28 mg, 0.0004 mmol, 0.002 equiv, 0.2 mol %), 5-iodo-1-tosyl-1H-indole (79.4 mg, 0.2 mmol, 1.0 equiv), cyclohexanone (104 μL, 1 mmol, 5.0 equiv), pyrrolidine (84 μL, 1 mmol, 5.0 equiv) and dry MeCN (2 ml, 0.1 M). Workup was performed after 24 h and purification by flash column chromatography (40% Et₂O/Hexanes) afforded the title compound as a white solid (46 mg, 62%); mp 143.0-145.0° C.
¹H NMR (500 MHz, CDCl₃) δ 7.92 (d, J=8.5 Hz, 1H), 7.77 (d, J=8.5 Hz, 2H), 7.53 (d, J=3.7 Hz, 1H), 7.30 (d, J=1.7 Hz, 1H), 7.22 (d, J=8.1 Hz, 2H), 7.07 (dd, J=8.5, 1.7 Hz, 1H), 6.60 (dd, J=3.7, 0.8 Hz, 1H), 3.67 (dd, J=12.3, 5.4 Hz, 1H), 2.53 (m, 1H), 2.46 (m, 1H), 2.34 (s, 3H), 2.27 (m, 1H), 2.16 (m, 1H), 2.02 (m, 2H), 1.82 (m, 2H). ¹³C{¹H} NMR (126 MHz, CDCl₃) δ 210.62, 144.93, 135.40, 133.87, 133.84, 130.90, 129.92, 126.90, 126.40, 125.40, 121.07, 113.31, 108.96, 57.31, 42.25, 35.67, 27.84, 25.47, 21.56. HRMS (ESI) exact mass calculated for (C₂₁H₂₁NO₃S⁺) requires m/z 406.0874, found m/z 406.0874.

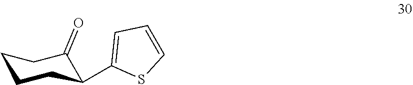

2-(thiophen-2-yl)cyclohexan-1-one (30): Prepared according to GP-A using Acr 7 (7.0 mg, 0.01 mmol, 0.002 equiv, 0.2 mol %), 2-iodothiophene (1.05 g, 5.0 mmol, 1.0 equiv), cyclohexanone (0.52 mL, 25.0 mmol, 5.0 equiv), pyrrolidine (0.42 mL, 25.0 mmol, 5.0 equiv) and dry MeCN (50 ml, 0.1 M). Workup was performed after 48 h and purification by flash column chromatography (5% Et₂O/Hexanes) afforded the title compound as a colorless oil (405 mg, 45%).
¹H NMR (500 MHz, CDCl₃) δ 7.24 (dd, J=5.1, 1.2 Hz, 1H), 6.99 (dd, J=5.1, 3.5 Hz, 1H), 6.86 (d, J=3.3 Hz, 1H), 3.92 (dd, J=11.2, 5.5 Hz, 1H), 2.56 (dtd, J=13.7, 4.4, 1.3 Hz, 1H), 2.43 (m, 2H), 2.05 (m, 3H), 1.83 (m, 2H). ¹³C{¹H} NMR (126 MHz, CDCl₃) δ 208.91, 141.11, 126.53, 125.12, 124.39, 52.05, 41.66, 36.16, 27.68, 24.95.

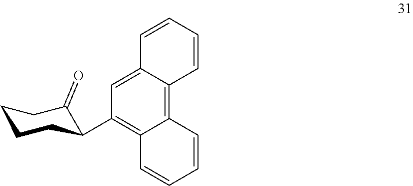

2-(phenanthren-9-yl)cyclohexan-1-one (31): Prepared according to GP-B using Acr 7 (2.8 mg, 0.004 mmol, 0.02 equiv, 2.0 mol %), 9-bromophenanthrene(51.5 mg, 0.2 mmol, 1.0 equiv), cyclohexanone (104 μL, 1 mmol, 5.0 equiv), pyrrolidine (84 μL, 1 mmol, 5.0 equiv) and dry MeCN (2 ml, 0.1 M). Workup was performed after 24 h and purification by flash column chromatography (40% Et₂O/Hexanes) afforded the title compound as a light yellow solid (18 mg, 33%). NMR (500 MHz, CDCl₃) δ 8.75 (d, J=8.1 Hz, 1H), 8.67 (d, J=8.2 Hz, 1H), 7.84 (d, J=8.8 Hz, 1H), 7.73 (d, J=8.2 Hz, 1H), 7.60 (m, 5H), 4.39 (dd, J=12.5, 5.1 Hz, 1H), 2.67 (m, 2H), 2.52 (m, 1H), 2.40 (qd, J=12.6, 3.5 Hz, 1H), 2.30 (ddd, J=13.1, 6.1, 3.0 Hz, 1H), 2.19 (m, 1H), 1.97 (m, 2H). ¹³C{¹H} NMR (126 MHz, CDCl₃) δ 210.22, 133.62, 131.56, 130.97, 130.69, 130.01, 128.56, 126.59, 126.49, 126.48, 126.12, 124.04, 123.40, 122.45, 42.80, 33.96, 28.12, 26.02.

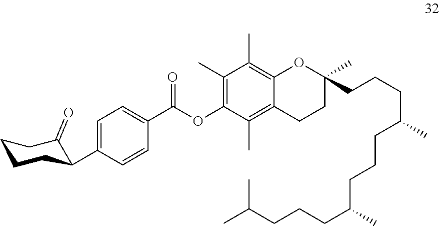

(S)-2,5,7,8-tetramethyl-24(4R,8R)-4,8,12-trimethyltridecyl)chroman-6-yl-4-(-2-oxocyclohexyl) benzoate (32): Prepared according to GP-A using Acr 7 (0.28 mg, 0.0004 mmol, 0.002 equiv, 0.2 mol %), (S)-2,5,7,8-tetramethyl-2-((4R,8R)-4,8,12-trimethyltridecyl)chroman-6-yl 4-iodobenzoate (132 mg, 0.2 mmol, 1.0 equiv), cyclohexanone (104 μL, 1 mmol, 5.0 equiv), pyrrolidine (84 μL, 1 mmol, 5.0 equiv) and dry MeCN (2 ml, 0.1 M). Workup was performed after 24 h and purification by flash column chromatography (10-20% Et₂O/Hexanes) afforded the title compound as a mixture of diastereomers (d.r. not determined). White solid (83 mg, 66% yield).
¹H NMR (500 MHz, CDCl₃) δ 8.22 (d, J=8.3 Hz, 2H), 7.29 (d, J=8.3 Hz, 2H), 3.72 (dd, J=12.4, 5.4 Hz, 1H), 2.59 (m, 3H), 2.50 (m, 1H), 2.33 (m, 1H), 2.21 (m, 1H), 2.12 (s, 3H), 2.05 (m, 4H), 2.01 (s, 3H), 1.85 (m, 4H), 1.58-1.04 (m, 25H), 0.86 (m, 12H). ¹³C{¹H} NMR (126 MHz, CDCl₃) δ 209.47, 165.01, 149.44, 144.59, 140.65, 130.28, 128.89, 128.33, 126.97, 125.19, 123.10, 117.46, 75.07, 57.56, 42.27, 39.38, 37.47, 37.30, 35.15, 32.82, 27.99, 27.77, 25.38, 24.82, 24.47, 24.21, 23.72, 22.73, 22.64, 21.05, 20.64, 19.76, 19.67, 13.07, 12.22, 11.85.

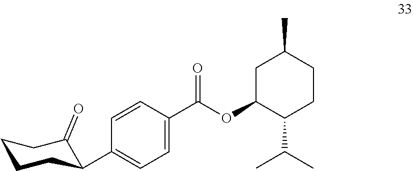

(1S,2R,5S)-2-isopropyl-5-methylcyclohexyl-4-(2-oxocyclohexyl)benzoate (33): Prepared according to GP-A using Acr 7 (0.28 mg, 0.0004 mmol, 0.002 equiv, 0.2 mol %), (1S,2R,5S)-2-isopropyl-5-methylcyclohexyl 4-iodobenzoate (77.3 mg, 0.2 mmol, 1.0 equiv), cyclohexanone (104 μL, 1 mmol, 5.0 equiv), pyrrolidine (84 μL, 1 mmol, 5.0 equiv) and dry MeCN (2 ml, 0.1 M). Workup was performed after 24 h and purification by flash column chromatography (15% Et₂O/Hexanes) afforded the title compound as a mixture of diastereomers (d.r. not determined). White solid (48 mg, 67% yield); mp 105.0-107.0° C. 1H NMR (500 MHz, CDCl₃) δ 8.01 (d, J=8.4 Hz, 2H), 7.21 (d, J=8.3 Hz, 2H), 4.92 (td, J=10.8, 4.4 Hz, 1H), 3.67 (dd, J=12.1, 5.2 Hz, 1H), 2.54 (dddd, J=14.0, 4.7, 3.5, 1.4 Hz, 1H), 2.47 (m, 1H), 2.28 (m, 1H), 2.17 (m, 1H), 2.13-1.92 (m, 4H), 1.83 (m, 2H), 1.72 (m, 2H), 1.54 (m, 2H), 1.10 (m, 2H), 0.91 (m, 6H), 0.78 (d, J=6.9 Hz, 3H). ¹³C{¹H} NMR (126 MHz, CDCl₃) δ 209.50, 165.94, 143.83, 143.81, 129.65, 129.53, 128.61, 74.67, 57.46, 47.30, 42.23, 40.98, 35.12, 35.00, 34.35, 31.46, 27.74, 26.47, 25.34, 25.33, 23.63, 22.06, 20.79, 16.50. HRMS (ESI) exact mass calculated for (C₂₃H₃₃O₃⁺) requires m/z 357.2424, found m/z 357.2414.

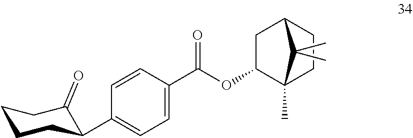

(1S,2R,4S)-1,7,7-trimethylbicyclo[2.2.1]heptan-2-yl 4-(2-oxocyclohexyl)benzoate (34): Prepared according to GP-A using Acr 7 (0.28 mg, 0.0004 mmol, 0.002 equiv, 0.2 mol %), (1S,2R,4S)-1,7,7-trimethylbicyclo[2.2.1]heptan-2-yl 4-ethylbenzoate (76.9 mg, 0.2 mmol, 1.0 equiv), cyclohexanone (104 μL, 1 mmol, 5.0 equiv), pyrrolidine (84 μL, 1 mmol, 5.0 equiv) and dry MeCN (2 ml, 0.1 M). Workup was performed after 24 h and purification by column chromatography (15% Et₂O/Hexanes) afforded the title compound as a mixture of diastereomers (d.r. not determined). White solid (50 mg, 70% yield). ¹H NMR (500 MHz, CDCl₃) δ 8.03 (d, J=8.3 Hz, 2H), 7.22 (d, J=8.2 Hz, 2H), 5.11 (dddd, J=9.9, 3.5, 2.2, 1.3 Hz, 1H), 3.68 (dd, J=11.9, 5.4 Hz, 1H), 2.54-2.44 (m, 3H), 2.29 (m, 1H), 2.16 (m, 2H), 2.03 (m, 2H), 1.82 (m, 3H), 1.73 (t, J=4.5 Hz, 1H), 1.40 (m, 1H), 1.30 (m, 1H), 1.10 (dd, J=13.8, 3.5 Hz, 1H), 0.96 (s, 3H), 0.91 (s, 3H), 0.90 (s, 3H). ¹³C{¹H} NMR (126 MHz, CDCl₃) δ 209.48, 166.68, 143.88, 129.61, 129.58, 128.64, 80.38, 57.46, 49.10, 47.88, 45.01, 42.24, 36.94, 35.04, 28.10, 27.76, 27.40, 25.34, 19.74, 18.93, 13.60. HRMS (ESI) exact mass calculated for (C₂₃H₃₀O₃K⁺) requires m/z 393.1827, found m/z 393.1827.

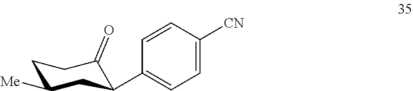

4-(5-methyl-2-oxocyclohexyl)benzonitrile (35): Prepared according to GP-B using Acr 7 (2.8 mg, 0.004 mmol, 0.02 equiv, 2.0 mol %), 4-iodobenzonitrile (45.8 mg, 0.2 mmol, 1.0 equiv), 4-methylcyclohexanone (124 μL, 1 mmol, 5.0 equiv), pyrrolidine (84 μL, 1 mmol, 5.0 equiv) and dry MeCN (2 ml, 0.1 M). Workup was performed after 24 h and purification by flash column chromatography (15-20% Et₂O/Hexanes) afforded the title compound as a white solid (34.6 mg, 81%, 6.5:1 d.r. determined by crude ¹H NMR in CDCl₃); mp 77.0-79.0° C.
¹H NMR (500 MHz, CDCl₃) δ 7.62 (d, J=8.4 Hz, 2H), 7.23 (d, J=8.2 Hz, 2H), 3.72 (dd, J=13.4, 5.2 Hz, 1H), 2.54 (m, 2H), 2.21 (ddt,J=12.8, 5.2, 3.3 Hz, 1H), 2.15 (m, 2H), 1.71 (td, J=13.1, 11.3 Hz, 1H), 1.55 (m, 1H), 1.07 (d, J=6.3 Hz, 3H). ¹³C{¹H} NMR (126 MHz, CDCl₃) δ 208.98, 144.19, 132.08, 129.61, 118.92, 110.82, 56.63, 43.29, 41.51, 35.65, 32.26, 21.19. HRMS (ESI) exact mass calculated for (C₁₄H₁₆NO⁺) requires m/z 214.1226, found m/z 214.1227.

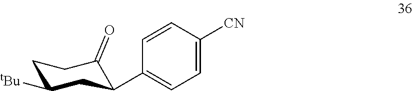

4-(5-(tert-butyl)-methyl-2-oxocyclohexyl)benzonitrile (36): Prepared according to GP-B using Acr 7 (2.8 mg, 0.004 mmol, 0.02 equiv, 2.0 mol %), 4-iodobenzonitrile (45.8 mg, 0.2 mmol, 1.0 equiv), 4-tert-butylcyclohexanone (154.3 mg, 1 mmol, 5.0 equiv), pyrrolidine (84 μL, 1 mmol, 5.0 equiv) and dry MeCN (2 ml, 0.1 M). Workup was performed after 24 h and purification by flash column chromatography (20% Et₂O/Hexanes) afforded the title compound as a white solid (39 mg, 76%, 7:1 d.r. determined by crude NMR in CDCl₃).
¹H NMR (500 MHz, CDCl₃) δ 7.63 (d, J=8.5 Hz, 2H), 7.24 (d, J=8.1 Hz, 2H), 3.67 (dd, J=12.9, 5.0 Hz, 1H), 2.54 (m, 2H), 2.24 (m, 2H), 1.75 (m, 2H), 1.62 (m, 1H), 0.95 (s, 9H). ¹³C{¹H} NMR (126 MHz, CDCl₃) δ 209.17, 144.47, 132.09, 129.67, 118.92, 110.82, 56.89, 47.29, 41.60, 36.60, 32.59, 28.50, 27.62. HRMS (ESI) exact mass calculated for (C₁₇H₂₂NO⁺) requires m/z 256.1696, found m/z 256.1695.

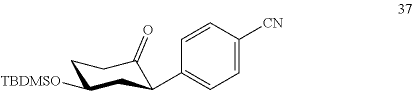

4-(5-((tert-butyldimethylsilyl)oxy)-2-oxocyclohexyl)benzonitrile (37): Prepared according to GP-B using Acr 7 (2.8 mg, 0.004 mmol, 0.02 equiv, 2.0 mol %), 4-iodobenzonitrile (45.8 mg, 0.2 mmol, 1.0 equiv), 4-(tert-butyldimethylsilyloxy)cyclohexanone (228.41, 1 mmol, 5.0 equiv), pyrrolidine (84 μL, 1 mmol, 5.0 equiv) and dry MeCN (2 ml, 0.1 M). Workup was performed after 24 h and purification by flash column chromatography (15% Et₂O/Hexanes) afforded the title compound as a white solid (50 mg, 76%, 9:1 d.r. determined by crude ¹H NMR in CDCl₃); mp 126.0-128.0° C.
¹H NMR (500 MHz, CDCl₃) δ 7.62 (d, J=8.5 Hz, 2H), 7.23 (d, J=8.0 Hz, 2H), 4.31 (tt, J=4.8, 1.5 Hz, 1H), 4.21 (dd, J=13.2, 5.4 Hz, 1H), 2.94 (tdt, J=13.9, 6.2, 0.8 Hz, 1H), 2.37 (dddd,J=13.8, 4.7, 2.4, 0.7 Hz, 1H), 2.23 (ddt, J=13.3, 5.5, 3.4 Hz, 1H), 2.14 (m, 2H), 1.99 (tdd, J=13.8, 4.7, 2.3 Hz, 1H), 0.96 (s, 9H), 0.13 (d, J=7.5 Hz, 6H). ¹³C{¹H} NMR (126 MHz, CDCl₃) δ 209.02, 143.99, 132.14, 129.78, 118.99, 118.94, 110.83, 77.31, 77.05, 76.80, 65.40, 51.34, 42.33, 36.89, 34.78, 25.80, 18.11, -4.89. HRMS (ESI) exact mass calculated for (C₁₉H₂₈NO₂Si⁺) requires m/z 330.1884, found m/z 330.1884.

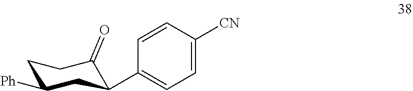

4-(5-phenyl-2-oxocyclohexyl)benzonitrile (38): Prepared according to GP-B using Acr 7 (2.8 mg, 0.004 mmol, 0.02 equiv, 2.0 mol %), 4-iodobenzonitrile (45.8 mg, 0.2 mmol, 1.0 equiv), 4-phenylcyclohexanone (174.3 mg, 1 mmol, 5.0 equiv), pyrrolidine (84 μL, 1 mmol, 5.0 equiv) and dry MeCN (2 ml, 0.1 M). Workup was performed after 24 h and purification by flash column chromatography (15-20% Et₂O/Hexanes) afforded the title compound as a white solid (35 mg, 64%, 7:1 d.r. determined by crude NMR in CDCl₃).
¹H NMR (500 MHz, CDCl₃) δ 7.58 (m, 4H), 7.43 (t, J=7.7 Hz, 2H), 7.34 (t, J=7.4 Hz, 1H), 7.22 (d, J=8.2 Hz, 2H), 3.67 (dd, J=12.3, 5.4 Hz, 1H), 2.57 (m, 1H), 2.49 (m, 1H), 2.32 (m 1H), 2.18 (m, 1H), 2.06 (m, 2H), 1.86 (m, 2H). ¹³C{¹H} NMR (126 MHz, CDCl₃) δ 210.33, 141.03, 139.85, 137.85, 128.96, 128.71, 127.17, 127.14, 57.13, 42.26, 35.20, 27.86, 25.40. HRMS (ESI) exact mass calculated for (C₁₉H₁₇NO⁺) requires m/z 276.1383, found m/z 276.1383.

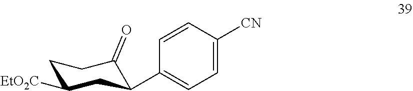

Ethyl 3-(4-cyanophenyl)-4-oxocyclohexane-1-carboxylate (39): Prepared according to GP-B using Acr 7 (2.8 mg, 0.004 mmol, 0.02 equiv, 2.0 mol %), 4-iodobenzonitrile (45.8 mg, 0.2 mmol, 1.0 equiv), ethyl 4-oxocyclohexanecarboxylate (160 μL, 1 mmol, 5.0 equiv), pyrrolidine (84 μL, 1 mmol, 5.0 equiv) and dry MeCN (2 ml, 0.1 M). Workup was performed after 24 h and purification by flash column chromatography (40% Et₂O/Hexanes) and recrystallization from Et₂O afforded the title compound as white needles (39 mg, 75%, 9:1 d.r. determined by crude 1H NMR in CDCl₃); mp 127.0-129.0° C.
¹H NMR (500 MHz, CDCl₃) δ 7.63 (d, J=8.4 Hz, 2H), 7.24 (d, J=8.3 Hz, 2H), 4.16 (q, J=7.1 Hz, 2H), 3.72 (dd, J=13.6, 5.3 Hz, 1H), 2.98 (tt, J=12.3, 3.5 Hz, 1H), 2.63 (ddd, J=14.3, 5.0, 2.8 Hz, 1H), 2.58 (ddd, J=13.6, 5.9, 0.9 Hz, 1H), 2.51 (ddt, J=13.3, 5.3, 3.4 Hz, 1H), 2.45 (ddq, J=13.6, 6.2, 3.1 Hz, 1H), 2.17 (td, J=13.5, 12.3 Hz, 1H), 2.02 (tdd, J=13.5, 12.3, 5.0 Hz, 1H), 1.26 (t, J=7.1 Hz, 3H). ¹³C{¹H} NMR (126 MHz, CDCl₃) δ 206.94, 173.39, 143.16, 132.19, 129.64, 118.78, 111.19, 61.02, 55.74, 42.21, 40.62, 36.74, 29.60, 14.18. HRMS (ESI) exact mass calculated for (C₁₆H₁₈NO₃⁺) requires m/z 272.1281, found m/z 272.1284.

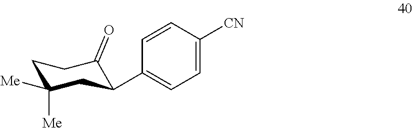

4-(5,5-dimethyl-2-oxocyclohexyl)benzonitrile (40): Prepared according to GP-B using Acr 7 (2.8 mg, 0.004 mmol, 0.02 equiv, 2.0 mol %), 4-iodobenzonitrile (45.8 mg, 0.2 mmol, 1.0 equiv), 4,4-dimethylcyclohexanone (126.2 mg, 1 mmol, 5.0 equiv), pyrrolidine (84 μL, 1 mmol, 5.0 equiv) and dry MeCN (2 ml, 0.1 M). Workup was performed after 24 h and purification by flash column chromatography (25-30% Et₂O/Hexanes) afforded the title compound as a white solid (29 mg, 64%); mp 92.0-94.0° C.
¹H NMR (500 MHz, CDCl₃) δ 7.62 (d, J=8.6, 2H), 7.24 (d, J=8.6, 2H), 3.79 (dd, J=12.6, 6.5 Hz, 1H), 2.64 (dddd, J=14.4, 13.2, 7.0, 0.9 Hz, 1H), 2.42 (ddd, J=14.6, 4.5, 3.0 Hz, 1H), 1.88 (m, 4H), 1.33 (s, 3H), 1.09 (s, 3H). ¹³C{¹H} NMR (126 MHz, CDCl₃) δ 209.36, 144.19, 132.08, 129.74, 118.91, 110.82, 77.27, 77.02, 76.76, 53.19, 47.77, 39.71, 38.35, 31.40, 31.09, 24.23. HRMS (ESI) exact mass calculated for (C₁₅H₁₇NO⁺) requires m/z 228.1383, found m/z 228.1383.

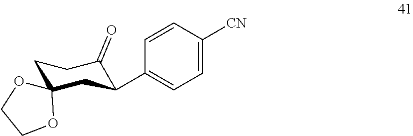

4-(8-oxo-1,4-dioxaspiro[4.5]decan-7-yl)benzonitrile (41): Prepared according to GP-B using Acr 7 (2.8 mg, 0.004 mmol, 0.02 equiv, 2.0 mol %), 4-iodobenzonitrile (45.8 mg, 0.2 mmol, 1.0 equiv), 1,4-cyclohexanedione monoethylene acetal (156.2 mg, 1 mmol, 5.0 equiv), pyrrolidine (84 μL, 1 mmol, 5.0 equiv) and dry MeCN (2 ml, 0.1 M). The reaction was performed at 50° C. for 24 h. After the general workup and purification by flash column chromatography (35% Et₂O/Hexanes) the title compound was isolated as a white solid (32 mg, 62%); mp 142.0-144.0° C.
¹H NMR (500 MHz, CDCl₃) δ 7.63 (d, J=8.6 Hz, 2H), 7.23 (d, J=8.0 Hz, 2H), 4.07 (m, 5H), 2.83 (m, 1H), 2.53 (m, 1H), 2.31 (t, J=13.5 Hz, 1H), 2.24 (m, 1H), 2.15 (m, 2H). ¹³C{¹H} NMR (126 MHz, CDCl₃) δδ 207.54, 142.97, 132.19, 129.63, 118.81, 111.12, 107.00, 64.98, 64.85, 53.24, 41.57, 38.47, 34.71.

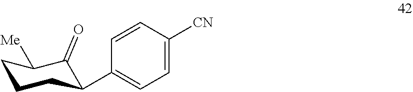

4-(3-methyl-2-oxocyclohexyl)benzonitrile (42): Prepared according to GP-A using Acr 7 (1.4 mg, 0.002 mmol, 0.002 equiv, 0.2 mol %), 4-iodobenzonitrile (229 mg, 1.0 mmol, 1.0 equiv), 2-methylcyclohexanone (0.61 mL, 5 mmol, 5.0 equiv), pyrrolidine (0.42 mL, 5 mmol, 5.0 equiv) and dry MeCN (10 ml, 0.1 M). Workup was performed after 72 h and purification by flash column chromatography (25-30% Et₂O/Hexanes) afforded the title compound as a white solid (68 mg, 32%, >20:1 d.r. determined by crude ¹H NMR in CDCl₃); mp 91.0-93.0° C.
¹H NMR (500 MHz, CDCl₃) δ 7.60 (d, J=8.6 Hz, 2H), 7.23 (d, J=8.1 Hz, 2H), 3.69 (dd, J=12.8, 5.2, 1H), 2.60 (m, 1H), 2.25 (m, 2H), 1.96 (m, 3H), 1.53 (m, 1H), 1.06 (d, J=6.5 Hz, 3H). ¹³C{¹H} NMR (126 MHz, CDCl₃) δ 210.20, 144.36, 131.96, 129.67, 119.01, 110.67, 57.61, 45.90, 37.03, 36.14, 25.56, 14.63. HRMS (ESI) exact mass calculated for (C₁₄H₁₆NO⁺) requires m/z 214.1226, found m/z 214.1227.

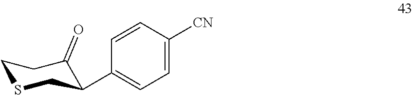

4-(4-oxotetrahydro-2H-thiopyran-3-yl)benzonitrile (43): Prepared according to GP-B using Acr 7 (2.8 mg, 0.004 mmol, 0.02 equiv, 2.0 mol %), 4-iodobenzonitrile (45.8 mg, 0.2 mmol, 1.0 equiv), tetrahydro-4H-thiopyran-4-one (116.2 mg, 1.0 mmol, 5.0 equiv), pyrrolidine (84 μL, 1.0 mmol, 5.0 equiv) and dry MeCN (2 ml, 0.1 M). Workup was performed after 24 h and purification by flash column chromatography (25% Et₂O/Hexanes) afforded the title compound as a light yellow solid (28 mg, 65%); mp 160.0-162.0° C. ¹H NMR (500 MHz, CDCl₃) δ 7.64 (d, J=8.3 Hz, 2H), 7.28 (d, J=8.4 Hz, 2H), 4.03 (dd, J=11.3, 4.6 Hz, 1H), 3.24 (dd, J=13.8, 11.3 Hz, 1H), 3.14 (ddd, J=13.8, 9.3, 6.0 Hz, 1H), 3.06 (m, 2H), 2.87 (m, 2H). ¹³C{¹H} NMR (126 MHz, CDCl₃) δ 206.11, 142.69, 132.31, 129.49, 118.65, 111.48, 59.55, 44.30, 36.55, 30.86. HRMS (ESI) exact mass calculated for (C₁₂H₁₂NOS⁺) requires m/z 218.0634, found m/z 218.0635.
Arylation of 3-methylcyclohexanone:

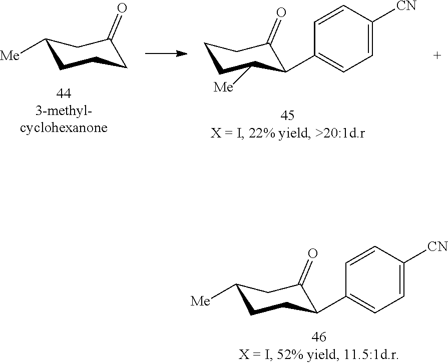

The reaction was performed according to GP-A using Acr 7 (1.4 mg, 0.002 mmol, 0.002 equiv, 0.2 mol %), 4-iodobenzonitrile (229 mg, 1.0 mmol, 1.0 equiv), 3-methylcyclohexanone (0.61 mL, 5.0 mmol, 5.0 equiv), pyrrolidine (0.42 mL, 5.0 mmol, 5.0 equiv) and dry MeCN (10 ml, 0.1 M). After 48 h workup was performed and purification by flash column chromatography (25-30% Et₂O/Hexanes) afforded 4-(4-methyl-2-oxocyclohexyl)benzonitrile (46) as a white solid (111 mg, 52%, 11.5:1 d.r. determined by crude ¹H NMR in CDCl₃); mp 75.0-77.0° C.
4-(4-methyl-2-oxocyclohexyl)benzonitrile (46) : ¹H NMR (500 MHz, CDCl₃) δ 7.62 (d, J=8.5 Hz, 1H), 7.23 (d, J=8.1 Hz, 1H), 3.60 (dd, J=13.6, 5.5 Hz, 1H), 2.54 (ddd, J=13.4, 3.9, 2.3 Hz, 1H), 2.26 (m, 1H), 2.20 (ddd, J=13.5, 12.7, 1.1 Hz, 1H), 2.02 (m, 2H), 1.94 (td, J=13.1, 3.3 Hz, 1H), 1.56 (dtd, J=13.7, 12.4, 3.4 Hz, 1H), 1.10 (d, J=6.3 Hz, 3H). ¹³C{¹H} NMR (126 MHz, CDCl₃) δ 208.84, 144.21, 132.00, 129.50, 118.87, 110.69, 57.37, 42.15, 35.04, 27.64, 25.24.
HRMS (ESI) exact mass calculated for (C₁₄H₁₆NO⁺) requires m/z 214.1226, found m/z 214.1227. The other regioisomer 4-(2-methyl-6-oxocyclohexyl)benzonitrile (45) was also isolated as a mixture with 46. White solid (47 mg, 22%, >20:1 d.r. determined by crude ¹H NMR in CDCl₃).
4-(2-methyl-6-oxocyclohexyl)benzonitrile (45) : ¹H NMR (500 MHz, CDCl₃) δ 7.62 (d, J=8.5 Hz, 2H), 7.18 (d, J=8.2 Hz, 2H), 3.27 (d, J=11.3 Hz, 1H), 2.54 (dddd, J=13.8, 4.5, 2.7, 1.7 Hz, 1H), 2.45 (tdd, J=13.8, 6.1, 1.1 Hz, 1H), 2.04-2.20 (m, 3H), 1.83 (m, 1H), 1.61 (tdd, J=13.4, 11.5, 3.6 Hz, 1H), 0.81 (d, J=6.4 Hz, 3H). ¹³C{¹H} NMR (126 MHz, CDCl₃) δ 208.68, 143.16, 132.03, 130.26, 118.95, 110.81, 65.19, 41.69, 40.80, 34.22, 25.96, 21.10.

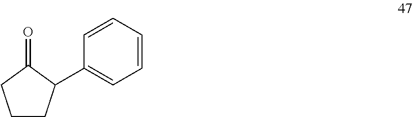 [SMILES: O=C1CCCC1c1ccccc1]

2-phenylcyclopentan-1-one (47): Prepared according to GP-A using Acr 7 (1.4 mg, 0.002 mmol, 0.002 equiv, 0.2 mol %), iodobenzene (204 mg, 1.0 mmol, 1.0 equiv), cyclopentanone (0.44 mL, 5.0 mmol, 5.0 equiv), pyrrolidine (0.42 mL, 5.0 mmol, 5.0 equiv) and dry MeCN (10 ml, 0.1 M). Workup was performed after 24 h and purification by flash column chromatography (10-15% Et₂O/Hexanes) afforded the title compound as a colorless oil (99 mg, 62%).
¹H NMR (500 MHz, CDCl₃) δ 7.34 (t, J=7.5 Hz, 2H), 7.26 (m, 1H), 7.20 (d, J=1.3 Hz, 2H), 3.33 (dd, J=11.1, 8.1 Hz, 1H), 2.50 (m, 2H), 2.30 (m, 1H), 2.14 (m, 2H), 1.95 (m, 1H). ¹³C{¹H} NMR (126 MHz, CDCl₃) δ 218.03, 138.44, 128.60, 128.14, 126.89, 55.31, 38.45, 31.75, 20.86.

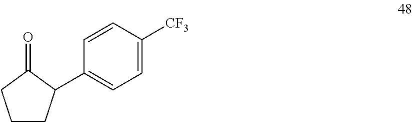 [SMILES: O=C1CCCC1c1ccc(C(F)(F)F)cc1]

2-(4-(trifluoromethyl)phenyl)cyclopentan-1-one (48): Prepared according to GP-A using Acr 7 (1.4 mg, 0.002 mmol, 0.002 equiv, 0.2 mol %), 4-iodobenzotrifluoride (272 mg, 1.0 mmol, 1.0 equiv), cyclopentanone (0.44 mL, 5.0 mmol, 5.0 equiv), pyrrolidine (0.42 mL, 5.0 mmol, 5.0 equiv) and dry MeCN (10 ml, 0.1 M). Workup was performed after 24 h and purification by flash column chromatography (10-15% Et₂O/Hexanes) afforded the title compound as a white solid (142 mg, 63%).
¹H NMR (500 MHz, CDCl₃) δ 7.59 (d, J=8.0 Hz, 2H), 7.32 (dd, J=8.0 Hz, 2H), 3.39 (dd, J=11.7, 8.3 Hz, 1H), 2.53 (m, 2H), 2.31 (m, 1H), 2.20 (m, 1H), 2.12 (m, 1H), 1.97 (m, 1H). ¹³C{¹H} NMR (126 MHz, CDCl₃) δ 216.84, 142.29, 129.2 (q, J=32.5 Hz), 128.51, 125.48 (q, J=3.8 Hz), 124.18 (q, J=272.4 Hz), 55.05, 38.29, 31.43, 20.80. ¹⁹F{¹H} NMR (470 MHz, CDCl₃) δ-62.55.

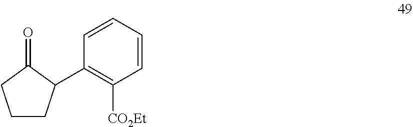 [SMILES: CCOC(=O)c1ccccc1C1CCCC1=O]

Ethyl 2-(2-oxocyclopentyl)benzoate (49): Prepared according to GP-B using Acr 7 (7.0 mg, 0.01 mmol, 0.02 equiv, 2.0 mol %), ethyl 2-iodobenzoate (138 mg, 0.5 mmol, 1.0 equiv), cyclohexanone (260 μL, 2.5 mmol, 5.0 equiv), pyrrolidine (210 μL, 2.5 mmol, 5.0 equiv) and dry MeCN (5 ml, 0.1 M). Workup was performed after 72 h and purification by flash column chromatography (10-20% Et₂O/Hexanes) afforded the title compound as a white solid (50 mg, 43%).
¹H NMR (500 MHz, CDCl₃) δ 7.96 (dd, J=7.9, 1.4 Hz, 1H), 7.46 (td, J=7.6, 1.5 Hz, 1H), 7.31 (td, J=7.7, 1.2 Hz, 1H), 7.17 (dd, J=7.7, 0.9 Hz, 1H), 4.32 (qd, J=7.1, 0.7 Hz, 2H), 4.11 (dd, J=11.6, 8.6 Hz, 1H), 2.46 (m, 3H), 2.18 (m, 2H), 1.96 (m, 1H), 1.37 (t, J=7.1 Hz, 3H). ¹³C{¹H} NMR (126 MHz, CDCl₃) δ 217.40, 167.51, 140.08, 132.14, 130.90, 130.31, 130.00, 126.83, 60.93, 54.84, 38.17, 32.01, 20.98, 14.24.

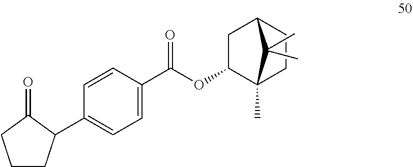 [SMILES: CC1(C)[C@H]2CC[C@]1(C)[C@H](OC(=O)c1ccc(C3CCCC3=O)cc1)C2]

(1S,2R,4S)-1,7,7-trimethylbicyclo[2.2.1]heptan-2-yl 4-(2-oxocyclopentyl)benzoate (50): Prepared according to GP-A using Acr 7 (0.28 mg, 0.0004 mmol, 0.002 equiv, 0.2 mol %), (1S,2R,4S)-1,7,7-trimethylbicyclo[2.2.1]heptan-2-yl 4-ethylbenzoate (76.9 mg, 0.2 mmol, 1.0 equiv), cyclopentanone (45 μL, 1.0 mmol, 5.0 equiv), pyrrolidine (84 μL, 1.0 mmol, 5.0 equiv) and dry MeCN (2 ml, 0.1 M). Workup was performed after 24 h and purification by flash column chromatography (20% Et₂O/Hexanes) afforded the title compound as a mixture of diastereomers (d.r. not determined). White solid (44 mg, 65% yield).
¹H NMR (500 MHz, CDCl₃) δ 8.03 (d, J=8.4 Hz, 2H), 7.28 (d, J=8.0 Hz, 2H), 5.10 (dddd,J=10.0, 3.4, 2.2, 0.9 Hz, 1H), 3.40 (dd, J=11.2, 8.6 Hz, 1H), 2.49 (m, 3H), 2.31 (m, 1H), 2.16 (m, 3H), 1.97 (m, 1H), 1.79 (m, 1H), 1.73 (t, J=4.5 Hz, 1H), 1.40 (m, 1H), 1.29 (ddd, J=12.2, 9.4, 4.5 Hz, 1H), 1.10 (ddd, J=13.9, 3.5, 1.2 Hz, 1H), 0.96 (s, 3H), 0.91 (s, 3H), 0.90 (s, 3H). ¹³C{¹H} NMR (126 MHz, CDCl₃) δ 216.99, 166.62, 143.40, 129.81, 129.57, 128.15, 80.44, 55.32, 49.11, 47.89, 45.00, 38.34, 36.93, 28.10, 27.39, 20.85, 19.73, 18.93, 13.60. HRMS (ESI) exact mass calculated for (C₂₂H₂₉O₃⁺) requires m/z 341.2111, found m/z 341.2111.

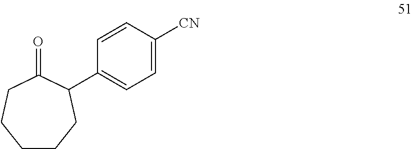 [SMILES: N#Cc1ccc(C2CCCCCC2=O)cc1]

3-(2-oxocycloheptyl)benzonitrile (51): Prepared according to GP-B using Acr 7 (14.0 mg, 0.02 mmol, 0.02 equiv, 2.0 mol %), 4-iodobenzonitrile (229 mg, 1.0 mmol, 1.0 equiv), cycloheptanone (0.59 mL, 5.0 mmol, 5.0 equiv), pyrrolidine (0.42 mL, 5.0 mmol, 5.0 equiv) and dry MeCN (10 ml, 0.1 M). The reaction mixture was heated to 50° C. while irradiating with 518 nm LED. Workup was performed after 96 h and purification by flash column chromatography (30% Et₂O/Hexanes) afforded the title compound as a white solid (73 mg, 34%).
¹H NMR (500 MHz, CDCl₃) δ 7.60 (m, 2H), 7.31 (m, 2H), 3.82 (dd, J=11.2, 3.4 Hz, 1H), 2.62 (m, 2H), 2.00 (m, 5H), 1.70 (m, 1H), 1.50 (m, 2H). ¹³C{¹H} NMR (126 MHz, CDCl₃) δ 211.85, 145.91, 132.15, 128.94, 118.83, 110.72, 58.28, 43.26, 32.22, 29.50, 28.96, 24.54.

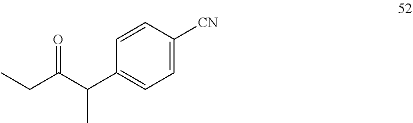

4-(3-oxopentan-2-yl)benzonitrile (52): Prepared according to GP-B using Acr 7 (14.0 mg, 0.02 mmol, 0.02 equiv, 2.0 mol %), 4-iodobenzonitrile (229 mg, 1.0 mmol, 1.0 equiv), 3-pentanone (0.53 mL, 5.0 mmol, 5.0 equiv), pyrrolidine (0.42 mL, 5.0 mmol, 5.0 equiv) and dry MeCN (10 ml, 0.1 M). The reaction mixture was heated to 50° C. while irradiating with 518 nm LED. Workup was performed after 96 h and purification by flash column chromatography (30% Et₂O/Hexanes) afforded the title compound as colorless liquid (58 mg, 31%).
¹H NMR (500 MHz, CDCl₃) δ 7.62 (d, J=8.3 Hz, 2H), 7.34 (d, J=8.3 Hz, 2H), 3.84 (q, J=7.0 Hz, 1H), 2.40 (qd, J=7.3, 3.4 Hz, 2H), 1.41 (d, J=7.0 Hz, 3H), 0.99 (t, J=7.3 Hz, 3H). ¹³C{¹H} NMR (126 MHz, CDCl₃) δ 210.01, 146.07, 132.64, 128.66, 118.62, 111.13, 52.55, 34.79, 17.60, 7.81.

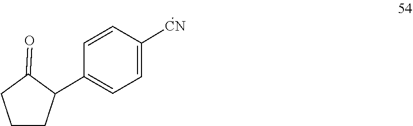

4-(2-oxocyclopentyl)benzonitrile (54): Prepared according to GP-A using Acr 7 (1.4 mg, 0.002 mmol, 0.002 equiv, 0.2 mol %), 4-iodobenzonitrile (229 mg, 1.0 mmol, 1.0 equiv), cyclopentanone (0.44 mL, 5.0 mmol, 5.0 equiv), pyrrolidine (0.42 mL, 5.0 mmol, 5.0 equiv) and dry MeCN (10 ml, 0.1 M). Workup was performed after 24 h and purification by flash column chromatography (25-30% Et₂O/Hexanes) afforded the title compound as a white solid (133 mg, 72%).
¹H NMR (500 MHz, CDCl₃) δ 7.62 (d, J=8.4 Hz, 2H), 7.32 (dd, J=8.6, 0.6 Hz, 2H), 3.39 (dd, J=11.9, 8.4 Hz, 1H), 2.53 (m, 2H), 2.31 (m, 1H), 2.21 (dddt, J=12.8, 8.6, 6.4, 2.0 Hz, 1H), 2.12 (m, 1H), 1.98 (m, 1H). ¹³C{¹H} NMR (126 MHz, CDCl₃) δ 216.33, 143.65, 132.34, 128.99, 118.83, 110.85, 55.07, 38.12, 31.00, 20.61. HRMS (ESI) exact mass calculated for (C₁₂H₁₂NO⁺) requires m/z 186.0913, found m/z 186.0915.

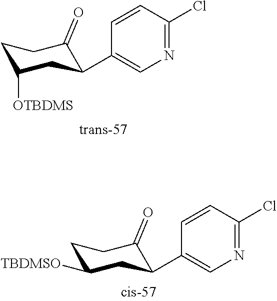

4-((tert-butyldimethylsilyl)oxy)-2-(6-chloropyridin-3-yl)cyclohexan-1-one (57): Prepared according to GP-B using Acr 7 (2.8 mg, 0.004 mmol, 0.002 equiv, 0.2 mol %), 2-chloro-5-iodopyridine (47.9 mg, 0.2 mmol, 1.0 equiv), 4-(tert-butyldimethylsilyloxy)cyclohexanone (228.41, 1.0 mmol, 5.0 equiv), pyrrolidine (84 4, 1.0 mmol, 5.0 equiv) and dry MeCN (2 ml, 0.1 M). Workup was performed after 48 h and purification by flash column chromatography (35-40% Et₂O/Hexanes) afforded the title compound as a white solid (51 mg, 75%).
trans-57: ¹H NMR (500 MHz, CDCl₃) δ 8.13 (d, J=2.5 Hz, 1H), 7.45 (dd, J=8.2, 2.5 Hz, 1H), 7.30 (d, J=8.2 Hz, 1H), 4.30 (p, J=2.8 Hz, 1H), 4.18 (dd, J=13.3, 5.4 Hz, 1H), 2.95 (td, J=13.9, 6.2 Hz, 1H), 2.38 (ddd, J=13.8, 4.7, 2.4 Hz, 1H), 2.23 (ddt, J=13.3, 5.4, 3.4 Hz, 1H), 2.16 (m, 1H), 2.09 (td, J=13.3, 2.1 Hz, 1H), 1.98 (tdd, J=13.9, 4.7, 2.3 Hz, 1H), 0.96 (s, 9H), 0.13 (s, 3H), 0.12 (s, 3H). ¹³C{¹H} NMR (126 MHz, CDCl₃) δ 208.72, 150.07, 149.82, 139.38, 132.91, 123.88, 65.39, 48.08, 42.56, 36.79, 34.83, 25.78, 18.09, -4.86, -4.91.
cis-57: ¹H NMR (500 MHz, CDCl₃) δ 8.13 (d, J=2.4 Hz, 1H), 7.45 (dd, J=8.3, 2.6 Hz, 1H), 7.31 (dd, J=8.2, 0.8 Hz, 1H), 4.26 (tt, J=10.2, 4.2 Hz, 1H), 3.68 (dd, J=13.7, 5.5 Hz, 1H), 2.54 (m, 2H), 2.33 (dddd, J=12.8, 5.5, 4.2, 3.1 Hz, 1H), 2.25 (dddd, J=12.7, 5.2, 4.3, 3.1 Hz, 1H), 2.02 (m, 1H), 1.90 (dtd, J=13.2, 10.3, 8.5 Hz, 1H), 0.89 (s, 9H), 0.11 (s, 3H), 0.10 (s, 3H). ¹³C{¹H} NMR (126 MHz, CDCl₃) δ 207.45, 150.40, 149.82, 139.29, 132.61, 124.14, 69.05, 50.78, 43.01, 38.77, 35.58, 25.89, 18.22, -4.51, -4.55.

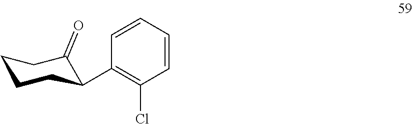

2-(2-chlorophenyl)cyclohexan-1-one (59): Prepared according to GP-A using Acr 7 (7.0 mg, 0.01 mmol, 0.002 equiv, 0.2 mol %), 2-iodochlorobenzene (1.19 g, 5.0 mmol, 1.0 equiv), cyclohexanone (0.52 mL, 25.0 mmol, 5.0 equiv), pyrrolidine (0.42 mL, 25.0 mmol, 5.0 equiv) and dry MeCN (50 ml, 0.1 M). Workup was performed after 48 h and purification by flash column chromatography (5-10% Et₂O/Hexanes) afforded the title compound as a keto-enol mixture (530 mg, 51%).
¹H NMR (500 MHz, CDCl₃) δ 7.37 (m, 1H), 7.26 (m, 1H), 7.21 (m, 3H), 4.11 (dd, J=12.9, 5.3 Hz, 1H), 2.54 (m, 2H), 2.28 (m, 1H), 2.21 (m, 1H), 2.03 (m, 2H), 1.84 (m, 2H). ¹³C{¹H} NMR (126 MHz, CDCl₃) δ 208.78, 136.76, 134.20, 129.43, 129.39, 128.13, 126.77, 54.03, 42.39, 33.94, 27.68, 25.69.

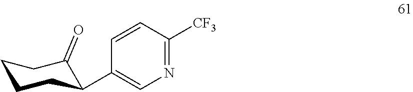

2-(4-(trifluoromethyl)phenyl)cyclohexan-1-one (61): Prepared according to GP-A using Acr 7 (1.4 mg, 0.002 mmol, 0.002 equiv, 0.2 mol %), 4-iodobenzotrifluoride (272 mg, 1.0 mmol, 1.0 equiv), cyclohexanone (0.52 mL, 5.0 mmol, 5.0 equiv), pyrrolidine (0.42 mL, 5.0 mmol, 5.0 equiv) and dry MeCN (10 ml, 0.1 M). Workup was performed after 24 h and purification by flash column chromatography (20% Et₂O/Hexanes) afforded the title compound as a white solid (164 mg, 68%); mp 98.0-100.0° C.
¹H NMR (500 MHz, CDCl₃) δ 7.59 (d, J=8.1 Hz, 2H), 7.26 (d, J=8.1 Hz, 2H), 3.68 (dd, J=12.4, 5.5 Hz, 1H), 2.51 (m, 2H), 2.28 (m, 1H), 2.19 (m, 1H), 2.02 (m, 2H), 1.84 (m, 2H). ¹³C{¹H} NMR (126 MHz, CDCl₃) δ 209.36, 142.80, 129.14(q, J=32.4 Hz), 129.02, 125.26 (q, J=3.8 Hz), 124.25(q, J=271.9 Hz), 57.28, 42.23, 35.24, 27.75, 25.37. ¹⁹F{¹H} NMR (470 MHz, CDCl₃) δ-62.5.

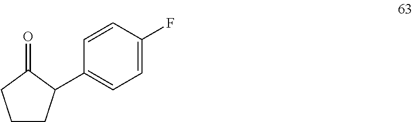 [SMILES: O=C1CCCC1c1ccc(F)cc1]

2-(4-fluorophenyl)cyclopentan-1-one (63): Prepared according to GP-A using Acr 7 (1.4 mg, 0.002 mmol, 0.002 equiv, 0.2 mol %), 4-fluoroiodobenzene (222 mg, 1.0 mmol, 1.0 equiv), cyclopentanone (0.44 mL, 5.0 mmol, 5.0 equiv), pyrrolidine (0.42 mL, 5.0 mmol, 5.0 equiv) and dry MeCN (10 ml, 0.1 M). Workup was performed after 24 h and purification by flash column chromatography (10-15% Et₂O/Hexanes) afforded the title compound as a white solid (119 mg, 67%). NMR (500 MHz, CDCl₃) δ 7.16 (m, 2H), 7.02 (m, 2H), 3.30 (dd, J=12.0, 8.7 Hz, 1H), 2.49 (m, 2H), 2.29 (m, 1H), 2.17 (dddd, J=17.2, 8.6, 4.3, 2.1 Hz, 1H), 2.07 (m, 1H), 1.94 (m, 1H). ¹³C{¹H} NMR (126 MHz, CDCl₃) δ 217.71, 162.82, 160.87, 133.97, 133.95, 129.62, 129.56, 115.51, 115.34, 54.53, 38.24, 31.71, 20.74. ¹⁹F{¹H} NMR (470 MHz, CDCl₃) δ-116.06, -116.07, -116.08, -116.09, -116.10, -116.11, -116.12.

### Procedure for Multi-gram Scale Reaction

An oven-dried 1.0 L schlenk flask equipped with a magnetic stir bar was charged with 70 mg of Acr 7 (0.1 mmol, 0.002 equiv, 0.2 mol %), 11.45 g of 4-iodobenzonitrile (50.0 mmol, 1.0 equiv), 25.9 mL of cyclohexanone (250.0 mmol, 5.0 equiv), 20.9 mL of pyrrolidine (250.0 mmol, 5.0 equiv) and 500 mL of dry MeCN. Then the Schlenk flask was placed in a water bath. Four Kessil 518 nm Green LED lamp was setup approximately 5 cm away from the flask. After 24 h at 20° C., the reaction mixture was evaporated under vacuum. 100 ml 2.0 N HCl was added to the crude reaction mixture followed by addition of 100 ml Et₂O. Aqueous layer was extracted by 3 portions of 100 ml Et₂O. Combined organic layer was washed with brine, dried using Na₂SO_{4,} concentrated under vacuum and purification by flash chromatography (silica gel; 30% Et₂O in hexanes) afforded 4-(2-oxocyclohexyl)benzonitrile (11) as a white crystalline solid (6.95 g, 35 mmol, 70%).

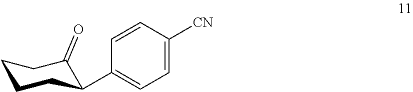

¹H NMR (500 MHz, CDCl₃) δ 7.61 (d, J=8.6 Hz, 2H), 7.24 (d, J=8.1 Hz, 2H), 3.67 (dd, J=12.6, 5.4 Hz, 1H), 2.54 (m, 1H), 2.48 (m, 1H), 2.28 (m, 1H), 2.20 (m, 1h), 2.00 (m, 2H), 1.84 (m, 2H). ¹³C NMR (126 MHz, CDCl₃) δ 208.89, 144.24, 132.09, 129.55, 118.93, 110.81, 57.46, 42.23, 35.12, 27.71, 25.33. HRMS exact mass calculated for (C₁₃H₁₄NO⁺) requires m/z 200.1070, found m/z 200.1069.

### Preparation of Starting Materials

The compounds Si (Yudasaka et al., Org. Lett., 21, 1098-1102, 2019), S2 (Chen et al., J. Am. Chem. Soc., 137, 3338-3351, 2015) and the aryl iodides S3, S4, and S5 (Han et al., Angew. Chem. Int. Ed., 59, 20455-20458, 2020) were prepared according to the previously reported literature.

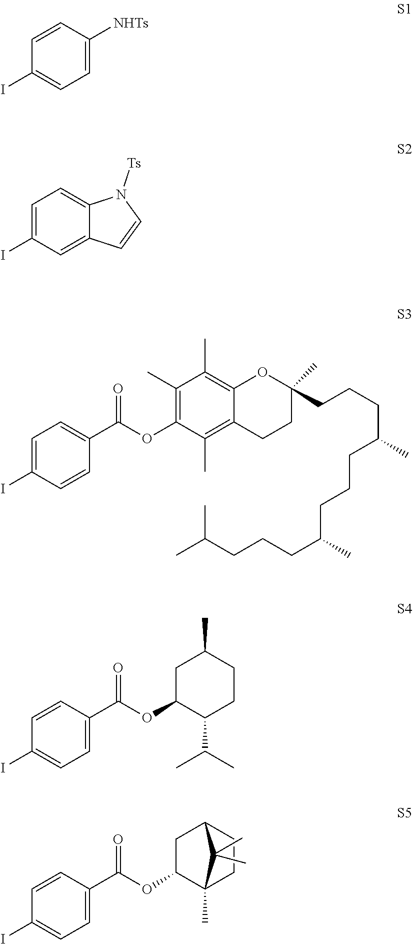

### X-ray Crystallographic Data

Single-crystal X-ray diffraction data of Acr 7 was collected on a Bruker AXS single-crystal system equipped with an Excillum METALJET liquid gallium X-ray source, kappa goniometer, Oxford 800 series cryostream set from 273K to 100 K, and Photon III detector. Image collection, data reduction, and scaling were performed with Bruker AXS APEX3 software. The resolution of the solid-state structure was accomplished using the SHELXS-97 and SHELXT program. The refinement was performed with the SHELXL program and the structure solution and the refinement were achieved with the PLATON and Olex2 software. All atoms—except hydrogens—were refined anisotropically. The positions of the hydrogen atoms were determined using residual electronic densities, which are calculated by a Fourier difference. A final weighting step was performed, followed by multiples loops of refinement.

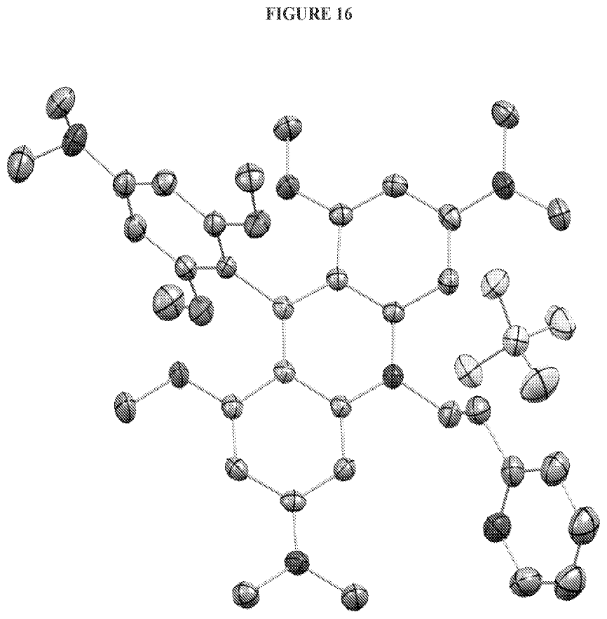

Dark orange crystals of Acr 7 were grown by slow evaporation using DCM and hexanes at ambient temperature. See FIG. 16.

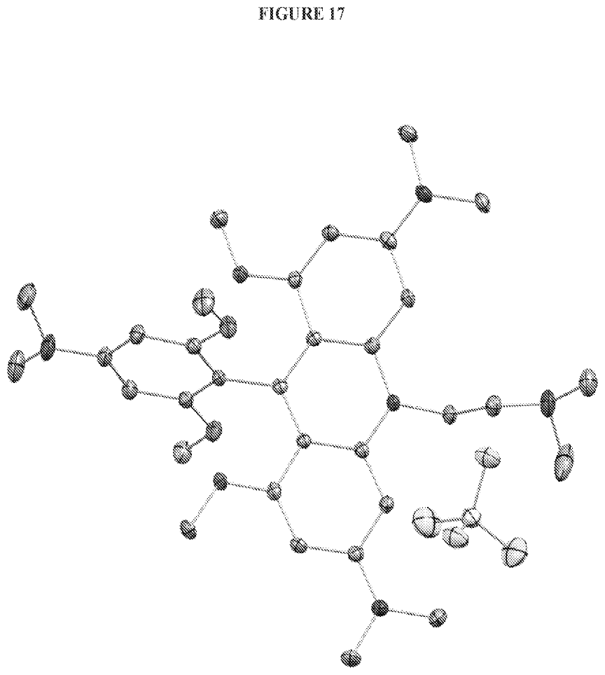

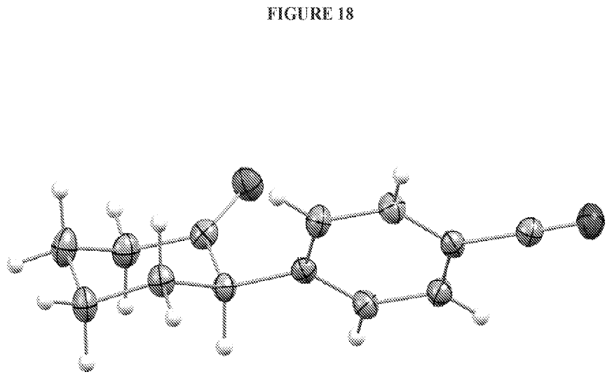

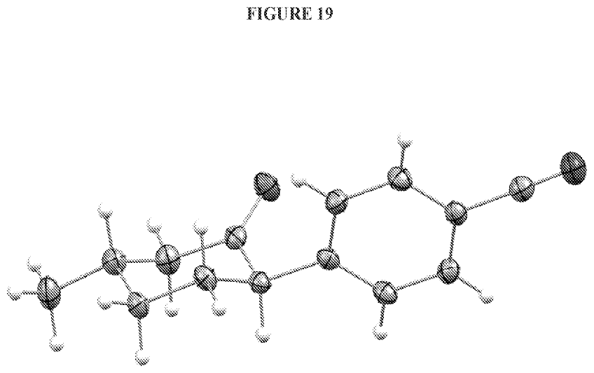

Single-crystal X-ray diffraction data of Acr 8, and compounds 11 and 49 were collected on a Bruker Kappa APEX II CCD diffractometer using Mo Kα radiation (λ=0.71073 Å) radiation, kappa goniometer, Oxford 800 series cryostream set from 273 K to 100 K, and Photon III detector. See FIGS. 17, 18 and 19, respectively.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

1. A method for producing an α-aryl substituted carbonyl compound, the method comprises irradiating a solution mixture comprising:
an unactivated carbonyl compound;
an optionally substituted aryl halide or an optionally substituted heteroaryl halide;
an amine compound; and
a photocatalyst;
with visible light under conditions sufficient to produce the α-aryl substituted carbonyl compound.

2. The method of claim 1, wherein the optionally substituted aryl halide or optionally substituted heteroaryl halide is of the formula: 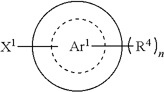
wherein n is 0 to x, where x is the maximum number of substituents possible on Ar¹;
X¹ is Cl, Br or I;
Ar¹ is aryl or heteroaryl; and
each R⁴ is, independently, alkyl, halide, haloalkyl, a carbonyl group, cyano, —CO₂R^{a}, —OR^{b}, —NR^{c}R^{d}, an amide, —SR^{a}, —BR^{c}R f , or —PR^{c}R^{f}, wherein each R^{a} is independently, alkyl, R^{b} is alkyl or a hydroxy protecting group, R^{c} is H or alkyl, and R^{d} is H, alkyl or an amine protecting group, R^{c} and R^{f} are each independently H, alkyl, hydroxy or an alkoxy group, or R^{e} and R^{f} together with the atom to which they are attached form an optionally substituted cyclic moiety.

3. A method for producing an α-aryl substituted compound of formula (I): 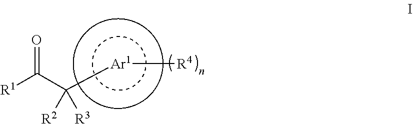
the method comprising irradiating an electromagnetic radiation to a solution mixture comprising:
a carbonyl compound of the formula: 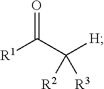
an aryl halide or heteroaryl halide compound of the formula: 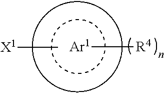
an amine compound;
and a photocatalyst,
under conditions sufficient to produce said α-aryl substituted compound of Formula I, wherein R¹ is H, alkyl, alkoxyl or aryl;
each of R² and R³ is independently H, alkyl, or aryl; or R¹ and R² together with the atoms to which they are attached to form an optionally substituted cycloalkyl moiety;
Ar';
An is aryl or heteroaryl, each of which is optionally substituted;
n is an integer from 0 to x, where x is a maximum number of substituents possible on X¹ is Cl, Br, or I; and
each R⁴ is independently alkyl, halide, haloalkyl, carbonyl group, cyano, —CO₂R^{a}, —OR^{b}, —NR^{c}R^{d}, an amide, —SR^{a}, —BR^{e}R^{f}, —PR^{e}R^{f} where R^{a} is alkyl, R^{b} is alkyl or a hydroxy protecting group, R^{c} is H or alkyl, and R^{d} is H, alkyl, or an amine protecting group, R^{e} and R^{f} are each independently H, alkyl, hydroxy or an alkoxy group, or R^{e} and R^{f} together with the atom to which they are attached to form an optionally substituted cyclic moiety.

4. The method of claim 3, wherein Ar¹ is phenyl, naphthyl, anthracyl, pyridyl, furanyl, thiophenyl, thiazolyl, isothiazolyl, triazolyl, triazinyl, imidazolyl, oxazolyl, isoxazolyl, pyrrolyl, pyrazolyl, pyrazinyl, pyrimidinyl, benzofuranyl, isobenzofuranyl, benzothiazolyl, benzoisothiazolyl, benzotriazolyl, indolyl, isoindolyl, benzoxazolyl, thiazolyl, isothiazolyl, quinolyl, isoquinolyl, benzimidazolyl, benzisoxazolyl, benzothiophenyl, dibenzofuran, or benzodiazepin-2-one-5-yl, each of which may be optionally substituted.

5. A method for producing an isocoumarin compound of the formula: 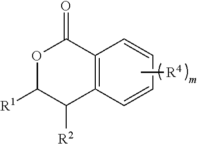
the method comprising irradiating an electromagnetic radiation to a solution mixture comprising:
a carbonyl compound of the formula: 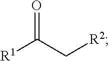
an aryl halide compound of the formula: 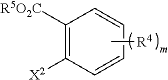
an amine compound; and
a photocatalyst, under conditions sufficient to produce said isocoumarin compound, wherein R¹ is H, alkyl, alkoxyl or aryl;
R² is H, alkyl, or aryl; or R¹ and R² together with the atoms to which they are attached to form an optionally substituted cycloalkyl moiety;
m is an integer from 0 to 4;
X² is Cl, Br, or I;
each R⁴ is independently alkyl, halide, haloalkyl, a carbonyl group, cyano, —CO₂R^{a}, —OR^{b}, —NR^{e}R^{d}, amide, —SR^{a}, —BR^{e}R^{f}, —PR^{e}R^{f} where R^{a} is alkyl, R^{b} is alkyl or a hydroxy protecting group, R^{e} is H or alkyl, and R^{d} is H, alkyl, or an amine protecting group, R^{e} and R^{f} is independently H, alkyl, hydroxy or an alkoxy group or R^{e} and R^{f} together with the atoms to which they are attached to form an optionally substituted cyclic moiety; and
R⁵ is H, alkyl or aryl.

6. A method for producing an α-aryl substituted cyclic ketone, the method comprising irradiating a solution mixture comprising:
a cyclic ketone compound;
an aryl halide;
a secondary amine compound; and
a photocatalyst comprising an acridinium, helicenium, angulenium, or a combination thereof;
with light under conditions sufficient to produce an α-aryl substituted cyclic ketone.

7. The method of claim 6, wherein the cyclic ketone compound is an unactivated cyclic ketone compound.

8. The method of claim 6, wherein the cyclic ketone compound comprises a heterocycloalkyl ketone or a cycloalkyl ketone.

9. The method of claim 6, wherein the cyclic ketone is a substituted cyclic ketone.

10. The method of claim 6, wherein said cyclic ketone is a 5-, 6-, or 7-membered cyclic ketone.

11. The method of claim 6, wherein the α-aryl substituted cyclic ketone is produced in a diastereomeric excess of at least about 50% d.e.

12. The method of claim 6, wherein said heterocycloalkyl ketone comprises a heterocyclic moiety selected from the group consisting of tetrahydro-2H-thiopyran, piperidine, tetrahydro-2H-pyran, tetrahydrothiophene, pyrrolidine, tetrahydrofuran, azepane, thiepane, and oxepane.

13. The method of claim 6, wherein the photocatalyst is selected from the group consisting of acridinium based photocatalysts, a 9-mesityl-acridinium compound; heliceniums; anguleniums; xanthene based photocatalysts, eosin Y; carbazolyl based photocatalysts; quinoliniums; benzophenones;
quinones; pyryliums; cyanoarenes; perylene diimides; Ru-based photocatalysts; Ir based photocatalysts or any combination thereof.

14. The method of claim 6, wherein the photocatalyst comprises an acridinium of formula (I): 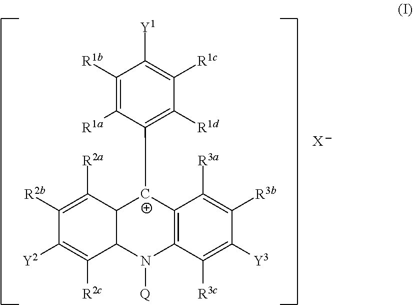
wherein each of R^{1a}, R^{1b}, R^{1c}, R^{1d}, R^{2a}, R^{2b}, R^{2c}, R^{3a}, R^{3b} and R^{3c} is independently H, halide, haloalkyl (e.g., CF3), 'NR a R b , C₁-C₁₂ alkyl, C₁-C₄ alkoxy, —NO₂, —CN, —CO₂R (wherein R is H or C₁-C₄ alkyl), or An, wherein each of R^{a} and R^{b} is independently H or C₁-C₄ alkyl;
each of Y¹, Y 2 and Y 3 is independently —NR^{4a}R^{4b}OR^{4a}, or R^{4a}, wherein each of R^{4a} and R^{4b} is independently C₁-C₁₂ alkyl, haloalkyl, or aryl;
Q is C₁-C₁₂ alkyl, a moiety of the formula —L—Z, wherein L is C₂-C₁₂ alkylene, and Z is amino, monoalkylamino, dialkylamino, or pyridyl; and
X⁻ is a counter anion.

15. The method of claim 6 any one of the preceding claims, wherein the photocatalyst comprises Acr6, Acr 7, Acr8, or any combination thereof.

16. The method of claim 6 any one of the preceding claims, wherein said light electromagnetic radiation is a visible light.

17. The method of claim 6, wherein the aryl halide has a reduction potential less than or equal to excited state oxidation potential of said photocatalyst.

18. The method of claim 6, wherein the wavelength of said light is from about 440 nm to about 650 nm.

19. The method of claim 6, wherein the amount of the photocatalyst used is in the range of from about 0.001 equivalents to about 0.1 equivalents relative to the amount of the aryl halide compound.

20. The method of claim 3, wherein the amount of the amine compound ranges from about 1 equivalent to about 10 equivalents relative to the amount of the aryl halide.

21. The method of claim 3, wherein the amine compound is a 5, 6 or 7-membered cyclic amine compound.

22. The method of claim 3, wherein the amine compound comprises azepane, piperidine, pyrrolidine, or any combination thereof.
